(19) United States
(12) Patent Application Publication
(10) Pub. No.: US 2010/0130537 A1
(43) Pub. Date: May 27, 2010

(54) CINNAMIDE COMPOUNDS FOR DEMENTIA

(75) Inventor: Takehiko Miyagawa, Ibaraki (JP)

Correspondence Address: BIRCH STEWART KOLASCH & BIRCH, PO BOX 747, FALLS CHURCH, VA, 22040-0747, US

(73) Assignee: EISAI R&D MANAGEMENT CO., LTD, TOKYO (JP)

(21) Appl. No.: 12/594,172

(22) PCT Filed: Apr. 25, 2008
(86) PCT No.: PCT/JP2008/058452

§ 371 Date: Sep. 30, 2009

Related U.S. Application Data
(60) Provisional application No. 60/924,009, filed on Apr. 26, 2007.

(51) Int. Cl. A61K 31/435; A61K 31/454; A61P 25/00; A61P 25/28

(52) U.S. Cl. 514/295; 514/297; 514/326

(57) ABSTRACT

The invention provides methods for treating, preventing, and delaying the onset of dementia and mild cognitive impairments by administering to a patient in need thereof at least one cinnamide compound and one or more second-line active ingredients, such as cholinesterase inhibitors; AMPA receptor antagonists; NMDA receptor antagonists; and the like. The invention also provides pharmaceutical compositions, combinations, and kits.

### RELATED APPLICATION

This application claims priority under 35 U.S.C. §119 to U.S. Provisional Application No. 60/924,009 filed Apr. 26, 2007, the disclosures of which are incorporated by reference herein in their entirety.

### FIELD OF THE INVENTION

The invention provides pharmaceutical compositions, combinations, and kits comprising cinnamide compounds, and methods for treating dementia using cinnamide compounds.

### BACKGROUND OF THE INVENTION

Cholinesterase inhibitors have conventionally been used to treat dementia, such as Alzheimer's Disease and Senile Dementia of the Alzheimer's Type. In particular, donepezil hydrochloride that acts as an acetylcholinesterase inhibitor increases acetylcholine in the brain and is used extensively as a drug for treating Alzheimer's senile dementia. Donepezil is described in U.S. Pat. No. 4,895,841. Other drugs including N-methyl-D-aspartic acid (NMDA) receptor antagonists are also used. Nonetheless, novel drugs and treatment methods are needed.

Memantine hydrochloride (NAMENDA®, Forest Pharmaceuticals, Inc.; AXURA®, Merz Pharmaceuticals), which is an NMDA receptor antagonist, is an amantadine derivative and is known to protect nerve cells and improves the symptoms of Parkinson's disease. Recently developed as a drug for treating moderate to severe Alzheimer's disease, memantine hydrochloride is provided in a liquid form or as film-coated tablets.

1,2-dihydropyridine compounds are known as α-amino-3-hydroxy-5-methyl-4-isoxazole propionic acid (AMPA) receptor antagonists and are described in U.S. Pat. No. 6,949,571. Methods for treating diseases and administering these compounds in conjunction with a cholinesterase inhibitor are described in WO 2006/107859.

Cinnamide compounds are known as agents for inhibiting the production of amyloid beta 40 and amyloid beta 42 from amyloid precursor proteins. Cinnamide compounds are effective for treating neurodegenerative diseases caused by amyloid beta, such as Alzheimer's disease and Down's syndrome. Cinnamide compounds are described, for example in US Publication No. 2006/0004013 and PCT Publication No. WO 2006/046575.

There is a need in the art for treating dementia using novel pharmaceutical compositions or combinations. The invention is directed to these, as well as other, important goals.

### SUMMARY OF THE INVENTION

The invention provides methods for treating dementia and/or mild cognitive impairments; methods for the prophylaxis of dementia and/or mild cognitive impairments; and methods for delaying the onset of dementia and/or mild cognitive impairments in a patient in need thereof by administering at least one cinnamide compound and one or more second-line active ingredients selected from the group consisting of cholinesterase inhibitors; AMPA receptor antagonists; NMDA receptor antagonists; pramiracetam; aniracetam; acetylcholine releasing stimulants; calcium channel agonists; free radical scavengers; platelet activating factor antagonists; platelet aggregation antagonists; insulin sensitizers; peroxisome proliferator-activated receptor agonists; peroxisome proliferator-activated receptor gamma agonists; monoamine oxidase B inhibitors; carnitine acetyltransferase stimulants; NSAIDs; nerve growth factor agonists; beta-amyloid inhibitors; immunomodulators; NF-kappa B inhibitors; thyrotropin releasing hormone agonists; dopamine D2 receptor antagonists; serotonin 2 receptor antagonists; muscarinic M1 receptor agonists; alpha 1 adrenoceptor agonists; serotonin 3 receptor antagonists; dopamine D2 receptor agonists; dopamine D2 receptor antagonists; serotonin 1A receptor agonists; serotonin 2A receptor antagonists; glucocorticoid antagonists; progesterone antagonists; HMG-CoA reductase inhibitors; adenosine uptake inhibitors; phosphodiesterase inhibitors; acetylcholine receptor agonists; membrane permeability enhancers; cannabinoid 1 receptor antagonists; cannabinoid receptor agonists; angiogenesis inhibitors; immunosuppressants; tubulin antagonists; thromboxane A2 synthase inhibitors; antioxidants; alpha adrenoreceptor antagonists; estrogen agonists; 3-beta hydroxysteroid dehydrogenase inhibitors; signal transduction pathway inhibitors; melatonin receptor agonists; immunostimulants; HIV entry inhibitors; sodium channel antagonists; microtubule inhibitors; glycine NMDA agonists; adenosine A1 receptor antagonists; ATPase stimulants; mitochondrial function enhancers; and growth hormone releasing factor agonists.

The invention provides pharmaceutical combinations, pharmaceutical compositions, and kits comprising (i) at least one cinnamide compound; and (ii) one or more compounds selected from the group consisting of the second-line active ingredients described herein. The pharmaceutical combinations may comprise two or more formulations comprising active ingredients that may be separately administered (e.g., simultaneously, sequentially) to a patient. The pharmaceutical compositions may comprise two or more active ingredients.

It has been unexpectedly discovered that the combination of the cinnamide compound and one or more of the second-line compounds produces synergistic effects in the treatment and/or prophylaxis of dementia or mild cognitive impairments, and in delaying the onset of dementia or mild cognitive impairments.

The present invention relates to the following.

(1) A pharmaceutical composition comprising:

(A) a compound of Formula (I) or a pharmaceutically acceptable salt thereof:

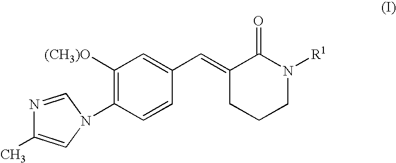

wherein R¹ is:

(a) —X₁—Ar₁, wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

(b) an indenyl group optionally substituted with 1 to 3 halogen atoms;

(c) a tetrahydronaphthyl group optionally substituted with 1 to 3 halogen atoms; or

(d) a chromanyl group optionally substituted with 1 to 3 halogen atoms;

(B) a cholinesterase inhibitor; an AMPA receptor antagonist; an NMDA receptor antagonist; or a mixture or combination of two or more thereof; and
(C) one or more pharmaceutically acceptable carriers.

(2) The pharmaceutical composition of (1), wherein R¹ is —X₁—Ar₁; wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

(3) The pharmaceutical composition of (1), wherein R¹ is an indenyl group optionally substituted with 1 to 3 halogen atoms; a tetrahydronaphthyl group optionally substituted with 1 to 3 halogen atoms; or a chromanyl group optionally substituted with 1 to 3 halogen atoms.

(4) The pharmaceutical composition of (1), wherein (A) is at least one compound selected from: (E)-1-(3,4-difluorobenzyl)-3-[3-methoxy-4-(4-methyl-1H-imidazol-1-yl)benzylidene]piperidin-2-one or a pharmaceutically acceptable salt thereof;
(E)-1-indan-2-yl-3-[3-methoxy-4-(4-methyl-1H-imidazol-1-yl)benzylidene]piperidin-2-one or a pharmaceutically acceptable salt thereof;
(E)-1-[(4R)-chroman-4-yl]-3-[3-methoxy-4-(4-methyl-1H-imidazol-1-yl)benzylidene]piperidin-2-one or a pharmaceutically acceptable salt thereof; (E)-1-[(4S)-chroman-4-yl]-3-[3-methoxy-4-(4-methyl-1H-imidazol-1-yl)benzylidene]piperidin-2-one or a pharmaceutically acceptable salt thereof;
(E)-1-[(1S)-1-(4-fluorophenyl)ethyl]-3-[3-methoxy-4-(4-methyl-1H-imidazol-1-yl)benzylidene]piperidin-2-one or a pharmaceutically acceptable salt thereof;
(E)-1-(4-tert-butylbenzyl)-3-[3-methoxy-4-(4-methyl-1H-imidazol-1-yl)benzylidene]piperidin-2-one or a pharmaceutically acceptable salt thereof;
(E)-1-[(2S)-5-fluoroindan-2-yl]-3-[3-methoxy-4-(4-methyl-1H-imidazol-1-yl)benzylidene]piperidin-2-one or a pharmaceutically acceptable salt thereof; (E)-1-[(2R)-5-fluoroindan-2-yl]-3-[3-methoxy-4-(4-methyl-1H-imidazol-1-yl)benzylidene]piperidin-2-one or a pharmaceutically acceptable salt thereof;
(E)-1-[(4R)-7-fluorochroman-4-yl]-3-[3-methoxy-4-(4-methyl-1H-imidazol-1-yl)benzylidene]piperidin-2-one or a pharmaceutically acceptable salt thereof; or (E)-1-[(4S)-7-fluorochroman-4-yl]-3-[3-methoxy-4-(4-methyl-1H-imidazol-1-yl)benzylidene]piperidin-2-one or a pharmaceutically acceptable salt thereof;
(E)-3-[3-methoxy-4-(4-methyl-1H-imidazol-1-yl)benzylidene]-1-[(2R)-1,2,3,4-tetrahydronaphthalen-2-yl]piperidin-2-one or a pharmaceutically acceptable salt thereof; (E)-3-[3-methoxy-4-(4-methyl-1H-imidazol-1-yl)benzylidene]-1-[(2S)-1,2,3,4-tetrahydronaphthalen-2-yl]piperidin-2-one or a pharmaceutically acceptable salt thereof; and
(E)-1-[(1R)-1-(2,4-difluorophenyl)ethyl]-3-[3-methoxy-4-(4-methyl-1H-imidazol-1-yl)benzylidene]piperidin-2-one or a pharmaceutically acceptable salt thereof; or (E)-1-[(1S)-1-(2,4-difluorophenyl)ethyl]-3-[3-methoxy-4-(4-methyl-1H-imidazol-1-yl)benzylidene]piperidin-2-one or a pharmaceutically acceptable salt thereof.

(5) The pharmaceutical composition of (1), wherein (B) is one or more compounds selected from the group consisting of donepezil or a pharmaceutically acceptable salt thereof, huperzine A or a pharmaceutically acceptable salt thereof, tacrine or a pharmaceutically acceptable salt thereof, rivastigmine or a pharmaceutically acceptable salt thereof, galantamine or a pharmaceutically acceptable salt thereof, pramiracetam or a pharmaceutically acceptable salt thereof, aniracetam or a pharmaceutically acceptable salt thereof, nefiracetam or a pharmaceutically acceptable salt thereof, EGb 761 or a pharmaceutically acceptable salt thereof, rosiglitazone or a pharmaceutically acceptable salt thereof, rasagiline or a pharmaceutically acceptable salt thereof, levacecarnine or a pharmaceutically acceptable salt thereof, celecoxib or a pharmaceutically acceptable salt thereof, 3-(2-cyanophenyl)-5-(2-pyridyl)-1-phenyl-1,2-dihydropyridin-2-one, a pharmaceutically acceptable salt thereof or a hydrate thereof, talampanel or a pharmaceutically acceptable salt thereof, becampanel or a pharmaceutically acceptable salt thereof, memantine or a pharmaceutically acceptable salt thereof, neramexane or a pharmaceutically acceptable salt thereof, xaliproden or a pharmaceutically acceptable salt thereof, tarenflurbil or a pharmaceutically acceptable salt thereof, tramiprosate or a pharmaceutically acceptable salt thereof, and leuprorelin-D or a pharmaceutically acceptable salt thereof.

(6) The pharmaceutical composition of (1), wherein (B) is one or more compounds selected from the group consisting of donepezil or a pharmaceutically acceptable salt thereof, 3-(2-cyanophenyl)-5-(2-pyridyl)-1-phenyl-1,2-dihydropyridin-2-one, a pharmaceutically acceptable salt thereof or a hydrate thereof and memantine or a pharmaceutically acceptable salt thereof.

(7) The pharmaceutical composition of (1), wherein (B) is donepezil or a pharmaceutically acceptable salt thereof.

(8) The pharmaceutical composition of (1), wherein the composition is used for: treating dementia or one or more mild cognitive impairments; the prophylaxis of dementia or one or more mild cognitive impairments; or delaying the onset of dementia or one or more mild cognitive impairments.

(9) A combination comprising:

(A) a compound of Formula (I) or a pharmaceutically acceptable salt thereof:

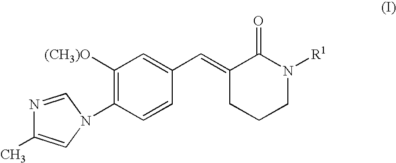

wherein R¹ is:

(a) —X₁—Ar₁, wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

(b) an indenyl group optionally substituted with 1 to 3 halogen atoms;

(c) a tetrahydronaphthyl group optionally substituted with 1 to 3 halogen atoms; or

(d) a chromanyl group optionally substituted with 1 to 3 halogen atoms; and

(B) a cholinesterase inhibitor; an AMPA receptor antagonist; an NMDA receptor antagonist; or a mixture or combination of two or more thereof.

(10) The combination of (9), wherein R¹ is —X₁—Ar₁; wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

(11) The combination of (9), wherein R¹ is an indenyl group optionally substituted with 1 to 3 halogen atoms; a tetrahydronaphthyl group optionally substituted with 1 to 3 halogen atoms; or a chromanyl group optionally substituted with 1 to 3 halogen atoms.

(12) The combination of (9), wherein (A) is at least one compound selected from:
(E)-1-(3,4-difluorobenzyl)-3-[3-methoxy-4-(4-methyl-1H-imidazol-1-yl)benzylidene]piperidin-2-one or a pharmaceutically acceptable salt thereof;
(E)-1-indan-2-yl-3-[3-methoxy-4-(4-methyl-1H-imidazol-1-yl)benzylidene]piperidin-2-one or a pharmaceutically acceptable salt thereof;
(E)-1-[(4R)-chroman-4-yl]-3-[3-methoxy-4-(4-methyl-1H-imidazol-1-yl)benzylidene]piperidin-2-one or a pharmaceutically acceptable salt thereof; (E)-1-[(4S)-chroman-4-yl]-3-[3-methoxy-4-(4-methyl-1H-imidazol-1-yl)benzylidene]piperidin-2-one or a pharmaceutically acceptable salt thereof;
(E)-1-[(1S)-1-(4-fluorophenyl)ethyl]-3-[3-methoxy-4-(4-methyl-1H-imidazol-1-yl)benzylidene]piperidin-2-one or a pharmaceutically acceptable salt thereof;
(E)-1-(4-tert-butylbenzyl)-3-[3-methoxy-4-(4-methyl-1H-imidazol-1-yl)benzylidene]piperidin-2-one or a pharmaceutically acceptable salt thereof;
(E)-1-[(2S)-5-fluoroindan-2-yl]-3-[3-methoxy-4-(4-methyl-1H-imidazol-1-yl)benzylidene]piperidin-2-one or a pharmaceutically acceptable salt thereof; (E)-1-[(2R)-5-fluoroindan-2-yl]-3-[3-methoxy-4-(4-methyl-1H-imidazol-1-yl)benzylidene]piperidin-2-one or a pharmaceutically acceptable salt thereof;
(E)-1-[(4R)-7-fluorochroman-4-yl]-3-[3-methoxy-4-(4-methyl-1H-imidazol-1-yl)benzylidene]piperidin-2-one or a pharmaceutically acceptable salt thereof; or (E)-1-[(4S)-7-fluorochroman-4-yl]-3-[3-methoxy-4-(4-methyl-1H-imidazol-1-yl)benzylidene]piperidin-2-one or a pharmaceutically acceptable salt thereof;
(E)-3-[3-methoxy-4-(4-methyl-1H-imidazol-1-yl)benzylidene]-1-[(2R)-1,2,3,4-tetrahydronaphthalen-2-yl]piperidin-2-one or a pharmaceutically acceptable salt thereof; (E)-3-[3-methoxy-4-(4-methyl-1H-imidazol-1-yl)benzylidene]-1-[(2S)-1,2,3,4-tetrahydronaphthalen-2-yl]piperidin-2-one or a pharmaceutically acceptable salt thereof; and
(E)-1-[(1R)-1-(2,4-difluorophenyl)ethyl]-3-[3-methoxy-4-(4-methyl-1H-imidazol-1-yl)benzylidene]piperidin-2-one or a pharmaceutically acceptable salt thereof; or (E)-1-[(1S)-1-(2,4-difluorophenyl)ethyl]-3-[3-methoxy-4-(4-methyl-1H-imidazol-1-yl)benzylidene]piperidin-2-one or a pharmaceutically acceptable salt thereof.

(13) The combination of (9), wherein (B) is one or more compounds selected from the group consisting of donepezil or a pharmaceutically acceptable salt thereof, huperzine A or a pharmaceutically acceptable salt thereof, tacrine or a pharmaceutically acceptable salt thereof, rivastigmine or a pharmaceutically acceptable salt thereof, galantamine or a pharmaceutically acceptable salt thereof, pramiracetam or a pharmaceutically acceptable salt thereof, aniracetam or a pharmaceutically acceptable salt thereof, nefiracetam or a pharmaceutically acceptable salt thereof, EGb 761 or a pharmaceutically acceptable salt thereof, rosiglitazone or a pharmaceutically acceptable salt thereof, rasagiline or a pharmaceutically acceptable salt thereof, levacecarnine or a pharmaceutically acceptable salt thereof, celecoxib or a pharmaceutically acceptable salt thereof, 3-(2-cyanophenyl)-5-(2-pyridyl)-1-phenyl-1,2-dihydropyridin-2-one, a pharmaceutically acceptable salt thereof or a hydrate thereof, talampanel or a pharmaceutically acceptable salt thereof, becampanel or a pharmaceutically acceptable salt thereof, memantine or a pharmaceutically acceptable salt thereof, neramexane or a pharmaceutically acceptable salt thereof, xaliproden or a pharmaceutically acceptable salt thereof, tarenflurbil or a pharmaceutically acceptable salt thereof, tramiprosate or a pharmaceutically acceptable salt thereof, and leuprorelin-D or a pharmaceutically acceptable salt thereof.

(14) The combination of (9), wherein (B) is one or more compounds selected from the group consisting of donepezil or a pharmaceutically acceptable salt thereof, 3-(2-cyanophenyl)-5-(2-pyridyl)-1-phenyl-1,2-dihydropyridin-2-one, a pharmaceutically acceptable salt thereof or a hydrate thereof and memantine or a pharmaceutically acceptable salt thereof.

(15) The combination of (9), wherein (B) is donepezil or a pharmaceutically acceptable salt thereof.

(16) The combination of (9), wherein (A) and (B) are administered separately to a patient or are administered to a patient in the form of a pharmaceutical composition.

(17) The combination of (9), wherein the combination is used for treating dementia or one or more mild cognitive impairments; for the prophylaxis of dementia or one or more mild cognitive impairments; or delaying the onset of dementia or one or more mild cognitive impairments.

(18) Use of compounds (A) and (B) for producing a pharmaceutical composition in the treatment of dementia or one or more mild cognitive impairments; for the prophylaxis of dementia or one or more mild cognitive impairments; or delaying the onset of dementia or one or more mild cognitive impairments, wherein (A) and (B) are:

(A) a compound of Formula (I) or a pharmaceutically acceptable salt thereof:

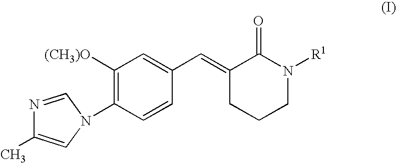

wherein R¹ is:

(a) —X₁—Ar₁, wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

(b) an indenyl group optionally substituted with 1 to 3 halogen atoms;

(c) a tetrahydronaphthyl group optionally substituted with 1 to 3 halogen atoms; or

(d) a chromanyl group optionally substituted with 1 to 3 halogen atoms; and

(B) a cholinesterase inhibitor; an AMPA receptor antagonist; an NMDA receptor antagonist; or a mixture or combination of two or more thereof.

(19) The use of (18), wherein R¹ is —X₁—Ar₁; wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

(20) The use of (18), wherein R¹ is an indenyl group optionally substituted with 1 to 3 halogen atoms; a tetrahydronaphthyl group optionally substituted with 1 to 3 halogen atoms; or a chromanyl group optionally substituted with 1 to 3 halogen atoms.

(21) The use of (18), wherein (A) is at least one compound selected from: (E)-1-(3,4-difluorobenzyl)-3-[3-methoxy-4-(4-methyl-1H-imidazol-1-yl)benzylidene]piperidin-2-one or a pharmaceutically acceptable salt thereof;
(E)-1-indan-2-yl-3-[3-methoxy-4-(4-methyl-1H-imidazol-1-yl)benzylidene]piperidin-2-one or a pharmaceutically acceptable salt thereof;
(E)-1-[(4R)-chroman-4-yl]-3-[3-methoxy-4-(4-methyl-1H-imidazol-1-yl)benzylidene]piperidin-2-one or a pharmaceutically acceptable salt thereof; (E)-1-[(4S)-chroman-4-yl]-3-[3-methoxy-4-(4-methyl-1H-imidazol-1-yl)benzylidene]piperidin-2-one or a pharmaceutically acceptable salt thereof;
(E)-1-[(1S)-1-(4-fluorophenyl)ethyl]-3-[3-methoxy-4-(4-methyl-1H-imidazol-1-yl)benzylidene]piperidin-2-one or a pharmaceutically acceptable salt thereof;
(E)-1-(4-tert-butylbenzyl)-3-[3-methoxy-4-(4-methyl-1H-imidazol-1-yl)benzylidene]piperidin-2-one or a pharmaceutically acceptable salt thereof;
(E)-1-[(2S)-5-fluoroindan-2-yl]-3-[3-methoxy-4-(4-methyl-1H-imidazol-1-yl)benzylidene]piperidin-2-one or a pharmaceutically acceptable salt thereof; (E)-1-[(2R)-5-fluoroindan-2-yl]-3-[3-methoxy-4-(4-methyl-1H-imidazol-1-yl)benzylidene]piperidin-2-one or a pharmaceutically acceptable salt thereof;
(E)-1-[(4R)-7-fluorochroman-4-yl]-3-[3-methoxy-4-(4-methyl-1H-imidazol-1-yl)benzylidene]piperidin-2-one or a pharmaceutically acceptable salt thereof; or (E)-1-[(4S)-7-fluorochroman-4-yl]-3-[3-methoxy-4-(4-methyl-1H-imidazol-1-yl)benzylidene]piperidin-2-one or a pharmaceutically acceptable salt thereof;
(E)-3-[3-methoxy-4-(4-methyl-1H-imidazol-1-yl)benzylidene]-1-[(2R)-1,2,3,4-tetrahydronaphthalen-2-yl]piperidin-2-one or a pharmaceutically acceptable salt thereof; (E)-3-[3-methoxy-4-(4-methyl-1H-imidazol-1-yl)benzylidene]-1-[(2S)-1,2,3,4-tetrahydronaphthalen-2-yl]piperidin-2-one or a pharmaceutically acceptable salt thereof; and
(E)-1-[(1R)-1-(2,4-difluorophenyl)ethyl]-3-[3-methoxy-4-(4-methyl-1H-imidazol-1-yl)benzylidene]piperidin-2-one or a pharmaceutically acceptable salt thereof; or (E)-1-[(1S)-1-(2,4-difluorophenyl)ethyl]-3-[3-methoxy-4-(4-methyl-1H-imidazol-1-yl)benzylidene]piperidin-2-one or a pharmaceutically acceptable salt thereof.

(22) The use of (18), wherein (B) is one or more compounds selected from the group consisting of donepezil or a pharmaceutically acceptable salt thereof, huperzine A or a pharmaceutically acceptable salt thereof, tacrine or a pharmaceutically acceptable salt thereof, rivastigmine or a pharmaceutically acceptable salt thereof, galantamine or a pharmaceutically acceptable salt thereof, pramiracetam or a pharmaceutically acceptable salt thereof, aniracetam or a pharmaceutically acceptable salt thereof, nefiracetam or a pharmaceutically acceptable salt thereof, EGb 761 or a pharmaceutically acceptable salt thereof, rosiglitazone or a pharmaceutically acceptable salt thereof, rasagiline or a pharmaceutically acceptable salt thereof, levacecarnine or a pharmaceutically acceptable salt thereof, celecoxib or a pharmaceutically acceptable salt thereof, 3-(2-cyanophenyl)-5-(2-pyridyl)-1-phenyl-1,2-dihydropyridin-2-one, a pharmaceutically acceptable salt thereof or a hydrate thereof, talampanel or a pharmaceutically acceptable salt thereof, becampanel or a pharmaceutically acceptable salt thereof, memantine or a pharmaceutically acceptable salt thereof, neramexane or a pharmaceutically acceptable salt thereof, xaliproden or a pharmaceutically acceptable salt thereof, tarenflurbil or a pharmaceutically acceptable salt thereof, tramiprosate or a pharmaceutically acceptable salt thereof, and leuprorelin-D or a pharmaceutically acceptable salt thereof.

(23) The use of (18), wherein (B) is one or more compounds selected from the group consisting of donepezil or a pharmaceutically acceptable salt thereof, 3-(2-cyanophenyl)-5-(2-pyridyl)-1-phenyl-1,2-dihydropyridin-2-one, a pharmaceutically acceptable salt thereof or a hydrate thereof and memantine or a pharmaceutically acceptable salt thereof.

(24) The use of (18), wherein (B) is donepezil or a pharmaceutically acceptable salt thereof.

(25) The use of (18), wherein (A) and (B) are administered separately to a patient or are administered to a patient in the form of a pharmaceutical composition.

(26) Compounds (A) and (B) for use in the treatment of dementia or one or more mild cognitive impairments; for the prophylaxis of dementia or one or more mild cognitive impairments; or delaying the onset of dementia or one or more mild cognitive impairments, wherein (A) and (B) are:

(A) a compound of Formula (I) or a pharmaceutically acceptable salt thereof:

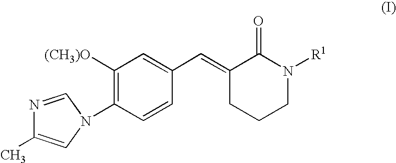

wherein R¹ is:

(a) —X₁—Ar₁, wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

(b) an indenyl group optionally substituted with 1 to 3 halogen atoms;

(c) a tetrahydronaphthyl group optionally substituted with 1 to 3 halogen atoms; or

(d) a chromanyl group optionally substituted with 1 to 3 halogen atoms; and

(B) a cholinesterase inhibitor; an AMPA receptor antagonist; an NMDA receptor antagonist; or a mixture or combination of two or more thereof.

(27) A kit comprising the pharmaceutical composition of any one of (1) to (8) or the combination of any one of (9) to (17).

(28) A method for treating dementia or one or more mild cognitive impairments; for the prophylaxis of dementia or one or more mild cognitive impairments; or delaying the onset of dementia or one or more mild cognitive impairments comprising administering to a patient in need thereof a therapeutically effective amount of the pharmaceutical composition of any one of (1) to (8) or a therapeutically effective amount of the combination of any one of (9) to (17).

These and other aspects of the invention are described in more detail herein.

### DETAILED DESCRIPTION OF THE INVENTION

“Patient” refers to animals, preferably mammals, more preferably humans. The term “patient” includes men and women; and includes adults, children and neonates.

“Active ingredient” refers to and includes compounds useful for treating dementia, delaying the onset of dementia, or the prophylaxis of dementia. The active ingredients may have mechanisms of action that are known or unknown, and the active ingredients may have one or more mechanisms of action. The active ingredient may have an asymmetric carbon depending on the type of substituent and may have a stereoisomer (e.g., a geometric isomer, an enantiomer, a diastereomer or the like). The active ingredient or a stereoisomer thereof may form a pharmaceutically acceptable salt. The active ingredient, a pharmaceutically acceptable salt thereof, a stereoisomer thereof or a pharmaceutically acceptable salt of a stereoisomer thereof may be an anhydride, and may form a solvate. The active ingredient, a pharmaceutically acceptable salt thereof, a stereoisomer thereof, a pharmaceutically acceptable salt of a stereoisomer thereof or a solvate thereof may be a crystal or an amorphous. Crystal polymorph may exist in the active ingredient, a pharmaceutically acceptable salt thereof, a stereoisomer thereof, a pharmaceutically acceptable salt of a stereoisomer thereof or a solvate thereof, although not limited thereto and any form of crystal may exist alone or in combination, which are within the scope of the present invention. Exemplary active ingredients include cinnamide compounds and the second-line compounds described herein.

“Treatment” and “treating” refer to the acquisition of a desired pharmacological effect and/or physiologic effect. These effects are prophylactic in teens of completely or partially preventing a disease and/or symptom(s), and therapeutic in terms of partially or completely curing a disease and/or an adverse event caused by a disease. “Treatment” and “treating” include any treatment of a disease in a patient including, for example: (a) to prevent a disease or symptom(s) in a patient who is suspected of being predisposed to the disease or symptom(s) but not yet diagnosed to be so; (b) to inhibit a symptom(s) of a disease, i.e., to inhibit or delay the progression of the symptom(s); and (c) to alleviate a symptom(s) of a disease, i.e., to reverse or eliminate the symptom(s) of the disease; or to reverse the progress of the symptom(s).

“Administered separately” with reference to the administration of two or more active ingredients to treat and/or prevent and/or delay the onset of the diseases described herein includes, for example, the sequential administration of the active ingredients in any order or the simultaneous administration of the active ingredients. Simultaneous administration of the active ingredients means that the active ingredients are administered to the patient at substantially the same time or at exactly the same time, depending on the mode of administration. The sequential administration of the active ingredients may occur in any order and may occur with any amount of time elapsing between administration of the active ingredients. Sequential administration may be based on factors that would influence which of the active ingredients should be administered first and which should be administered second, and how much time should elapse between administration of the active ingredients. For example, when two or more active ingredients are administered separately and sequentially, factors that effect when the active ingredients are administered to the patient include, for example, (a) the time(s) that provides the best efficacy for the active ingredient being administered, (b) the time(s) that provides the fewest side effects for the active ingredient being administered, (c) the dosage of the active ingredient, (d) the route of administration of the active ingredient, (e) the disease being treated, (f) the patient being treated, (g) the in vivo relationship of the active ingredients being administered, and other such factors known in the art. The time intervals for sequential administration are generally chosen so that the effect on the disease being treated in the combined use of the active ingredients is greater than additive when compared to the effect which would be obtained by use of only one of the active ingredients.

“Combination” refers to two or more active ingredients being administered separately as distinct pharmaceutical formulations (e.g., a first pharmaceutical formulation comprising a cinnamide compound and a second pharmaceutical formulation comprising a second-line active ingredient). The pharmaceutical formulations can have the same or different modes of administration.

“Monotherapy” is a therapy which uses only one active ingredient for treatment and/or prophylaxis and/or delaying the onset of a disease.

“Combination therapy” is a therapy where two or more active ingredients are administered separately or are administered in the form of a pharmaceutical composition for the treatment and/or prophylaxis and/or delayed onset of a disease.

“Therapeutically effective amount” refers to the amount of the active ingredient that is necessary for the treatment and/or prophylaxis and/or delayed onset of a disease. When two or more active ingredients are administered as a pharmaceutical composition or for combination therapy, the term “therapeutically effective amount” refers to the amount of active ingredients that are necessary for treatment and/or prophylaxis and/or delayed onset of a disease and includes, for example: (a) a therapeutically effective amount of a first active ingredient and a therapeutically effective amount of a second active ingredient (i.e., the amount of each active ingredient that would be used for monotherapy for the treatment and/or prophylaxis of a disease is used for the pharmaceutical composition or combination therapy); (b) a therapeutically effective amount of a first active ingredient and a sub-therapeutic amount of a second active ingredient, which in combination effectively provide for treatment and/or prophylaxis of a disease (e.g., the sub-therapeutic amount of the second active ingredient can be used in a pharmaceutical composition or combination therapy to achieve a result that would be equal to or greater than the result that the second active ingredient would achieve if it was used for monotherapy); (c) a sub-therapeutic amount of a first active ingredient and a therapeutically effective amount of a second active ingredient, which in combination effectively provide for treatment and/or prophylaxis of a disease (e.g., the sub-therapeutic amount of the first active ingredient can be used in a pharmaceutical composition or combination therapy to achieve a result that would be equal to or greater than the result that the first active ingredient would achieve if it was used for monotherapy); and (d) a sub-therapeutic amount of a first active ingredient and a sub-therapeutic amount of a second active ingredient, which In combination therapy provide for treatment and/or prophylaxis of a disease or disorder (e.g., the sub-therapeutic amount of the first active ingredient can be used in a pharmaceutical composition or combination therapy to achieve a result that would be equal to or greater than the result that the first active ingredient would achieve if it was used for monotherapy; and the sub-therapeutic amount of the second active ingredient can be used in pharmaceutical composition or combination therapy to achieve a result that would be equal to or greater than the result that the second active ingredient would achieve if it was used for monotherapy).

“Kits” can include a combination of (i) a first pharmaceutical composition or formulation comprising the cinnamide compound; (ii) one or more second-line active ingredients; (iii) instructions for using the pharmaceutical compositions or formulations for treating or preventing or delaying the onset of the disease; and (iv) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months. In another embodiment, “kits” can include (i) pharmaceutical compositions or formulations comprising both the cinnamide compound and one or more second-line active ingredients; (ii) instructions for using the pharmaceutical compositions or formulations for treating or preventing or delaying the onset of the disease; and (iii) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months.

“Solvate” is well known in the art. The solvate is preferably a pharmaceutically acceptable solvate. The pharmaceutically acceptable solvate may be either a hydrate or a nonhydrate, but preferably a hydrate. The solvent such as water, alcohol (e.g., methanol, ethanol, n-propanol), dimethylformamide, dimethyl sulfoxide (DMSO) or the like may be used.

“Hydrate” refers to an active ingredient or compound containing a molecule of water of crystallization. The molecule of water of crystallization can be an integer of 1 or more, such as 1 to 10; or can be any fraction greater than 0 or a fraction of an integer from 1 to 10. For example, the hydrate may be represented as (active ingredient).¼H₂O; (active ingredient).½H₂O; (active ingredient).¼H₂O; (active ingredient).2H₂O; (active ingredient).5½H₂O; (active ingredient).6H₂O; and the like.

“Pharmaceutically acceptable salts” are well known in the art and include those of inorganic acids, such as hydrochloride, sulfate, hydrobromide and phosphate; and those of organic acids, such as formate, acetate, trifluoroacetate, methanesulfonate, benzenesulfonate and toluenesulfonate. When certain substituents are selected, the active ingredients can form, for example, alkali metal salts, such as sodium or potassium salts; alkaline earth metal salts, such as calcium or magnesium salts; organic amine salts, such as a salt with trimethylamine, triethylamine, pyridine, picoline, dicyclohexylamine or N,N′-dibenzylethylenediamine. One skilled in the art will recognize that the active ingredients can be made in the form of any other pharmaceutically acceptable salt. One skilled in the art will also recognize that any active ingredient described herein can be in the form of a pharmaceutically acceptable salt.

“Dementia” refers to a deterioration of intellectual functioning, and is characterized by one or more symptoms of cognitive impairments, disorientation, impaired memory, impaired judgment, impaired intellect, and the like. “Dementia” may also include behavioral disturbances. Exemplary behavioral disturbances include sexual disinhibition, changes in activity, changes in interpersonal relationships, physical aggressiveness, physical non-aggressiveness (e.g., wandering), verbal aggressiveness, and verbal non-aggressiveness (e.g., repetitive vocalization). With respect to dementia, the methods of the invention may be used to treat, prevent, or delay the onset of (i) intellectual functioning associated with dementia; (ii) behavioral disturbances associated with dementia; or (iii) intellectual functioning and behavioral disturbances associated with dementia. In one embodiment, the invention provides methods that are used to treat, prevent, or delay the onset of intellectual functioning associated with dementia. The cause(s) of dementia may be known or unknown.

“Mild cognitive impairments” are a transition, stage between the cognitive changes of normal aging and the more serious problems caused by dementia. While mild cognitive impairments can affect many areas of cognition—language, attention, reasoning, judgment, reading and writing—most research has focused on its effects on memory. The disorder can be divided into two broad subtypes. Amnestic mild cognitive impairments significantly affect memory, while nonamnestic mild cognitive impairments do not. Other functions, such as language and attention span, may be impaired in either subtype.

Exemplary causes of dementia and/or mild cognitive impairments include neurodegenerative diseases, Alzheimer's disease, Parkinson's disease, Huntington's disease, Pick's disease, Lewy body disease, vascular disease (e.g., cerebrovascular disease), HIV/AIDS, epilepsy, brain tumors, brain lesions, multiple sclerosis, Down's syndrome, Rett's syndrome, progressive supranuclear palsy, frontal lobe syndrome, schizophrenia, traumatic brain injuries (e.g., closed head injuries), post coronary artery by-pass graft surgery, electroconvulsive shock therapy, chemotherapy, radiation therapy, radiation exposure, encephalitis, meningitis, fetal alcohol syndrome, Korsakoff's syndrome, anoxic brain injury, cardiopulmonary resuscitation, diabetes, menopause, strokes, high cholesterol levels, or spinal cord disorders. For additional descriptions of dementia and mild cognitive impairments, the disclosures of U.S. Pat. No. 6,458,807, US Publication No. 2006/0018839, and WO 2005/074535 are incorporated by reference herein in their entirety.

“Neurodegenerative disease” refers to any neurodegenerative disease known in the art. Exemplary neurodegenerative diseases include Alzheimer's disease, Parkinson's disease, Huntington's disease, amyotrophic lateral sclerosis, multiple sclerosis, Pick's disease, Lewy body disease, prion diseases (e.g., Creutzfeldt-Jakob disease), epilepsy, strokes, and the like.

“Alzheimer's disease” refers to and includes one or more of mild Alzheimer's disease; mild-to-moderate Alzheimer's disease; moderate Alzheimer's disease; moderate-to-severe Alzheimer's disease; and severe Alzheimer's disease. Clinical symptoms of Alzheimer's disease include progressive disorientation, amnesia, and aphasia, which eventually cause incompetence, speech loss, and akinesia. Examples of pathological signs of Alzheimer's disease include neurofibrillary tangle, senile plaque, and amyloid vascular disorder. “To prevent progression of Alzheimer's disease” refers to preventing the onset or further progression of a clinical symptom(s) and/or a pathological sign(s) of Alzheimer's disease. For example, progression of a clinical symptom or a pathological sign can be prevented for patients who do not exhibit the clinical symptom(s) or pathological sign(s) of Alzheimer's disease. In addition, patients with a milder form of Alzheimer's disease can be prevented from progressing to a more severe of Alzheimer's disease. “To delay the progression of Alzheimer's disease” refers to delaying the onset of a symptom(s) and/or pathological sign(s) of Alzheimer's disease; or to slow down the rate at which Alzheimer's disease progresses, as determined by a clinical symptom(s) and/or a pathological sign(s). “To reverse the progression of Alzheimer's disease” refers to alleviating the severity of a symptom(s) of Alzheimer's disease, i.e., to alter the patient's symptom(s) from a more severe to a more mild condition, as determined by a reduction of the clinical symptom(s) and/or the pathological sign(s).

An Alzheimer's disease diagnosis can be carried out using various known methods. Typically, clinical and pathological assessments are combined to diagnose a patient with Alzheimer's disease. For example, progression or severity of Alzheimer's disease can be assessed using the Mini Mental State Examination (MMSE) (Mohs et al, (1996) Int. Psychogeriatr 8:195-203); the Alzheimer's Disease Assessment Scale-Cognitive Subscale (ADAS-cog) (Galasko et al, (1997) Alzheimer Dis. Assoc. Disord., 11 Suppl. 2:S33-39); the Alzheimer's Disease Cooperative Study-Activities of Daily Living scale (ADCS-ADL) (McKhann et al, (1984) Neurology 34:939-944); and the National Institute of Neurologic Communicative Disorders and the Stroke-Alzheimer's Disease and Related Disorders Association (NINCDS-ADRDA) criteria (Folstein et al, (1975) J. Psychiatr. Res. 12:189-198; McKhann et al, (1984) Neurology 34:939-944). Additionally, methods capable of assessing various areas in the brain to estimate frequency of senile plaque or neurofibrillary tangle may be used (Braak et al (1991) Acta Neuropathol 82:239-259; Khachaturian (1985) Arch Neuro 42:1097-1105; Mirra et al, (1991) Neurology, 41:479-486; Mirra et al, (1993) Arch Pathol Lab Med 117:132-144).

The invention provides methods for treating dementia and/or mild cognitive impairments; methods for the prophylaxis of dementia and/or mild cognitive impairments; and methods for delaying the onset of dementia and/or mild cognitive impairments in a patient in need thereof by administering at least one cinnamide compound and one or more second-line active ingredients selected from the group consisting of cholinesterase inhibitors (e.g., donepezil, huperzine A, tacrine, rivastigmine, galantamine); AMPA receptor antagonists (e.g., 1,2-dihydropyridine compounds such as 3-(2-cyanophenyl)-5-(2-pyridyl)-1-phenyl-1,2-dihydropyridin-2-one); and NMDA receptor antagonists (e.g., memantine).

The invention provides methods for treating dementia and/or mild cognitive impairments; methods for the prophylaxis of dementia and/or mild cognitive impairments; and methods for delaying the onset of dementia and/or mild cognitive impairments in a patient in need thereof by administering at least one cinnamide compound and one or more second-line active ingredients selected from the group consisting of cholinesterase inhibitors (e.g., donepezil, huperzine A, tacrine, rivastigmine, galantamine); AMPA receptor antagonists (e.g., 1,2-dihydropyridine compounds such as 3-(2-cyanophenyl)-5-(2-pyridyl)-1-phenyl-1,2-dihydropyridin-2-one); and NMDA receptor antagonists (e.g., memantine); pramiracetam; aniracetam; acetylcholine releasing stimulants (e.g., nefiracetam); calcium channel agonists (e.g., nefiracetam); free radical scavengers (e.g., EGb 761); platelet activating factor antagonists (e.g., EGb 761); platelet aggregation antagonists (e.g., EGb 761, triflusal); insulin sensitizers (e.g., rosiglitazone); peroxisome proliferator-activated receptor agonists (e.g., rosiglitazone); peroxisome proliferator-activated receptor gamma agonists (e.g., rosiglitazone); monoamine oxidase B inhibitors (e.g., rasagiline, selegiline, procaine); carnitine acetyltransferase stimulants (e.g., levacecarnine); NSAIDs (e.g., triflusal, cyclooxygenase-2 inhibitors, such as celecoxib); nerve growth factor agonists (e.g., xaliproden, FPF 1070); beta-amyloid inhibitors (e.g., tarenflurbil, tramiprosate, leuprorelin-D); immunomodulators (e.g., tarenflurbil, immune globulin, icosapentethyl ester); and NF-kappa B inhibitors (e.g., tarenflurbil).

The invention provides methods for treating dementia and/or mild cognitive impairments; methods for the prophylaxis of dementia and/or mild cognitive impairments; and methods for delaying the onset of dementia and/or mild cognitive impairments in a patient in need thereof by administering at least one cinnamide compound and one or more second-line active ingredients selected from the group consisting of cholinesterase inhibitors (e.g., donepezil, huperzine A, tacrine, rivastigmine, galantamine); AMPA receptor antagonists (e.g., 1,2-dihydropyridine compounds such as 3-(2-cyanophenyl)-5-(2-pyridyl)-1-phenyl-1,2-dihydropyridin-2-one); and NMDA receptor antagonists (e.g., memantine); pramiracetam; aniracetam; acetylcholine releasing stimulants (e.g., nefiracetam); calcium channel agonists (e.g., nefiracetam); free radical scavengers (e.g., EGb 761); platelet activating factor antagonists (e.g., EGb 761); platelet aggregation antagonists (e.g., EGb 761, triflusal); insulin sensitizers (e.g., rosiglitazone); peroxisome proliferator-activated receptor agonists (e.g., rosiglitazone); peroxisome proliferator-activated receptor gamma agonists (e.g., rosiglitazone); monoamine oxidase B inhibitors (e.g., rasagiline, selegiline, procaine); carnitine acetyltransferase stimulants (e.g., levacecarnine); NSAIDs (e.g., triflusal, cyclooxygenase-2 inhibitors, such as celecoxib); nerve growth factor agonists (e.g., xaliproden, FPF 1070); beta-amyloid inhibitors (e.g., tarenflurbil, tramiprosate, leuprorelin-D); immunomodulators (e.g., tarenflurbil, immune globulin, icosapentethyl ester); NF-kappa B inhibitors (e.g., tarenflurbil); thyrotropin releasing hormone agonists (e.g., taltirelin); dopamine D2 receptor antagonists (e.g., risperidone); serotonin 2 receptor antagonists (e.g., risperidone); muscarinic M1 receptor agonists (e.g., cevimeline); alpha 1 adrenoceptor agonists (e.g., modafinil); serotonin 3 receptor antagonists (e.g., alosetron); dopamine D2 receptor agonists (e.g., aripiprazole); dopamine D2 receptor antagonists (e.g., aripiprazole); serotonin 1A receptor agonists (e.g., aripiprazole); serotonin 2A receptor antagonists (e.g., aripiprazole); glucocorticoid antagonists (e.g., mifepristone); progesterone antagonists (e.g., mifepristone); HMG-CoA reductase inhibitors (e.g., atorvastatin, simvastatin); adenosine uptake inhibitors (e.g., propentofylline); phosphodiesterase inhibitors (e.g., propentofylline); acetylcholine receptor agonists (e.g., choline alfoscerate); membrane permeability enhancers (e.g., choline alfoscerate); cannabinoid 1 receptor antagonists (e.g., rimonabant); cannabinoid receptor agonists (e.g., dronabinol); angiogenesis inhibitors (e.g., paclitaxel); immunosuppressants (e.g., paclitaxel); tubulin antagonists (e.g., paclitaxel); thromboxane A2 synthase inhibitors (e.g., triflusal); antioxidants (e.g., idebenone); alpha adrenoreceptor antagonists (e.g., nicergoline); estrogen agonists (e.g., conjugated estrogens, trilostane); 3-beta hydroxysteroid dehydrogenase inhibitors (e.g., trilostane); signal transduction pathway inhibitors (e.g., trilostane); melatonin receptor agonists (e.g., ramelteon); immunostimulants (e.g., immune globulin, icosapentethyl ester, procaine); HIV entry inhibitors (e.g., procaine); sodium channel antagonists (e.g., procaine); microtubule inhibitor (e.g., CPH 82); glycine NMDA agonists (e.g., cycloserine); adenosine A1 receptor antagonists (e.g., KW 3902); ATPase stimulants (e.g., triacetyluridine); mitochondrial function enhancers (e.g, triacetyluridine); and growth hormone releasing factor agonists (e.g., tesamorelin).

The invention provides methods for treating dementia and/or mild cognitive impairments; methods for the prophylaxis of dementia and/or mild cognitive impairments; and methods for delaying the onset of dementia and/or mild cognitive impairments in a patient in need thereof by administering at least one cinnamide compound and one or more second-line active ingredients selected from the group consisting of donepezil, 3-(2-cyanophenyl)-5-(2-pyridyl)-1-phenyl-1,2-dihydropyridin-2-one, and memantine.

The invention provides methods for treating dementia and/or mild cognitive impairments; methods for the prophylaxis of dementia and/or mild cognitive impairments; and methods for delaying the onset of dementia and/or mild cognitive impairments in a patient in need thereof by administering at least one cinnamide compound and one or more second-line active ingredients selected from the group consisting of donepezil, huperzine A, tacrine, rivastigmine, galantamine, pramiracetam, aniracetam, nefiracetam, EGb 761, rosiglitazone, rasagiline, levacecarnine, celecoxib, 3-(2-cyanophenyl)-5-(2-pyridyl)-1-phenyl-1,2-dihydropyridin-2-one, talampanel, becampanel, memantine, neramexane, xaliproden, tarenflurbil, tramiprosate, and leuprorelin-D.

The invention provides pharmaceutical compositions comprising a therapeutically effective amount of at least one cinnamide compound and one or more second-line active ingredients selected from the group consisting of cholinesterase inhibitors; AMPA receptor antagonists; and NMDA receptor antagonists. The pharmaceutical compositions may comprise one or more pharmaceutically acceptable carriers.

The invention provides pharmaceutical compositions comprising a therapeutically effective amount of at least one cinnamide compound and one or more second-line active ingredients selected from the group consisting of cholinesterase inhibitors; AMPA receptor antagonists; NMDA receptor antagonists; pramiracetam; aniracetam; acetylcholine releasing stimulants; calcium channel agonists; free radical scavengers; platelet activating factor antagonists; platelet aggregation antagonists; insulin sensitizers; peroxisome proliferator-activated receptor agonists; peroxisome proliferator-activated receptor gamma agonists; monoamine oxidase B inhibitors; carnitine acetyltransferase stimulants; NSAIDs; nerve growth factor agonists; beta-amyloid inhibitors; immunomodulators; and NF-kappa B inhibitors. The pharmaceutical compositions may comprise one or more pharmaceutically acceptable carriers.

The invention provides pharmaceutical compositions comprising a therapeutically effective amount of at least one cinnamide compound and one or more second-line active ingredients selected from the group consisting of cholinesterase inhibitors; AMPA receptor antagonists; NMDA receptor antagonists; pramiracetam; aniracetam; acetylcholine releasing stimulants; calcium channel agonists; free radical scavengers; platelet activating factor antagonists; platelet aggregation antagonists; insulin sensitizers; peroxisome proliferator-activated receptor agonists; peroxisome proliferator-activated receptor gamma agonists; monoamine oxidase B inhibitors; carnitine acetyltransferase stimulants; NSAIDs; nerve growth factor agonists; beta-amyloid inhibitors; immunomodulators; NF-kappa B inhibitors; thyrotropin releasing hormone agonists; dopamine D2 receptor antagonists; serotonin 2 receptor antagonists; muscarinic M1 receptor agonists; alpha 1 adrenoceptor agonists; serotonin 3 receptor antagonists; dopamine D2 receptor agonists; dopamine D2 receptor antagonists; serotonin 1A receptor agonists; serotonin 2A receptor antagonists; glucocorticoid antagonists; progesterone antagonists; HMG-CoA reductase inhibitors; adenosine uptake inhibitors; phosphodiesterase inhibitors; acetylcholine receptor agonists; membrane permeability enhancers; cannabinoid 1 receptor antagonists; cannabinoid receptor agonists; angiogenesis inhibitors; immunosuppressants; tubulin antagonists; thromboxane A2 synthase inhibitors; antioxidants; alpha adrenoreceptor antagonists; estrogen agonists; 3-beta hydroxysteroid dehydrogenase inhibitors; signal transduction pathway inhibitors; melatonin receptor agonists; immunostimulants; HIV entry inhibitors; sodium channel antagonists; microtubule inhibitors; glycine NMDA agonists; adenosine A1 receptor antagonists; ATPase stimulants; mitochondrial function enhancers; and growth hormone releasing factor agonists. The pharmaceutical compositions may comprise one or more pharmaceutically acceptable carriers.

The invention provides pharmaceutical compositions comprising a therapeutically effective amount of at least one cinnamide compound and one or more second-line active ingredients selected from the group consisting of donepezil, 3-(2-cyanophenyl)-5-(2-pyridyl)-1-phenyl-1,2-dihydropyridin-2-one, and memantine. The pharmaceutical compositions may comprise one or more pharmaceutically acceptable carriers.

The invention provides pharmaceutical compositions comprising a therapeutically effective amount of at least one cinnamide compound and one or more second-line active ingredients selected from the group consisting of donepezil, huperzine A, tacrine, rivastigmine, galantamine, pramiracetam, aniracetam, nefiracetam, EGb 761, rosiglitazone, rasagiline, levacecarnine, celecoxib, 3-(2-cyanophenyl)-5-(2-pyridyl)-1-phenyl-1,2-dihydropyridin-2-one, talampanel, becampanel, memantine, neramexane, xaliproden, tarenflurbil, tramiprosate, and leuprorelin-D. The pharmaceutical compositions may comprise one or more pharmaceutically acceptable carriers.

The invention provides pharmaceutical compositions comprising a therapeutically effective amount of at least one cinnamide compound and one or more second-line active ingredients selected from the group consisting of donepezil, huperzine A, tacrine, rivastigmine, galantamine, pramiracetam, aniracetam, nefiracetam, EGb 761, rosiglitazone, rasagiline, levacecarnine, celecoxib, 3-(2-cyanophenyl)-5-(2-pyridyl)-1-phenyl-1,2-dihydropyridin-2-one, talampanel, becampanel, memantine, xaliproden, tarenflurbil, tramiprosate, leuprorelin-D, taltirelin, risperidone, cevimeline, modafinil, alosetron, aripiprazole, mifepristone, atorvastatin, propentofylline, choline alfoscerate, FPF 1070 (CAS Number 143637-01-8), rimonabant, dronabinol, docosahexaenoic acid, paclitaxel, triflusal, idebenone, nicergoline, conjugated estrogens, trilostane, simvastatin, selegiline, ramelteon, immune globulin, icosapentethyl ester, procaine, CPH 82, cycloserine, KW 3902 (CAS Number 136199-02-5), triacetyluridine, estrogen dementia therapeutics (e.g., MIGENIX, Vancouver, Canada), and tesamorelin. In other embodiments, the second-line active ingredients can be one or more compounds selected from the group consisting of levprorelin, prasterone, peptide T (CAS Number 53-43-0), besipiridine, lexipafant, stacofylline, SGS 742 (CAS Number 123690-78-8), T 588 (CAS Number 142935-03-3), nerispiridine, dexanabinol, sabcomeline, GTS 21 (CAS Number 156223-05-1), CX 516 (CAS Number 154235-83-3), ABT 089 (CAS Number 161417-03-4), anapsos, tesofensine, SIB 1553A (i.e., 4-[[2-(1-methyl-yl-2-pyrrolidinyl)ethyl]thia]phenol), ladostigil, radequinil, GPI 1485, ispronicline, arundic acid, MEM 1003 (i.e., 3-Isopropyl 5-(2-methoxyethyl) 4-(2-chloro-3-cyanophenyl)-2,6-dimethylpyridine-3,5-dicarboxylate), V 3381 (i.e., 2-(2,3-Dihydro-1H-inden-3-ylamino)acetamide hydrochloride), farampator, paliroden, prasterone-paladin, urocortin, DP b99 (i.e., 2,2′-(Ethylenedioxy)bis(2,1-phenylene)bis[N-[2-[2-(octyloxy)ethoxy]-2-oxoethyl]imino]bis(acetic acid)), capserod, DU 125530, bapineuzumab, AL 108 (i.e., L-Asparaginyl-L-alanyl-L-prolyl-L-valyl-L-seryl-L-isoleucyl-L-prolyl-L-glutamine), DAS 431, DEBIO 9902, DAR 100, mitoquinone, IPL 455903 (i.e., 5(S)-[3-(Cyclopentyloxy)-4-methoxyphenyl]-3(S)-(3-methylbenzyl)piperidin-2-one), E2CDS, PYM 50028, PBT 2, lecozotan, SB 742457, CX 717, AVE 1625 (i.e., 1-(bis(4-chlorophenyl)methyl)-3-((3,5-difluorophenyl)(methylsulfonyl)methylene)azetidine), LY 450139 (i.e., N2-[2(s)-Hydroxy-3-methylbutyryl]-N1-[3-methyl-2-oxo-2,3,4,5-tetrahydro-1H-3-benzazepin-1(5)-yl]-L-alaninamide), EM 1421 (i.e., 4,4′-[(2R,3S)-2,3-Dimethylbutane-1,4-diyl]bis(1,2-dimethoxybenzene), SRN 001, TTP 488, PRX 03140, dimebolin, glycine-proline-glutamate, C105, AL 208, MEM 3454, AC 1202, L 830982, LY 451395 (i.e., (R)—N-[2-[4′-(methylsulfonamidomethyl)biphenyl-4-yl]propyl]propane-2-sulfonamide), MK 0249, LY 2062430, diethylnorspermine, neboglamine, S 18986, SA 4503 (CAS Number 165377-44-6), GRI 1, S 17092 (i.e., (2S,3aS,7aS)-1{[(R,R)-2-Phenylcyclopropyl]carbonyl}-2-[(thiazolidin-3-yl)carbonyl]octahydro-1H-indole), SL 251188, EUK 189, R 1450, 6,6-dimethyl-3-(2-hydroxyethyl)thio-1-(thiazol-2-yl)-6,7-dihydro-2-benzothiophen-4(5H)-one, CERE 110, dexefaroxan, CAD 106, HF 0220, HF 0420, EHT 0202, VP 025, MEM 1414, BGC 201259 (i.e., N,N-Dimethylcarbamic acid, 4-[1(S)-(methylamino)-3-(4-nitrophenoxy)propyl]phenyl ester), EN 100, ABT 834, ABT 239 (i.e., 4-[2-[2-[(2R)-2-Methylpyrrolidinyl]ethyl]-benzofuran-5-yl]benzonitrile), SGS 518, R 1500, C 9138, SSR 180711, alfatradiol, R 1577, T 817MA (i.e., 1-[3-[2-(1-Benzothien-5-yl)ethoxy]propyl]azetidin-3-olmaleate), CNP 1061 (i.e., 4-Methyl-5-(2-nitrooxyethyl)thiazole), KTX 0101 (i.e., sodium beta-hydroxybutyrate), GSK 189254 (i.e., 6-[3-Cyclobutyl-2,3,4,5-tetrahydro-1H-benzo[d]azepin-7-yloxy]-N-methylnicotinamide), AZD 1080, ACC 001, PRX 07034, midazolam, R-phenserine, AZD 103 (CAS Number 488-59-5), SN 522, NGX 267 (CAS Number 503431-81-0), N-PEP-12, RN 1219, FGLL, AVE 8112, EVT 101, NP 031112, MK 0752, MK 0952, LX 6171, PAZ 417, AV 965, PF 3084014, SYN 114, GSI 953, SAM 315, SAM 531, D-serine, leteprinim potassium, BR 16A (CAS Number 149175-77-9), RPR 107393 (CAS Number 190841-57-7), NXD 2858, REN 1654, CDD 0102, NC 1900 (CAS Number 132925-74-7), ciclosporin, NCX 2216 (i.e., (E)-4-(Nitrooxy)butyl 3-[4-[2-(2-fluorobiphenyl-4-yl)propanoyloxy]-3-methoxyphenyl]acrylate), NXD 3109, NXD 1191, ZSET 845 (i.e., 3,3-diphenylimidazo[1,2-a]pyridin-2-(3H)-one), ET 002, NT 13, RO 638695 (i.e., [1,6-(1,6-dioxohexyl)]dipyrrolidine-(2R)-carboxylic acid), bisnorcymserine, BA 1016, XD 4241, EUK 207 (i.e., (SP-5-13)-(acetato-κO)[13,16,19,22-tetraoxa-3,6-diazatricyclo[21.3.18.12]octacosa-1(27),2,6,8,10,12(28),23,25-octaene-27,28-diolato(2-)-κN3,κN6,κO27,κO28]manganese), LG 617 inhibitors, ZSET 1446, PAN 811, F 14413 (i.e., 2-[5-fluoro-2(S)-methoxy-2,3-dihydro-1,4-benzodioxin-2-yl]-4,5-dihydro-1H-imidazole), FP 7832 (i.e., N-[2-(5-methoxy-1-nitroso-1H-indol-3-yl)ethyl]acetamide), ARA 014418 (i.e., N-(4-methoxybenzyl)-N′-(5-nitro-1,3-thiazol-2-yl)urea), AZD 3102, KP 544 (i.e., 2-amino-5-(4-chlorophenylethynyl)-4-(4-trans-hydroxycyclohexylamino)pyrimidine), DP 155, 5-chloro-N-[3-[2-(dimethylamino)ethyl]-1H-indol-5-yl]naphthalene-2-sulfonamide, TAK 070, huperzine, N-[2-(3,5-dimethyladamant-1-yl)ethyl]acetamidine hydrochloride, 6-[4-[(dimethylamino)methyl]-5-ethyl-2-methoxyphenyl]pyridin-2-amine, 4,6-diphenyl-3-(4-(pyrimidin-2-yl)piperazin-1-yl)pyridazine, N-[(1S,2R)-3-(3,5-difluorophenyl)-1-hydroxy-1-[(5S,6R)-5-methyl-6-(neopentyloxy)morpholin-3-yl]propan-2-yl]acetamide hydrochloride, N-[(1R,25)-3-(3,5-difluorophenyl)-1-hydroxy-1-[(2R,4R)-4-phenoxypyrrolidin-2-yl]propan-2-yl]-3-[(R)-2-(methoxymethyl)pyrrolidine-1-carbonyl]-5-methylbenzamide, R 1589, midafotel, phenserine, coluracetam, physostigmine, cipralisant, nitroflurbiprofen, PPI 1019 (i.e., (3α,5β,7α,12α)-trihydroxycholan-24-oyl-L-leucyl-L-valyl-L-phenylalanyl-L-phenylalanyl-L-alanine), dapsone, MDL 100453 (CAS Number 129938-34-7), NS 377, midaxifylline, propofol phosphate, metrifonate, ceronapril, tenilsetam, sufoxazine, seglitide, ebiratide, nebracetam, milacemide, iododoxorubicin, SM 10888 (CAS Number 129297-21-8), U 80816 (CAS Number 138554-11-7), YM 954 (CAS Number 132041-85-1), SUT 8701 (CAS Number 123577-73-1), apovincamine, FR 121196 (CAS Number 133920-65-7), LY 274614 (CAS Number 136109-04-1), CL 275838 (CAS Number 115931-65-2), igmesine, K 7259 (CAS Number 133667-88-6), vinconate, itasetron, CL 287663 (CAS Number 125109-98-0), WAY 100289 (CAS Number 136013-69-9), SR 46559A (CAS Number 137733-33-6), GYKI 46903 (CAS Number 142999-59-5), L 670548 (CAS Number 121564-89-4), Y 29794 (CAS Number 129184-48-1), AF 125 (CAS Number 7631-86-9), KFM 19 (CAS Number 133058-72-7), ST 796 (i.e., (S)-3-[3-(trifluoromethyl)benzoyl)amino]hexahydroazepin-2-one), RU 33965 (CAS Number 122321-05-5), SDZ 210086 (i.e., (−)-1′,2(S)-Dimethylspiro[1,3-dioxolan-4,4′-piperidine]), L 689660 (CAS Number 144860-79-7), L 689560 (CAS Number 139051-78-8), ST 618 (i.e., 1-(6,7-Dimethoxy-1,2,3,4-tetrahydro-2-naphthyl)-4-hydroxy pyrrolidin-2-one), U 74500A (CAS Number 110101-65-0), GEA 857 (CAS Number 120493-42-7), BIBN 99 (CAS Number 145301-48-0), DX 9366, ONO 1603 (CAS Number 114668-76-7), MDL 102234 (CAS Number 137766-81-5), P 9939 (CAS Number 157971-37-4), PD 140532 (CAS Number 157971-39-6), azetirelin, MR 16728 (CAS Number 147614-21-9), dabelotine, MDL 102503 (i.e., 8-[1(R)-methyl-2-phenylethyl]-1,3-dipropyl-7H-xanthine), PD 141606 (i.e., (±)-(Z)-3-(3-Phenyl-2-propynyloxyimino)-1-azabicyclo[2.2.1]heptane), SNK 882 (CAS Number 152221-12-0), L 696986 (CAS Number 141553-45-9), tazomeline, LY 235959 (CAS Number 137433-06-8), 2-(2-thiooxopyrrolidin-1-yl)acetamide, AK 30 NGF, ABT 418 (CAS Number 147402-53-7), itameline, HUP 13, sibopirdine, KST 5452 (CAS Number 157998-88-4), TJ 54, U 92798 (i.e., 7-[4-[Bis(4-fluorophenyl)methyl]perhydro-1,4-diazepin-1-ylmethyl]-4-isopropyl-2-methoxy-2,4,6-cycloheptatrien-1-one), U 92032 (CAS Number 142223-92-5), 3-(sulfamoyloxy)estra-1,3,5(10)-trien-17-one, P 11012 (CAS Number 164723-36-8), A 82695 (CAS Number 147388-86-1), FR 76659 (CAS Number 116904-25-7), apaxifylline, CX 417, 7 MEOTA (CAS Number 5778-80-3), BU 4514N (CAS Number 151013-39-7), pregnenolone, mexidol, ST 857 (CAS Number 154755-63-2), RU 49041 (CAS Number 123828-80-8), RU 35929 (CAS Number 111711-47-8), P 878184, P 128 (CAS Number 157716-52-4), eurystatin A, eurystatin B, LK 12, NBI 108, NBI 107, NBI 117, L 705106, bacoside A+B, clausenamide, SM 21 (CAS Number 155156-22-2), alaptide, RS 17017 (i.e., 1-(4-Amino-5-chloro-2-methoxyphenyl)-5-(1-piperidinyl)-1-pentanone hydrochloride), AF 150(S) (i.e., (S)-[1-Methyl-piperidine-4-spiro-(2′-methylthiazoline)]), RO 153505 (CAS Number 78771-13-8), PV 113 (i.e., 1,2,3,4-Tetrahydropyrrolo-[1,2-a]-pyrazine), arisugacin, A 98284 (i.e., 2(R)-(3-Methylisoxazol-5-yl) quinuclidine), AP 5 (CAS Number 136941-85-0), BD 1054, SDZ NDD 094 (i.e., bis-(2-(2-methylimidazol-1-yl]methyl)-pyridine-tris(hydrogen-fumarate), AZ 36041 (CAS Number 173324-76-0), quilostigmine, A 84543 (i.e., 3-[1-Methylpyrrolidin-2-(S)-ylmethoxy]pyridine fumarate), BTG 4247 (i.e., (2-[2-Chloroethoxy[4-(dimethylamino)phenyl]phosphoryl]-acetohydrazide), CGP 50068 (CAS Number 158647-49-5), cerebrocrast, desferri-nordanoxamine, isolichenan, MHP 133 (i.e., 3-(N,N-dimethylcarbamoyloxy)-1-methyl-2-(4-phenyl-semicarbazonomethyppyridinium chloride), FR 152558 (CAS Number 151098-08-7), GVS 111 (CAS Number 157115-85-0), P 11149 (CAS Number 164724-79-2), PDC 008004, KST 2818 (CAS Number 158623-26-8), KST 5410 (CAS Number 158623-27-9), RU 52583 (CAS Number 123829-33-4), PD 151832 (CAS Number 149929-39-5), UCL 1199 (i.e., 4-[2-[(5-Nitropyridin-2-ylsulfanyl)ethyl]-1H-imidazole), isovanihuperzine A, SIB 1765F (CAS Number 179120-52-6), JWS USC 751X (i.e., 3-[[[2-[[(5-dimethylaminomethyl)-2-furanyl]methyl]thio]ethyl]amino]-4-nitropyridazine), GR 175737 (i.e., 3-(4-Chlorobenzyl)-5-[2-(1H-imidazol-4-yl)ethyl]-1,2,4-oxadiazole), KS 505A (CAS Number 131774-53-3), ZTTA 1 (i.e., N-benzyloxycarbonyl-thioprolyl-thioprolynal-dimethylaceta 1), AGN 190837 (CAS Number 136527-40-7), P 10358 (188240-59-7), WAY 131256 (CAS Number 174001-71-9), DBO 83 (i.e., 3-(6-chloropyridazin-3-yl)-diazabicyclo[3.2.1]octane dihydrochloride monohydrate), FUB 181 (CAS Number 152029-80-6), RJR 2557, WSU 2088, LVV-haemorphin-7, M 40 (i.e., galanin[1-12]-Pro3-(Ala-Leu)₂-Ala-NH₂), SIB 1757, SKF 74652 (i.e., [5-chloro-2-(4-methoxy phenyl)-3-benzofuranyl][4-[3-(diethylamino)-propoxy]phenyl]methanone), CGP 71982, SCH 57790 (i.e., 4-cyclohexyl-alpha-[4-[[4-methoxyphenyl]sulfinyl]phenyl]-1-piperazineacetonitrile), Putrescine-D-YiAbeta11, DU 14 (i.e., p-O-(sulfamoyl)-N-tetradecanoyl tyramine), CLZ 4, SL 340026, PPRT 424, ciproxifan, UR 1827 (i.e., 2-(1-benzylpiperidin-4-yl)-1-[4-(5-methylpyrimidin-4-ylamino)phenyl]-1-ethanone), caproctamine, TGS 20 (i.e., L-pyroglutamil-D-alanine amide), PG 9 (i.e., alpha-tropanyl 2-[(4-bromo)phenyl]propionate), TEI 3356 (i.e., (16S)-15-Deoxy-16-hydroxy-16-methyl-9-(O)-methano-DELTA6(9alpha)-prostaglandin 11), LY 392098 (i.e., Thiophene, 3-[(2-methylethyl-2)sulphonylaminopropyl-2]phenyl-4-yl-), PG 1000, DM 232, NEPP 11 (i.e., 12-iso-15-Deoxy-18-(4-methyl)phenyl-13,14-dihydro-delta7-prostaglandinA1 methyl ester), VA 100 (i.e., (2,3-Dihydro-2-[[(4-fluorobenzoyl)amino]ethyl]-1-methyl-5-phenyl-1H-1,4-benzodiazepine), VA 101 (i.e., (2,3-dihydro-2-[[(2-thienylcarbonyl)amino]ethyl]-1-methyl-5-phenyl-1H-1,4-benzodiazepine), NC 111585 (i.e., (3S)-1,3-Bis-[3-[(3-azabicylo[2.2.2]octanyl)-1,2,5-thiadiazol-4-yloxy]-1-propyn-1-yl]benzene, 2L-(+)-tartate), IN 201, imoproxifan, kanokodiol, picroside I, picroside II, DM 235 (i.e., 1-(4-Benzoylpiperazin-1-yl)propan-1-one), monoclonal antibody 10D5, JLK2, JLK 6, JLK 7, DAPT (i.e., N-[N-(3,5-difluorophenacetyl)-L-alanyl]-S-phenylglycine t-butyl ester), huperine X, SGS 111 (i.e., (S)-ethyl 2-[1-(2-phenylacetyl)pyrrolidine-2-carboxamido]acetate), NP 7557, C 9136, C 7617, R 1485, rofecoxib, velnacrine, montirelin, lazabemide, ORG 2766 (CAS Number 50913-82-1), sabeluzole, adafenoxate, CAS Number 9061-61-4, ipidacrine, bemesetron, idazoxan, linopirdine, selfotel, suritozole, milameline, xanomeline, TJ 960, fasoracetam, eptastigmine, ensaculin, zanapezil, posatirelin, zacopride, RS 86 (CAS Number 3576-73-6), ORG 5667 (CAS Number 37552-33-3), RX 77368 (CAS Number 76820-40-1), BMS 181168 (CAS Number 123259-91-6), BY 1949 (CAS Number 90158-59-1), AWD 5239 (CAS Number 109002-93-9), YM 796 (171252-79-2), aloracetam, CI-933 (CAS Number 91829-95-7), ST 793 (CAS Number 99306-37-3), cebaracetam, zifrosilone, talsaclidine, alvameline, JTP 2942 (148152-77-6), OPC 14117 (CAS Number 103233-65-4), elziverine, AP 521 (i.e., N-(1,3-Benzodioxol-5-ylmethyl)-1,2,3,4-tetrahydro[1]benzothieno[2,3-c]pyridine-3(R)-carboxamide hydrochloride), S 8510 (CAS Number 151466-23-8), JTP 4819 (CAS Number 162203-65-8), icopezil, SC 110, FK 960 (CAS Number 133920-70-4), DMP 543 (CAS Number 160588-45-4), ganstigmine, CI 1017 (i.e., (R)-(−)-(Z)-1-Azabicyclo[2.2.1]heptan-3-one, 0-(3-(3′-methoxyphenyl)-2-propynyl)-oxime maleate), T 82 (i.e., 2-[2-(1-Benzylpiperidin-4-yl)ethyl]-2,3-dihydro-9-methoxy-1H-pyrrolo[3,4-b]quinolin-1-one hemifumarate), NGD 971, vaccine of Aspartyl-alanyl-glutamyl-phenylalanyl-arginyl-histidyl-aspartyl-seryl-glycyl-tyrosyl-glutamyl-valyl-histidyl-histidyl-glutaminyl-lysyl-leucyl-valyl-phenylalanyl-phenylalanyl-alanyl-glutamyl-aspartyl-valyl-glycyl-seryl-asparaginyl-lysyl-glycyl-alanyl-isoleucyl-isoleucyl-glycyl-leucyl-methionyl-valyl-glycyl-glycyl-valyl-valyl-isoleucyl-alanine, PBT 1 (CAS Number 130-26-7), TCH 346, FK 962 (i.e., N-(1-acetylpiperidin-4-yl)-4-fluorobenzamide), voxergolide, KW 6055 (CAS Number 63233-46-5), thiopilocarpine, ZK 93426 (CAS Number 89592-45-0), SDZ NVI 085 (CAS Number 104195-17-7), CI 1002 (CAS Number 149028-28-4), Z 321 (CAS Number 130849-58-0), mirisetron, CHF 2060 (i.e., N-Heptylcarbamic acid 2,4a,9-trimethyl-2,3,4,4a,9,9a-hexahydro-1,2-oxazino[6,5-b]indol-6-yl ester-L-tartrate), gedocarnil, terbequinil, HOE 065 (CAS Number 123060-44-6), SL 650102, GR 253035, ALE 26015, SB 271046 (i.e., 5-Chloro-N-(4-methoxy-3-piperazin-1-yl-phenyl)-3-methyl-2-benzothiophenesulfonamide), iAbeta5, SCH 211803 (i.e., Piperidine, 1-[1-(3-methyl-2-aminophenyl)carbonylpiperidin-4-yl]-4-[(3-chlorophenypsulphonylphenyl-4]methyl-), EVT 301, alpha-Linolenic acid/linoleic acid, Kamikihi-To, siagoside, FG 7142 (CAS Number 78538-74-6), RU 47067 (CAS Number 111711-92-3), RU 35963 (CAS Number 139886-03-6), FG 7080 (CAS Number 100332-18-1), E 2030 (CAS Number 142007-70-3), transforming growth factor beta-1, A 72055 (i.e., 2′,1-Dimethylspiro[piperidine-4,5′ oxazolidine]-3′-carboxaldehyde), NS 626, dimiracetam, GT 3001, GT 2501, GT 2342, GT 2016 (CAS Number 152241-24-2), ORG 20091 (CAS Number 141545-50-8), BCE 001 (CAS Number 95678-81-2), CGP 35348 (CAS Number 123690-79-9), WAY 100635 (CAS Number 146714-97-8), E 4804 (CAS Number 162559-34-4), LIGA 20 (CAS Number 126586-85-4), NG 121 (i.e., 2-[4,8-Dimethyl-3(E),7(E)-nonadienyl]-3,5-dihydroxy-2-methyl-3,4,7,9-tetrahydro-2H-furo[3,4-13]-1-benzopyran-7-one), MF 247 (i.e., N-[10-(Diethylamino)decyl]carbamic acid (3aS,8aR)-1,3a,8-trimethyl-1,2,3,3a,8,8a-hexahydropyrrolo[2,3-b]indol-5-yl ester), JTP 3399 (i.e., N-Benzyl-2(S)-[2(S)-(phenoxyacetyl)pyrrolidin-1-ylcarbonyl]pyrrolidine-1-carboxamide), KF 17329, thioperamide, F 3796 (i.e., 1-[2-(1-Benzylpiperidin-4-yl)ethyl]-3-[3,4-(methylene-dioxy)benzoyl]thiourea), GT 4001, GT 4002, FPL 14995 (CAS Number 123319-03-9), RU 34332 (CAS Number 137157-58-5), SR 96777A (CAS Number 115767-94-7), SIB T1980, NS 649 (CAS Number 146828-02-6), PD 142505 (CAS Number 149929-08-8), GYKI 52466 (CAS Number 102771-26-6), RO 246173 (CAS Number 159723-57-6), SCH 50911 (CAS Number 160415-07-6), Z 4105 (CAS Number 119737-52-9), RS 67333 (CAS Number 168986-60-5), NS 1546, ZM 241385 (CAS Number 139180-30-6), RO 249975 (i.e., [1S,3S(2′S),5R]-3-(1-Benzyl-5-oxopyrrolidin-2-ylmethyl)-5-(1H-imidazol-5-ylmethyl)cyclohexane-1-acetamide), AF 185 (i.e., 8-Methyl-3-(2-propynyl)-1,3,8-triazaspiro[4,5]decane-2,4-dione), CEP 427, CX 423, CX 438, CX 480, CDP-ethanolamine, GT 4003, GT 4011, GT 5011, MS 430 (CAS Number 122113-44-4), MBF 379 (i.e., [3,3-Bis(hydroxymethyl)-8-hydroxy-3,4-dihydro-2H-1,4-benzoxazin-5-yl][3′,5′-dihydroxy-4′-(2-oxo-2-phenylethoxy)phenyl]methanone), NGD 187 (CAS Number 163565-48-8), DUP 856, MR 3066, MF 8615 (i.e., 5-Amino-6-chloro-4-hydroxy-3,4-dihydro-1H-thiopyrano-[3,4-b]quinoline), himbacine, ABS 300, RJR 2403 (CAS Number 538-79-4), MF 268 (CAS Number 174721-00-7), RO 465934 (i.e., N,N-Dimethylcarbamic acid 3-(2-cyclohexylethyl)-2,3,3a,4,5,9b-hexahydro-1H-benz[e]indol-6-yl ester), NS 393, RGH 2716 (CAS Number 134069-68-4), WIN 678702 (12,12-Bis(3-furyl)-6,11-dihydro-6,11-ethanobenzo[b]quinolizinium chloride), RS 66252 (i.e., 1-Butyl-2-[(2′-(2H-tetrazol-5-yl)-biphenyl-4-yl)methyl]-1H-indole-3-carboxylic acid), AIT 034 (CAS Number 138117-48-3), NG 012 (CAS Number 131774-53-3), PD 142012 (CAS Number 5778-84-7), GT 4054, GT 4077, GT 4035, P 26 (CAS Number 152191-74-7), RGH 5279 (i.e., (−)-(13aR,13bS)-13a-Ethyl-2,3,5,6,13a,13b-hexahydro-1H-indolo[3,2,1-de]pyrido[3,2,1-ij][1,5]naphthyridine-12-carboxylic acid 2-acetoxyethyl ester), AIT 083, CeNeS, estradiol (i.e., 1,3,5(10)-Estratriene-3,17beta-diol), WAY 132983 ((3R,4R)-3-(3-hexylsulfanylpyrazin-2-yloxy)-1-azabicyclo[2.2.1]heptane hydrochloride), ABS 205, ABS 401, SX 3507 (i.e., 3-(3-Propyl-1,2,4-oxadiazol-5-yl)quinoxaline-2(1H)-one), ARR 17779 (i.e., (−)-Spiro[1-azabicyclo[2.2.2]octane-3,5-oxazolidine]-2-one), XE 991 (i.e., 10,10-bis(4-Pyridylmethyl)anthracen-10(9H)-one), phenethylnorcymserine, RO 657199, RJR 1781 (i.e., R(+)-2-(3-pyridyl)-1-azabicyclo[2.2.2.]octane), RJR 1782 (i.e., SH-2-(3-pyridyl)-1-azabicyclo[2.2.2doctane), gilatide, tolserine, TC 2559 (i.e., (E)-N-Methyl-443-(5-ethoxypyridin)yl]-3-buten-1-amine), ER 127528 (i.e., 1-(3-Fluorobenzyl)-4-[(2-fluoro-5,6-dimethoxy-1-indanone-2-yl)methyl]piperidine hydrochloride), thiatolserine, targacept, axonyx, cymserine, thiacymserine, monoclonal antibody 266, Apan-CH, DP 103, SPI 339 (i.e., 4-[3-(4-Oxo-4,5,6,7-tetrahydroindol-1-yl)propionylamino]benzoic acid ethyl ester), S 37245 (i.e., 4-(1,4-Benzodioxan-5-yl)-1-[3(S)-hydroxy-5-nitro-indan-2-yl]-piperazine), LLG 88, AZD 2858, trometamol, AN 240, NG 002 (i.e., 5-Hydroxy-5-(2-hydroxy-1-methylethyl)-4-methoxyfuran-2(5H)-one), UCB 29427 (i.e., 2-Cyclopropyl-4-(cyclopropylamino)-6-(morpholino)-1,3,5-triazine), TRH-SR, RO 401641 (CAS Number 122199-02-4), MPV 1743AIII (CAS Number 150586-64-4), IDRA 21 (CAS Number 22503-72-6), CEP 431, ACPD (CAS Number 67684-64-4), CT 3577 (i.e., 3,7-Dimethyl-1-[11-(3,4,5-trimethoxybenzylamino)-11-oxoundecyl]xanthine), CT 2583, and NXD 9062. The pharmaceutical compositions may comprise one or more pharmaceutically acceptable carriers.

The invention provides pharmaceutical combinations comprising a therapeutically effective amount of at least one cinnamide compound and one or more second-line active ingredients selected from the group consisting of cholinesterase inhibitors; AMPA receptor antagonists; and NMDA receptor antagonists.

The invention provides pharmaceutical combinations comprising a therapeutically effective amount of at least one cinnamide compound and one or more second-line active ingredients selected from the group consisting of cholinesterase inhibitors; AMPA receptor antagonists; NMDA receptor antagonists; pramiracetam; aniracetam; acetylcholine releasing stimulants; calcium channel agonists; free radical scavengers; platelet activating factor antagonists; platelet aggregation antagonists; insulin sensitizers; peroxisome proliferator-activated receptor agonists; peroxisome proliferator-activated receptor gamma agonists; monoamine oxidase B inhibitors; carnitine acetyltransferase stimulants; NSAIDs; nerve growth factor agonists; beta-amyloid inhibitors; immunomodulators; and NF-kappa B inhibitors.

The invention provides pharmaceutical combinations comprising a therapeutically effective amount of at least one cinnamide compound and one or more second-line active ingredients selected from the group consisting of cholinesterase inhibitors; AMPA receptor antagonists; NMDA receptor antagonists; pramiracetam; aniracetam; acetylcholine releasing stimulants; calcium channel agonists; free radical scavengers; platelet activating factor antagonists; platelet aggregation antagonists; insulin sensitizers; peroxisome proliferator-activated receptor agonists; peroxisome proliferator-activated receptor gamma agonists; monoamine oxidase B inhibitors; carnitine acetyltransferase stimulants; NSAIDs; nerve growth factor agonists; beta-amyloid inhibitors; immunomodulators; NF-kappa B inhibitors; thyrotropin releasing hormone agonists; dopamine D2 receptor antagonists; serotonin 2 receptor antagonists; muscarinic M1 receptor agonists; alpha 1 adrenoceptor agonists; serotonin 3 receptor antagonists; dopamine D2 receptor agonists; dopamine D2 receptor antagonists; serotonin 1A receptor agonists; serotonin 2A receptor antagonists; glucocorticoid antagonists; progesterone antagonists; HMG-CoA reductase inhibitors; adenosine uptake inhibitors; phosphodiesterase inhibitors; acetylcholine receptor agonists; membrane permeability enhancers; cannabinoid 1 receptor antagonists; cannabinoid receptor agonists; angiogenesis inhibitors; immunosuppressants; tubulin antagonists; thromboxane A2 synthase inhibitors; antioxidants; alpha adrenoreceptor antagonists; estrogen agonists; 3-beta hydroxysteroid dehydrogenase inhibitors; signal transduction pathway inhibitors; melatonin receptor agonists; immunostimulants; HIV entry inhibitors; sodium channel antagonists; microtubule inhibitors; glycine NMDA agonists; adenosine A1 receptor antagonists; ATPase stimulants; mitochondrial function enhancers; and growth hormone releasing factor agonists.

The invention provides pharmaceutical combinations comprising a therapeutically effective amount of at least one cinnamide compound and one or more second-line active ingredients selected from the group consisting of donepezil, 3-(2-cyanophenyl)-5-(2-pyridyl)-1-phenyl-1,2-dihydropyridin-2-one, and memantine.

The invention provides pharmaceutical combinations comprising a therapeutically effective amount of at least one cinnamide compound and one or more second-line active ingredients selected from the group consisting of donepezil, huperzine A, tacrine, rivastigmine, galantamine, pramiracetam, aniracetam, nefiracetam, EGb 761, rosiglitazone, rasagiline, levacecarnine, celecoxib, 3-(2-cyanophenyl)-5-(2-pyridyl)-1-phenyl-1,2-dihydropyridin-2-one, talampanel, becampanel, memantine, neramexane, xaliproden, tarenflurbil, tramiprosate, and leuprorelin-D.

The invention provides pharmaceutical combinations comprising a therapeutically effective amount of at least one cinnamide compound and one or more second-line active ingredients selected from the group consisting of donepezil, huperzine A, tacrine, rivastigmine, galantamine, pramiracetam, aniracetam, nefiracetam, EGb 761, rosiglitazone, rasagiline, levacecarnine, celecoxib, 3-(2-cyanophenyl)-5-(2-pyridyl)-1-phenyl-1,2-dihydropyridin-2-one, talampanel, becampanel, memantine, xaliproden, tarenflurbil, tramiprosate, leuprorelin-D, taltirelin, risperidone, cevimeline, modafinil, alosetron, aripiprazole, mifepristone, atorvastatin, propentofylline, choline alfoscerate, FPF 1070 (CAS Number 443637-01-8), rimonabant, dronabinol, docosahexaenoic acid, paclitaxel, triflusal, idebenone, nicergoline, conjugated estrogens, trilostane, simvastatin, selegiline, ramelteon, immune globulin, icosapentethyl ester, procaine, CPH 82, cycloserine, KW 3902 (CAS Number 136199-02-5), triacetyluridine, estrogen dementia therapeutics (e.g., MIGENIX, Vancouver, Canada), and tesamorelin. In other embodiments, the second-line active ingredients can be one or more compounds selected from the group consisting of levprorelin, prasterone, peptide T (CAS Number 53-43-0), besipiridine, lexipafant, stacofylline, SGS 742 (CAS Number 123690-78-8), T 588 (CAS Number 142935-03-3), nerispiridine; dexanabinol, sabcomeline, GTS 21 (CAS Number 156223-05-1), CX 516 (CAS Number 154235-83-3), ABT 089 (CAS Number 161417-03-4), anapsos, tesofensine, SIB 1553A (i.e., 4-[[2-(1-methyl-yl-2-pyrrolidinyl)ethyl]thia]phenol), ladostigil, radequinil, GPI 1485, ispronicline; arundic acid, MEM 1003 (i.e., 3-Isopropyl 5-(2-methoxyethyl) 4-(2-chloro-3-cyanophenyl)-2,6-dimethylpyridine-3,5-dicarboxylate), V 3381 (i.e., 2-(2,3-Dihydro-1H-inden-3-ylamino)acetamide hydrochloride), farampator, paliroden, prasterone-paladin, urocortin, DP b99 (i.e., 2,2′-(Ethylenedioxy)bis(2-(1-phenylene)bis[N-[2-[2-(octyloxy)ethoxy]-2-oxoethyl]imino]bis(acetic acid)), capserod, DU 125530, bapineuzumab, AL 108 (i.e., L-Asparaginyl-L-alanyl-L-prolyl-L-valyl-L-seryl-L-isoleucyl-L-prolyl-L-glutamine), DAS 431, DEBIO 9902, DAR 1-00; mitoquinone, IPL 455903 (i.e., 5(S)-[3-(Cyclopentyloxy)-4-methoxyphenyl]-3(S)-(3-methylbenzyl)piperidin-2-one), E2CDS, PYM 50028, PBT 2, lecozotan, SB 742457, CX 717, AVE 1625 (i.e., 1-(bis(4-chlorophenyl)methyl)-3-((3,5-difluorophenyl)(methylsulfonyl)methylene)azetidine), LY 450139 (i.e., N2-[2(s)-Hydroxy-3-methylbutyryl]-N1-[3-methyl-2-oxo-2,3,4,5-tetrahydro-1H-3-benzazepin-1(S)-yl]-L-alaninamide), EM 1421 (i.e., 4,4′-[(2R,3S)-2,3-Dimethylbutane-1,4-diyl]bis(1,2-dimethoxybenzene), SRN 001, TTP 488, PRX 03140, dimebolin, glycine-proline-glutamate, C105, AL 208, MEM 3454, AC 1202, L 830982, LY 451395 (i.e., (R)—N-[2-[4′-(methylsulfonamidomethyl)biphenyl-4-yl]propyl]propane-2-sulfonamide), MK 0249, LY 2062430, diethylnorspermine, neboglamine, S18986, SA 4503 (CAS Number 165377-44-6), GRI 1, S17092 (i.e., (2S,3aS,7aS)-1{[(R,R)-2-Phenylcyclopropyl]carbonyl}-2-[(thiazolidin-3-yl)carbonyl]octahydro-1H-indole), SL 251188, EUK 189, R 1450, 6,6-dimethyl-3-(2-hydroxyethyl)thio-1-(thiazol-2-yl)-6,7-dihydro-2-benzothiophen-4(5H)-one, CERE 110, dexefaroxan, CAD 106, HF 0220, HF 0420, EHT 0202, VP 025, MEM 1414, BGC 201259 (i.e., N,N-Dimethylcarbamic acid, 4-[1(S)-(methylamino)-3-(4-nitrophenoxy)propyl]phenyl ester), EN 100, ABT 834, ABT 239 (i.e., 4-[2-[2-[(2R)-2-Methylpyrrolidinyl]ethyl]-benzofuran-5-yl]benzonitrile), SGS 518, R 1500, C 9138, SSR 180711, alfatradiol, R 1577, T 817MA (i.e., 1-[3-[2-(1-Benzothien-5-yl)ethoxy]propyl]azetidin-3-olmaleate), CNP 1061 (i.e., 4-Methyl-5-(2-nitrooxyethyl)thiazole), KTX 0101 (i.e., sodium beta-hydroxybutyrate), GSK 189254 (i.e., 6-[3-Cyclobutyl-2,3,4,5-tetrahydro-1H-benzo[d]azepin-7-yloxy]-N-methylnicotinamide), AZD 1080, ACC 001, PRX 07034, midazolam, R-phenserine, AZD 103 (CAS Number 488-59-5), SN 522, NGX 267 (CAS Number 503431-81-0), N-PEP-12, RN 1219, FGLL, AVE 8112, EVT 101, NP 031112, MK 0752, MK 0952, LX 6171, PAZ 417, AV 965, PF 3084014, SYN 114, GSI 953, SAM 315, SAM 531, D-serine, leteprinim potassium, BR 16A (CAS Number 149175-77-9), RPR 107393 (CAS Number 190841-57-7), NXD 2858, REN 1654, CDD 0102, NC 1900 (CAS Number 132925-74-7), ciclosporin, NCX 2216 (i.e., (E)-4-(Nitrooxy)butyl 3-[4-[2-(2-fluorobiphenyl-4-yl)propanoyloxy]-3-methoxyphenyl]acrylate), NXD 3109, NXD 1191, ZSET 845 (i.e., 3,3-diphenylimidazo[1,2-a]pyridin-2-(3H)-one), ET 002, NT 13, RO 638695 (i.e., [1,6-(1,6-dioxohexyl)]dipyrrolidine-(2R)-carboxylic acid), bisnorcymserine, BA 1016, XD 4241, EUK 207 (i.e., (SP-5-13)-(acetato-κO)[13,16,19,22-tetraoxa-3,6-diazatricyclo[21.3.18.12]octacosa-1(27),2,6,8,10,12(28),23,25-octaene-27,28-diolato(2-)-κN3,κN6,κO27,κO28]manganese), LG 617 inhibitors, ZSET 1446, PAN 811, F 14413 (i.e., 2-[5-Fluoro-2(S)-methoxy-2,3-dihydro-1,4-benzodioxin-2-yl]-4,5-dihydro-1H-imidazole), FP 7832 (i.e., N-[2-(5-Methoxy-1-nitroso-1H-indol-3-yl)ethyl]acetamide), ARA 014418 (i.e., N-(4-methoxybenzyl)-N′-(5-nitro-1,3-thiazol-2-yl)urea), AZD 3102, KP 544 (i.e., 2-amino-5-(4-chlorophenylethynyl)-4-(4-trans-hydroxycyclohexylamino)pyrimidine), DP 155, 5-Chloro-N-[3-[2-(dimethylamino)ethyl]-1H-indol-5-yl]naphthalene-2-sulfonamide, TAK 070, huperzine, N-[2-(3,5-Dimethyladamant-1-yl)ethyl]acetamidine hydrochloride, 6-[4-[(Dimethylamino)methyl]-5-ethyl-2-methoxyphenyl]pyridin-2-amine, 4,6-diphenyl-3-(4-(pyrimidin-2-yl)piperazin-1-yl)pyridazine, N-[(1S,2R)-3-(3,5-Difluorophenyl)-1-hydroxy-1-[(5S,6R)-5-methyl-6-(neopentyloxy)morpholin-3-yl]propan-2-yl]acetamide hydrochloride, N-[(1R,2S)-3-(3,5-Difluorophenyl)-1-hydroxy-1-[(2R,4R)-4-phenoxypyrrolidin-2-yl]propan-2-yl]-3-[(R)-2-(methoxymethyl)pyrrolidine-1-carbonyl]-5-methylbenzamide, R 1589, midafotel, phenserine, coluracetam, physostigmine, cipralisant, nitroflurbiprofen, PPI 1019 (i.e., (3alpha,5beta,7alpha,12alpha)-trihydroxycholan-24-oyl-L-leucyl-L-valyl-L-phenylalanyl-L-phenylalanyl-L-alanine), dapsone, MDL 100453 (CAS Number 129938-34-7), NS 377, midaxifylline, propofol phosphate, metrifonate, ceronapril, tenilsetam, sufoxazine, seglitide, ebiratide, nebracetam, milacemide, iododoxorubicin, SM 10888 (CAS Number 129297-21-8), U 80816 (CAS Number 138554-11-7), YM 954 (CAS Number 132041-85-1), SUT 8701 (CAS Number 123577-73-1), apovincamine, FR 121196 (CAS Number 133920-65-7), LY 274614 (CAS Number 136109-04-1), CL 275838 (CAS Number 115931-65-2), igmesine, K 7259 (CAS Number 133667-88-6), vinconate, itasetron, CL 287663 (CAS Number 125109-98-0), WAY 100289 (CAS Number 136013-69-9), SR 46559A (CAS Number 137733-33-6), GYKI 46903 (CAS Number 142999-59-5), L 670548 (CAS Number 121564-89-4), Y 29794 (CAS Number 129184-48-1), AF 125 (CAS Number 7631-86-9), KFM 19 (CAS Number 133058-72-7), ST 796 (i.e., (S)-3-[3-(trifluoromethyl)benzoyl)amino]hexahydroazepin-2-one), RU 33965 (CAS Number 122321-05-5), SDZ 210086 (i.e., (−)-1′,2(S)-Dimethylspiro[1,3-dioxolan-4,4′-piperidine]), L 689660 (CAS Number 144860-79-7), L 689560 (CAS Number 139051-78-8), ST 618 (i.e., 1-(6,7-Dimethoxy-1,2,3,4-tetrahydro-2-naphthyl)-4-hydroxy pyrrolidin-2-one), U 74500A (CAS Number 110101-65-0), GEA 857 (CAS Number 120493-42-7), BIBN 99 (CAS Number 145301-48-0), DX 9366, ONO 1603 (CAS Number 114668-76-7), MDL 102234 (CAS Number 137766-81-5), P 9939 (CAS Number 157971-37-4), PD 140532 (CAS Number 157971-39-6), azetirelin, MR 16728 (CAS Number 147614-21-9), dabelotine, MDL 102503 (i.e., 8-[1(R)-methyl-2-phenylethyl]-1,3-dipropyl-7H-xanthine), PD 141606 (i.e., (±)-(Z)-3-(3-Phenyl-2-propynyloxyimino)-1-azabicyclo[2.2.1]heptane), SNK 882 (CAS Number 152221-12-0), L 696986 (CAS Number 141553-45-9), tazomeline, LY 235959 (CAS Number 137433-06-8), 2-(2-thiooxopyrrolidin-1-yl)acetamide, AK 30 NGF, ABT 418 (CAS Number 147402-53-7), itameline, HUP 13, sibopirdine, KST 5452 (CAS Number 157998-88-4), TJ 54, U 92798 (i.e., 7-[4-[Bis(4-fluorophenyl)methyl]perhydro-1,4-diazepin-1-ylmethyl]-4-isopropyl-2-methoxy-2,4,6-cycloheptatrien-1-one), U 92032 (CAS Number 142223-92-5), 3-(sulfamoyloxy)estra-1,3,5(10)-trien-17-one, P 11012 (CAS Number 164723-36-8), A 82695 (CAS Number 147388-86-1), FR 76659 (CAS Number 116904-25-7), apaxifylline, CX 417, 7 MEOTA (CAS Number 5778-80-3), BU 4514N(CAS Number 151013-39-7), pregnenolone, mexidol, ST 857 (CAS Number 154755-63-2), RU 49041 (CAS Number 123828-80-8), RU 35929 (CAS Number 111711-47-8), P 878184, P 128 (CAS Number 157716-52-4), eurystatin A, eurystatin B, LK 12, NBI 108, NBI 107, NBI 117, L 705106, bacoside A+B, clausenamide, SM 21 (CAS Number 155156-22-2), alaptide, RS 17017 (i.e., 1-(4-Amino-5-chloro-2-methoxyphenyl)-5-(1-piperidinyl)-1-pentanone hydrochloride), AF 150(S) (i.e., (S)-[1-Methyl-piperidine-4-spiro-(2′-methylthiazoline)]), RO 153505 (CAS Number 78771-13-8), PV 113 (i.e., 1,2,3,4-Tetrahydropyrrolo-[1,2-a]-pyrazine), arisugacin, A 98284 (i.e., 2(R)-(3-Methylisoxazol-5-yl) quinuclidine), AP 5 (CAS Number 136941-85-0), BD 1054, SDZ NDD 094 (i.e., bis-(2-(2-methylimidazol-1-yl]methyl)-pyridine-tris(hydrogen-fumarate), AZ 36041 (CAS Number 173324-76-0), quilostigmine, A 84543 (i.e., 3-[1-Methylpyrrolidin-2-(S)-ylmethoxy]pyridine fumarate), BTG 4247 (i.e., (2-[2-Chloroethoxy[4-(dimethylamino)phenyl]phosphoryl]-acetohydrazide), CGP 50068 (CAS Number 158647-49-5), cerebrocrast, desferri-nordanoxamine, isolichenan, MHP 133 (i.e., 3-(N,N-dimethylcarbamoyloxy)-1-methyl-2-(4-phenyl-semicarbazonomethyl)pyridinium chloride), FR 152558 (CAS Number 151098-08-7), GVS 111 (CAS Number 157115-85-0), P 11149 (CAS Number 164724-79-2), PDC 008004, KST 2818 (CAS Number 158623-26-8), KST 5410 (CAS Number 158623-27-9), RU 52583 (CAS Number 123829-33-4), PD 151832 (CAS Number 149929-39-5), UCL 1199 (i.e., 442-[(5-nitropyridin-2-ylsulfanyl)ethyl]-1H-imidazole), isovanihuperzine A, SIB 1765F (CAS Number 179120-52-6), JWS USC 751X (i.e., 3-[[[2-[[(5-dimethylaminomethyl)-2-furanyl]methyl]thio]ethyl]amino]-4-nitropyridazine), GR 175737 (i.e., 3-(4-Chlorobenzyl)-5-[2-(1H-imidazol-4-yl)ethyl]-1,2,4-oxadiazole), KS 505A (CAS Number 131774-53-3), ZTTA 1 (i.e., N-benzyloxycarbonyl-thioprolyl-thioprolynal-dimethylaceta 1), AGN 190837 (CAS Number 136527-40-7), P 10358 (188240-59-7), WAY 131256 (CAS Number 174001-71-9), DBO 83 (i.e., 3-(6-chloropyridazin-3-yl)-diazabicyclo[3.2.1]octane dihydrochloride monohydrate), FUB 181 (CAS Number 152029-80-6), RJR 2557, WSU 2088, LVV-haemorphin-7, M 40 (i.e., galanin[1-12]-Pro3-(Ala-Leu)-2-Ala-NH₂), SIB 1757, SKF 74652 (i.e., [5-chloro-2-(4-methoxy phenyl)-3-benzofuranyl][4-[3-(diethylamino)-propoxy]phenyl]methanone), CGP 71982, SCH 57790 (i.e., 4-cyclohexyl-alpha-[4-[[4-methoxyphenyl]sulfinyl]phenyl]-1-piperazineacetonitrile), Putrescine-D-YiAbeta11, DU 14 (i.e., p-O-(sulfamoyl)-N-tetradecanoyl tyramine), CLZ 4, SL 340026, PPRT 424, ciproxifan, UR 1827 (i.e., 2-(1-benzylpiperidin-4-yl)-1-[4-(5-methylpyrimidin-4-ylamino)phenyl]-1-ethanone), caproctamine, TGS 20 (i.e., L-pyroglutamil-D-alanine amide), PG 9 (i.e., alpha-tropanyl 2-[(4-bromo)phenyl]propionate), TEI 3356 (i.e., (16S)-15-Deoxy-16-hydroxy-16-methyl-9-(O)-methano-DELTA6(9alpha)-prostaglandin I1), LY 392098 (i.e., Thiophene, 3-[(2-methylethyl-2)sulphonylaminopropyl-2]phenyl-4-yl-), PG 1000, DM 232, NEPP 11 (i.e., 12-iso-15-Deoxy-18-(4-methyl)phenyl-13,14-dihydro-delta7-prostaglandinA1 methyl ester), VA 100 (i.e., (2,3-Dihydro-2-[[(4-fluorobenzoyl)amino]ethyl]-1-methyl-5-phenyl-1H-1,4-benzodiazepine), VA 101 (i.e., (2,3-dihydro-2-[[(2-thienylcarbonyl)amino]ethyl]-1-methyl-5-phenyl-1H-1,4-benzodiazepine), NC 111585 (i.e., (3S)-1,3-Bis-[3-[(3-azabicylo[2.2.2]octanyl)-1,2,5-thiadiazol-4-yloxy]-1-propyn-1-yl]benzene, 2L-(+)-tartate), IN 201, imoproxifan, kanokodiol, picroside I, picroside II, DM 235 (i.e., 1-(4-Benzoylpiperazin-1-yl)propan-1-one), monoclonal antibody 10D5, JLK2, JLK 6, JLK 7, DAPT (i.e., N-[N-(3,5-difluorophenacetyl)-L-alanyl]-S-phenylglycine t-butyl ester), huperine X, SGS 111 (i.e., (S)-ethyl 2-[1-(2-phenylacetyl)pyrrolidine-2-carboxamido]acetate), NP 7557, C 9136, C 7617, R 1485, rofecoxib, velnacrine, montirelin, lazabemide, ORG 2766 (CAS Number 50913-82-1), sabeluzole, adafenoxate, CAS Number 9061-61-4, ipidacrine, bemesetron, idazoxan, linopirdine, selfotel, suritozole, milameline, xanomeline, TJ 960, fasoracetam, eptastigmine, ensaculin, zanapezil, posatirelin, zacopride, RS 86 (CAS Number 3576-73-6), ORG 5667 (CAS Number 37552-33-3), RX 77368 (CAS Number 76820-40-1), BMS 181168 (CAS Number 123259-91-6), BY 1949 (CAS Number 90158-59-1), AWD 5239 (CAS Number 109002-93-9), YM 796 (171252-79-2), aloracetam, CI-933 (CAS Number 91829-95-7), ST 793 (CAS Number 99306-37-3), cebaracetam, zifrosilone, talsaclidine, alvameline, JTP 2942 (148152-77-6), OPC 14117 (CAS Number 103233-65-4), elziverine, AP 521 (i.e., N-(1,3-Benzodioxol-5-ylmethyl)-1,2,3,4-tetrahydro[1]benzothieno[2,3-c]pyridine-3(R)-carboxamide hydrochloride), S 8510 (CAS Number 151466-23-8), JTP 4819 (CAS Number 162203-65-8), icopezil, SC 110, FK 960 (CAS Number 133920-70-4), DMP 543 (CAS Number 160588-45-4), ganstigmine, CI 1017 (i.e., (R)-(−)-(Z)-1-Azabicyclo[2.2.1]heptan-3-one, O-(3-(3′-methoxyphenyl)-2-propynyl)-oxime maleate), T 82 (i.e., 2-[2-(1-Benzylpiperidin-4-yl)ethyl]-2,3-dihydro-9-methoxy-1H-pyrrolo[3,4-b]quinolin-1-one hemifumarate), NGD 971, vaccine of Aspartyl-alanyl-glutamyl-phenylalanyl-arginyl-histidyl-aspartyl-seryl-glycyl-tyrosyl-glutamyl-valyl-histidyl-histidyl-glutaminyl-lysyl-leucyl-valyl-phenylalanyl-phenylalanyl-alanyl-glutamyl-aspartyl-valyl-glycyl-seryl-asparaginyl-lysyl-glycyl-alanyl-isoleucyl-isoleucyl-glycyl-leucyl-methionyl-valyl-glycyl-glycyl-valyl-valyl-isoleucyl-alanine, PBT 1 (CAS Number 130-26-7), TCH 346, FK 962 (i.e., N-(1-acetylpiperidin-4-yl)-4-fluorobenzamide), voxergolide, KW 6055 (CAS Number 63233-46-5), thiopilocarpine, ZK 93426 (CAS Number 89592-45-0), SDZ NVI 085 (CAS Number 104195-17-7), CI 1002 (CAS Number 149028-28-4), Z 321 (CAS Number 130849-58-0), mirisetron, CHF 2060 (i.e., N-Heptylcarbamic acid 2,4a,9-trimethyl-2,3,4,4a,9,9a-hexahydro-1,2-oxazino[6,5-b]indol-6-yl ester-L-tartrate), gedocarnil, terbequinil, HOE 065 (CAS Number 123060-44-6), SL 650102, GR 253035, ALE 26015, SB 271046 (i.e., 5-Chloro-N-(4-methoxy-3-piperazin-1-yl-phenyl)-3-methyl-2-benzothiophenesulfonamide), iAbeta5, SCH 211803 (i.e., Piperidine, 1-[1-(3-methyl-2-aminophenyl)carbonylpiperidin-4-yl]-4-[(3-chlorophenyl)sulphonylphenyl-4]methyl-), EVT 301, alpha-Linolenic acid/linoleic acid, Kamikihi-To, siagoside, FG 7142 (CAS Number 78538-74-6), RU 47067 (CAS Number 111711-92-3), RU 35963 (CAS Number 139886-03-6), FG 7080 (CAS Number 100332-18-1), E 2030 (CAS Number 142007-70-3), transforming growth factor beta-1, A 72055 (i.e., 2′,1-Dimethylspiro[piperidine-4,5′ oxazolidine]-3′-carboxaldehyde), NS 626, dimiracetam, GT 3001, GT 2501, GT 2342, GT 2016 (CAS Number 152241-24-2), ORG 20091 (CAS Number 141545-50-8), BCE 001 (CAS Number 95678-81-2), CGP 35348 (CAS Number 123690-79-9), WAY 100635 (CAS Number 146714-97-8), E 4804 (CAS Number 162559-34-4), LIGA 20 (CAS Number 126586-85-4), NG 121 (i.e., 2-[4,8-Dimethyl-3(E),7(E)-nonadienyl]-3,5-dihydroxy-2-methyl-3,4,7,9-tetrahydro-2H-furo[3,4-h]-1-benzopyran-7-one), MF 247 (i.e., N-[10-(Diethylamino)decyl]carbamic acid (3aS,8aR)-1,3a,8-trimethyl-1,2,3,3a,8,8a-hexahydropyrrolo[2,3-b]indol-5-yl ester), JTP 3399 (i.e., N-Benzyl-2(S)-[2(S)-(phenoxyacetyl)pyrrolidin-1-ylcarbonyl]pyrrolidine-1-carboxamide), KF 17329, thioperamide, F 3796 (i.e., 1-[2-(1-Benzylpiperidin-4-yl)ethyl]-3-[3,4-(methylene-dioxy)benzoyl]thiourea), GT 4001, GT 4002, FPL 14995 (CAS Number 123319-03-9), RU 3433.2 (CAS Number 137157-58-5), SR 96777A (CAS Number 115767-94-7), SIB T1980, NS 649 (CAS Number 146828-02-6), PD 142505 (CAS Number 149929-08-8), GYKI 52466 (CAS Number 102771-26-6), RO 246173 (CAS Number 159723-57-6), SCH 50911 (CAS Number 160415-07-6), Z 4105 (CAS Number 119737-52-9), RS 67333 (CAS Number 168986-60-5), NS 1546, ZM 241385 (CAS Number 139180-30-6), RO 249975 (i.e., [1S,3S(2′S),5R]-3-(1-Benzyl-5-oxopyrrolidin-2-ylmethyl)-5-(1H-imidazol-5-ylmethyl)cyclohexane-1-acetamide), AF 185 (i.e., 8-Methyl-3-(2-propynyl)-1,3,8-triazaspiro[4,5]decane-2,4-dione), CEP 427, CX 423, CX 438, CX 480, CDP-ethanolamine, GT 4003, GT 4011, GT 5011, MS 430 (CAS Number 122113-44-4), MBF 379 (i.e., [3,3-Bis(hydroxymethyl)-8-hydroxy-3,4-dihydro-2H-1,4-benzoxazin-5-yl][3′,5′-dihydroxy-4′-(2-oxo-2-phenylethoxy)phenyl]methanone), NGD 187 (CAS Number 163565-48-8), DUP 856, MR 3066, MF 8615 (i.e., 5-Amino-6-chloro-4-hydroxy-3,4-dihydro-1H-thiopyrano-[3,4-b]quinoline), himbacine, ABS 300, RJR 2403 (CAS Number 538-79-4), MF 268 (CAS Number 174721-00-7), RO 465934 (i.e., N,N-Dimethylcarbamic acid 3-(2-cyclohexylethyl)-2,3,3a,4,5,9b-hexahydro-1H-benz[e]indol-6-yl ester), NS 393, RGH 2716 (CAS Number 134069-68-4), WIN 678702 (12,12-Bis(3-furyl)-6,11-dihydro-6,11-ethanobenzo[b]quinolizinium chloride), RS 66252 (i.e., 1-Butyl-2-[(2′-(2H-tetrazol-5-yl)-biphenyl-4-yl)methyl]-1H-indole-3-carboxylic acid), AIT 034 (CAS Number 138117-48-3), NG 012 (CAS Number 131774-53-3), PD 142012 (CAS Number 5778-84-7), GT 4054, GT 4077, GT 4035, P 26 (CAS Number 152191-74-7), RGH 5279 (i.e., (−)-(13aR,13bS)-13a-Ethyl-2,3,5,6,13a,13b-hexahydro-1H-indolo[3,2,1-de]pyrido[3,2,1-ij][1,5]naphthyridine-12-carboxylic acid 2-acetoxyethyl ester), AIT 083, CeNeS, estradiol (i.e., 1,3,5(10)-Estratriene-3,17beta-diol), WAY 132983 ((3R,4R)-3-(3-hexylsulfanylpyrazin-2-yloxy)-1-azabicyclo[2.2.1]heptane hydrochloride), ABS 205, ABS 401, SX 3507 (i.e., 3-(3-Propyl-1,2,4-oxadiazol-5-yl)quinoxaline-2(1H)-one), ARR 17779 (i.e., (−)-Spiro[1-azabicyclo[2.2.2]octane-3,5-oxazolidine]-2-one), XE 991 (i.e., 10,10-bis(4-Pyridylmethyl)anthracen-10(9H)-one), phenethylnorcymserine, RO 657199, RJR 1781 (i.e., R(+)-2-(3-pyridyl)-1-azabicyclo[2.2.2]octane), RJR 1782 (i.e., S(−)-2-(3-pyridyl)-1-azabicyclo[2.2.2.]octane), gilatide, tolserine, TC 2559 (i.e., (E)-N-Methyl-4-[3-(5-ethoxypyridin)yl]-3-buten-1-amine), ER 127528 (i.e., 1-(3-Fluorobenzyl)-4-[(2-fluoro-5,6-dimethoxy-1-indanone-2-yl)methyl]piperidine hydrochloride), thiatolserine, targacept, axonyx, cymserine, thiacymserine, monoclonal antibody 266, Apan-CH, DP 103, SPI 339 (i.e., 4-[3-(4-Oxo-4,5,6,7-tetrahydroindol-1-yl)propionylamino]benzoic acid ethyl ester), S 37245 (i.e., 4-(1,4-Benzodioxan-5-yl)-1-[3(S)-hydroxy-5-nitro-indan-2-yl]-piperazine), LLG 88, AZD 2858, trometamol, AN 240, NG 0.002 (i.e., 5-Hydroxy-5-(2-hydroxy-1-methylethyl)-4-methoxyfuran-2(5H)-one), UCB 29427 (i.e., 2-Cyclopropyl-4-(cyclopropylamino)-6-(morpholino)-1,3,5-triazine), TRH-SR, RO 401641 (CAS Number 122199-02-4), MPV 1743AIII (CAS Number 150586-64-4), IDRA 21 (CAS Number 22503-72-6), CEP 431, ACPD (CAS Number 67684-64-4), CT 3577 (i.e., 3,7-Dimethyl-1-[11-(3,4,5-trimethoxybenzylamino)-11-oxoundecyl]xanthine), CT 2583, and NXD 9062.

In other embodiments, the invention provides kits comprising a therapeutically effective amount of: at least one cinnamide compound and one or more second-line active ingredients selected from the group consisting of cholinesterase inhibitors; AMPA receptor antagonists; and NMDA receptor antagonists.

The invention provides pharmaceutical kits comprising a therapeutically effective amount of at least one cinnamide compound and one or more second-line active ingredients selected from the group consisting of cholinesterase inhibitors; AMPA receptor antagonists; NMDA receptor antagonists; pramiracetam; aniracetam; acetylcholine releasing stimulants; calcium channel agonists; free radical scavengers; platelet activating factor antagonists; platelet aggregation antagonists; insulin sensitizers; peroxisome proliferator-activated receptor agonists; peroxisome proliferator-activated receptor gamma agonists; monoamine oxidase B inhibitors; carnitine acetyltransferase stimulants; NSAIDs; nerve growth factor agonists; beta-amyloid inhibitors; immunomodulators; and NF-kappa B inhibitors.

The invention provides pharmaceutical kits comprising a therapeutically effective amount of at least one cinnamide compound and one or more second-line active ingredients selected from the group consisting of cholinesterase inhibitors; AMPA receptor antagonists; NMDA receptor antagonists; pramiracetam; aniracetam; acetylcholine releasing stimulants; calcium channel agonists; free radical scavengers; platelet activating factor antagonists; platelet aggregation antagonists; insulin sensitizers; peroxisome proliferator-activated receptor agonists; peroxisome proliferator-activated receptor gamma agonists; monoamine oxidase B inhibitors; carnitine acetyltransferase stimulants; NSAIDs; nerve growth factor agonists; beta-amyloid inhibitors; immunomodulators; NF-kappa B inhibitors; thyrotropin releasing hormone agonists; dopamine D2 receptor antagonists; serotonin 2 receptor antagonists; muscarinic M1 receptor agonists; alpha 1 adrenoceptor agonists; serotonin 3 receptor antagonists; dopamine D2 receptor agonists; dopamine D2 receptor antagonists; serotonin 1A receptor agonists; serotonin 2A receptor antagonists; glucocorticoid antagonists; progesterone antagonists; HMG-CoA reductase inhibitors; adenosine uptake inhibitors; phosphodiesterase inhibitors; acetylcholine receptor agonists; membrane permeability enhancers; cannabinoid 1 receptor antagonists; cannabinoid receptor agonists; angiogenesis inhibitors; immunosuppressants; tubulin antagonists; thromboxane A2 synthase inhibitors; antioxidants; alpha adrenoreceptor antagonists; estrogen agonists; 3-beta hydroxysteroid dehydrogenase inhibitors; signal transduction pathway inhibitors; melatonin receptor agonists; immunostimulants; HIV entry inhibitors; sodium channel antagonists; microtubule inhibitors; glycine NMDA agonists; adenosine A1 receptor antagonists; ATPase stimulants; mitochondrial function enhancers; and growth hormone releasing factor agonists.

The invention provides pharmaceutical kits comprising a therapeutically effective amount of at least one cinnamide compound and one or more second-line active ingredients selected from the group consisting of donepezil, 3-(2-cyanophenyl)-5-(2-pyridyl)-1-phenyl-1,2-dihydropyridin-2-one, and memantine.

The invention provides pharmaceutical kits comprising a therapeutically effective amount of at least one cinnamide compound and one or more second-line active ingredients selected from the group consisting of donepezil, huperzine A, tacrine, rivastigmine, galantamine, pramiracetam, aniracetam, nefiracetam, EGb 761, rosiglitazone, rasagiline, levacecarnine, celecoxib, 3-(2-cyanophenyl)-5-(2-pyridyl)-1-phenyl-1,2-dihydropyridin-2-one, talampanel, becampanel, memantine, neramexane, xaliproden, tarenflurbil, tramiprosate, and leuprorelin-D.

The invention provides pharmaceutical kits comprising a therapeutically effective amount of at least one cinnamide compound and one or more second-line active ingredients selected from the group consisting of donepezil, huperzine A, tacrine, rivastigmine, galantamine, pramiracetam, aniracetam, nefiracetam, EGb 761, rosiglitazone, rasagiline, levacecarnine, celecoxib, 3-(2-cyanophenyl)-5-(2-pyridyl)-1-phenyl-1,2-dihydropyridin-2-one, talampanel, becampanel, memantine, xaliproden, tarenflurbil, tramiprosate, leuprorelin-D, taltirelin, risperidone, cevimeline, modafinil, alosetron, aripiprazole, mifepristone, atorvastatin, propentofylline, choline alfoscerate, FPF 1070 (CAS Number 143637-01-8), rimonabant, dronabinol, docosahexaenoic acid, paclitaxel, triflusal, idebenone, nicergoline, conjugated estrogens, trilostane, simvastatin, selegiline, ramelteon, immune globulin, icosapentethyl ester, procaine, CPH 82, cycloserine, KW 3902 (CAS Number 136199-02-5), triacetyluridine, estrogen dementia therapeutics (e.g., MIGENIX, Vancouver, Canada), and tesamorelin. In other embodiments, the second-line active ingredients can be one or more compounds selected from the group consisting of leuprorelin, prasterone, peptide T (CAS Number 53-43-0), besipiridine, lexipafant, stacofylline, SGS 742 (CAS Number 123690-78-8), T 588 (CAS Number 142935-03-3), nerispiridine, dexanabinol, sabcomeline, GTS 21 (CAS Number 156223-05-1), CX 516 (CAS Number 154235-83-3), ABT 089 (CAS Number 161417-03-4), anapsos, tesofensine, SIB 1553A (i.e., 4-[[2-(1-methyl-yl-2-pyrrolidinyl)ethyl]thia]phenol), ladostigil, radequinil, GPI 1485, ispronicline, arundic acid, MEM 1003 (i.e., 3-Isopropyl 5-(2-methoxyethyl) 4-(2-chloro-3-cyanophenyl)-2,6-dimethylpyridine-3,5-dicarboxylate), V 3381 (i.e., 2-(2,3-Dihydro-1H-inden-3-ylamino)acetamide hydrochloride), farampator, paliroden, prasterone-paladin, urocortin, DP b99 (i.e., 2,2′-(ethylenedioxy)bis(2,1-phenylene)bis[N-[2-[2-(octyloxy)ethoxy]-2-oxoethyl]imino]bis(acetic acid)), capserod, DU 125530, bapineuzumab, AL 108 (i.e., L-Asparaginyl-L-alanyl-L-prolyl-L-valyl-L-seryl-L-isoleucyl-L-prolyl-L-glutamine), DAS 431, DEBIO 9902, DAR 100, mitoquinone, IPL 455903 (i.e., 5(S)-[3-(Cyclopentyloxy)-4-methoxyphenyl]-3(S)-(3-methylbenzyl)piperidin-2-one), E2CDS, PYM 50028, PBT 2, lecozotan; SB 742457, CX 717, AVE 1625 (i.e., 1-(bis(4-chlorophenyl)methyl)-3-((3,5-difluorophenyl)(methylsulfonyl)methylene)azetidine), LY 450139 (i.e., N2-[2(s)-Hydroxy-3-methylbutyryl]-N1-[3-methyl-2-oxo-2,3,4,5-tetrahydro-1H-3-benzazepin-1(S)-yl]-L-alaninamide), EM 1421 (i.e., 4,4′-[(2R,3S)-2,3-Dimethylbutane-1,4-diyl]bis(1,2-dimethoxybenzene), SRN 001, TTP 488, PRX 03140, dimebolin, glycine-proline-glutamate, C105, AL 208, MEM 3454, AC 1202, L 830982, LY 451395 (i.e., (R)—N-[2-[4′-(methylsulfonamidomethyl)biphenyl-4-yl]propyl]propane-2-sulfonamide), MK 0249, LY 2062430, diethylnorspermine, neboglamine, S18986, SA 4503 (CAS Number 165377-44-6), GRI 1, S 17092 (i.e., (2S,3aS,7aS)-1{[(R,R)-2-phenylcyclopropyl]carbonyl}-2-[(thiazolidin-3-yl)carbonyl]octahydro-1H-indole), SL 251188, EUK 189, R 1450, 6,6-dimethyl-3-(2-hydroxyethyl)thio-1-(thiazol-2-yl)-6,7-dihydro-2-benzothiophen-4(5H)-one, CERE 110, dexefaroxan, CAD 106, HF 0220, HF 0420, EHT 0202, VP 025, MEM 1414, BGC 201259 (i.e., N,N-dimethylcarbamic acid, 4-[1(S)-(methylamino)-3-(4-nitrophenoxy)propyl]phenyl ester), EN 100, ABT 834, ABT 239 (i.e., 4-[2-[2-[(2R)-2-methylpyrrolidinyl]ethyl]-benzofuran-5-yl-]benzonitrile), SGS 518, R 1500, C 9138, SSR 180711, alfatradiol, R 1577, T 817MA (i.e., 1-[3-[2-(1-benzothien-5-yl)ethoxy]propyl]azetidin-3-olmaleate), CNP 1061 (i.e., 4-methyl-5-(2-nitrooxyethyl)thiazole), KTX 0101 (i.e., sodium beta-hydroxybutyrate), GSK 189254 (i.e., 6-[3-cyclobutyl-2,3,4,5-tetrahydro-1H-benzo[d]azepin-7-yloxy]-N-methylnicotinamide), AZD 1080, ACC 001, PRX 07034, midazolam, R-phenserine, AZD 103 (CAS Number 488-59-5), SN 522, NGX 267 (CAS Number 503431-81-0), N-PEP-12, RN 1219, FGLL, AVE 8112, EVT 101, NP 031112, MK 0752, MK 0952, LX 6171, PAZ 417, AV 965, PF 3084014, SYN 114, GSI 953, SAM 315, SAM 531, D-serine, leteprinim potassium, BR 16A (CAS Number 149175-77-9), RPR 107393 (CAS Number 190841-57-7), NXD 2858, REN 1654, CDD 0102, NC 1900 (CAS Number 132925-74-7), ciclosporin, NCX 2216 (i.e., (E)-4-(Nitrooxy)butyl 3-[4-[2-(2-fluorobiphenyl-4-yl)propanoyloxy]-3-methoxyphenyl]acrylate), NXD 3109, NXD 1191, ZSET 845 (i.e., 3,3-diphenylimidazo[1,2-a]pyridin-2-(3H)-one), ET 002, NT 13, RO 638695 (i.e., [1,6-(1,6-dioxohexyl)]dipyrrolidine-(2R)-carboxylic acid), bisnorcymserine, BA 1016, XD 4241, EUK 207 (i.e., (SP-5-13)-(acetato-κO)[13,16,19,22-tetraoxa-3,6-diazatricyclo[21.3.18.12]octacosa-1(27),2,6,8,10,12(28),23,25-octaene-27,28-diolato(2-)-κN3,κN6,κO27,κO28]manganese), LG 617 inhibitors, ZSET 1446, PAN 811, F 14413 (i.e., 2-[5-Fluoro-2(S)-methoxy-2,3-dihydro-1,4-benzodioxin-2-yl]-4,5-dihydro-1H-imidazole), FP 7832 (i.e., N-[2-(5-Methoxy-1-nitroso-1H-indol-3-yl)ethyl]acetamide), ARA 014418 (i.e., N-(4-methoxybenzyl)-N′-(5-nitro-1,3-thiazol-2-yl)urea), AZD 3102, KP 544 (i.e., 2-amino-5-(4-chlorophenylethynyl)-4-(4-trans-hydroxycyclohexylamino)pyrimidine), DP 155, 5-Chloro-N-[3-[2-(dimethylamino)ethyl]-1H-indol-5-yl]naphthalene-2-sulfonamide, TAK 070, huperzine, N-[2-(3,5-Dimethyladamant-1-yl)ethyl]acetamidine hydrochloride, 6-[4-[(Dimethylamino)methyl]-5-ethyl-2-methoxyphenyl]pyridin-2-amine, 4,6-diphenyl-3-(4-(pyrimidin-2-yl)piperazin-1-yl)pyridazine, N-[(1S,2R)-3-(3,5-Difluorophenyl)-1-hydroxy-1-[(5S,6R)-5-methyl-6-(neopentyloxy)morpholin-3-yl]propan-2-yl]acetamide hydrochloride, N-[(1R,2S)-3-(3,5-Difluorophenyl)-1-hydroxy-1-[(2R,4R)-4-phenoxypyrrolidin-2-yl]propan-2-yl]-3-[(R)-2-(methoxymethyl)pyrrolidine-1-carbonyl]-5-methylbenzamide, R 1589, midafotel, phenserine, coluracetam, physostigmine, cipralisant, nitroflurbiprofen, PPI 1019 (i.e., (3alpha,5beta,7alpha,12alpha)-trihydroxycholan-24-oyl-L-leucyl-L-valyl-L-phenylalanyl-L-phenylalanyl-L-alanine), dapsone, MDL 100453 (CAS Number 129938-34-7), NS 377, midaxifylline, propofol phosphate, metrifonate, ceronapril, tenilsetam, sufoxazine, seglitide, ebiratide, nebracetam, milacemide, iododoxorubicin, SM 10888 (CAS Number 129297-21-8), U 80816 (CAS Number 138554-11-7), YM 954 (CAS Number 132041-85-1), SUT 8701 (CAS Number 123577-73-1), apovincamine, FR 121196 (CAS Number 133920-65-7), LY 274614 (CAS Number 136109-04-1), CL 275838 (CAS Number 115931-65-2), igmesine, K 7259 (CAS Number 133667-88-6), vinconate, itasetron, CL 287663 (CAS Number 125109-98-0), WAY 100289 (CAS Number 136013-69-9), SR 46559A (CAS Number 137733-33-6), GYKI 46903 (CAS Number 142999-59-5), L 670548 (CAS Number 121564-89-4), Y 29794 (CAS Number 129184-48-1), AF 125 (CAS Number 7631-86-9), KFM 19 (CAS Number 133058-72-7), ST 796 (i.e., (S)-3[3-(trifluoromethyl)benzoyl)amino]hexahydroazepin-2-one), RU 33965 (CAS Number 122321-05-5), SDZ 210086 (i.e., (−)-1′,2(S)-Dimethylspiro[1,3-dioxolan-4,4′-piperidine]), L 689660 (CAS Number 144860-79-7), L 689560 (CAS Number 139051-78-8), ST 618 (i.e., 1-(6,7-Dimethoxy-1,2,3,4-tetrahydro-2-naphthyl)-4-hydroxy pyrrolidin-2-one), U 74500A (CAS Number 110101-65-0), GEA 857 (CAS Number 120493-42-7), BIBN 99 (CAS Number 145301-48-0), DX 9366, ONO 1603 (CAS Number 114668-76-7), MDL 102234 (CAS Number 137766-81-5), P 9939 (CAS Number 157971-37-4), PD 140532 (CAS Number 157971-39-6), azetirelin, MR 16728 (CAS Number 147614-21-9), dabelotine, MDL 102503 (i.e., 8-[1(R)-methyl-2-phenylethyl]-1,3-dipropyl-7H-xanthine), PD 141606 (i.e., (±)-(Z)-3-(3-Phenyl-2-propynyloxyimino)-1-azabicyclo[2.2.1]heptane), SNK 882 (CAS Number 152221-12-0), L 696986 (CAS Number 141553-45-9), tazomeline, LY 235959 (CAS Number 137433-06-8), 2-(2-thiooxopyrrolidin-1-yl)acetamide, AK 30 NGF, ABT 418 (CAS Number 147402-53-7), itameline, HUP 13, sibopirdine, KST 5452 (CAS Number 157998-88-4), TJ 54, U 92798 (i.e., 7-[4-[Bis(4-fluorophenyl)methyl]perhydro-1,4-diazepin-1-ylmethyl]-4-isopropyl-2-methoxy-2,4,6-cycloheptatrien-1-one), U 92032 (CAS Number 142223-92-5), 3-(sulfamoyloxy)estra-1,3,5(10)-trien-17-one, P 11012 (CAS Number 164723-36-8), A 82695 (CAS Number 147388-86-1), FR 76659 (CAS Number 116904-25-7), apaxifylline, CX 417, 7 MEOTA (CAS Number 5778-80-3), BU 4514N(CAS Number 151013-39-7), pregnenolone, mexidol, ST 857 (CAS Number 154755-63-2), RU 49041 (CAS Number 123828-80-8), RU 35929 (CAS Number 111711-47-8), P 878184, P 128 (CAS Number 157716-52-4), eurystatin A, eurystatin B, LK 12, NBI 108, NBI 107, NBI 117, L 705106, bacoside A+B, clausenamide, SM 21 (CAS Number 155156-22-2), alaptide, RS 17017 (i.e., 1-(4-Amino-5-chloro-2-methoxyphenyl)-5-(1-piperidinyl)-1-pentanone hydrochloride), AF 150(S) (i.e., (S)-[1-Methyl-piperidine-4-spiro-(2′-methylthiazoline)]), RO 153505 (CAS Number 78771-13-8), PV 113 (i.e., 1,2,3,4-Tetrahydropyrrolo-[1,2-a]-pyrazine), arisugacin, A 98284 (i.e., 2(R)-(3-Methylisoxazol-5-yl)quinuclidine), AP 5 (CAS Number 136941-85-0), BD 1054, SDZ NDD 094 (i.e., bis-(2-(2-methylimidazol-1-yl]methyl)-pyridine-tris(hydrogen-fumarate), AZ 36041 (CAS Number 173324-76-0), quilostigmine, A 84543 (i.e., 3-[1-Methylpyrrolidin-2-(S)-ylmethoxy]pyridine fumarate), BTG 4247 (i.e., (2-[2-Chloroethoxy[4-(dimethylamino)phenyl]phosphoryl]-acetohydrazide), CGP 50068 (CAS Number 158647-49-5), cerebrocrast, desferri-nordanoxamine, isolichenan, MHP 133 (i.e., 3-(N,N-dimethylcarbamoyloxy)-1-methyl-2-(4-phenyl-semicarbazonomethyl)pyridinium chloride), FR 152558 (CAS Number 151098-08-7), GVS 111 (CAS Number 157115-85-0), P 11149 (CAS Number 164724-79-2), PDC 008004, KST 2818 (CAS Number 158623-26-8), KST 5410 (CAS Number 158623-27-9), RU 52583 (CAS Number 123829-33-4), PD 151832 (CAS Number 149929-39-5), UCL 1199 (i.e., 4-[2-[(5-nitropyridin-2-ylsulfanyl)ethyl]-1H-imidazole), isovanihuperzine A, SIB 1765F (CAS Number 179120-52-6), JWS USC 751X (i.e., 3-[[[2-[[(5-dimethylaminomethyl)-2-furanyl]methyl]thio]ethyl]amino]-4-nitropyridazine), GR 175737 (i.e., 3-(4-Chlorobenzyl)-5-[2-(1H-imidazol-4-yl)ethyl]-1,2,4-oxadiazole), KS 505A (CAS Number 131774-53-3), ZTTA 1 (i.e., N-benzyloxycarbonyl-thioprolyl-thioprolynal-dimethylaceta 1), AGN 190837 (CAS Number 136527-40-7), P 10358 (188240-59-7), WAY 131256 (CAS Number 174001-71-9), DBO 83 (i.e., 3-(6-chloropyridazin-3-yl)-diazabicyclo[3.2.1]octane dihydrochloride monohydrate), FUB 181 (CAS Number 152029-80-6), RJR 2557, WSU 2088, LVV-haemorphin-7, M 40 (i.e., galanin[1-12]-Pro3-(Ala-Leu)-2-Ala-NH₂), SIB 1757, SKF 74652 (i.e., [5-chloro-2-(4-methoxy phenyl)-3-benzofuranyl][4-[3-(diethylamino)-propoxy]phenyl]methanone), CGP 71982, SCH 57790 (i.e., 4-cyclohexyl-alpha-[4-[[4-methoxyphenyl]sulfinyl]phenyl]-1-piperazineacetonitrile), Putrescine-D-YiAbeta11, DU 14 (i.e., p-O-(sulfamoyl)-N-tetradecanoyl tyramine), CLZ 4, SL 340026, PPRT 424, ciproxifan, UR 1827 (i.e., 2-(1-benzylpiperidin-4-yl)-1-[4-(5-methylpyrimidin-4-ylamino)phenyl]-1-ethanone), caproctamine, TGS 20 (i.e., L-pyroglutamil-D-alanine amide), PG 9 (i.e., alpha-tropanyl 2-[(4-bromo)phenyl]propionate), TEI 3356 (i.e., (16S)-15-Deoxy-16-hydroxy-16-methyl-9-(O)-methano-DELTA6(9alpha)-prostaglandin I1), LY 392098 (i.e., Thiophene, 3-[(2-methylethyl-2)sulphonylaminopropyl-2]phenyl-4-yl-), PG 1000, DM 232, NEPP 11 (i.e., 12-iso-15-Deoxy-18-(4-methyl)phenyl-13,14-dihydro-delta7-prostaglandinA1 methyl ester), VA 100 (i.e., (2,3-Dihydro-2-[[(4-fluorobenzoyl)amino]ethyl]-1-methyl-5-phenyl-1H-1,4-benzodiazepine), VA 101 (i.e., (2,3-dihydro-2-[[(2-thienylcarbonyl)amino]ethyl]-1-methyl-5-phenyl-1H-1,4-benzodiazepine), NC 111585 (i.e., (3S)-1,3-Bis-[3-[(3-azabicylo[2.2.2]octanyl)-1,2,5-thiadiazol-4-yloxy]-1-propyn-1-yl]benzene, 2L-(+)-tartate), IN 201, imoproxifan, kanokodiol, picroside I, picroside II, DM 235 (i.e., 1-(4-Benzoylpiperazin-1-yl)propan-1-one), monoclonal antibody 10D5, JLK2, JLK 6, JLK 7, DAPT (i.e., N—[N-(3,5-difluorophenacetyl)-L-alanyl]-S-phenylglycine t-butyl ester), huperine X, SGS 111 (i.e., (S)-ethyl 2-[1-(2-phenylacetyl)pyrrolidine-2-carboxamido]acetate), NP 7557, C 9136, C 7617, R 1485, rofecoxib, velnacrine, montirelin, lazabemide, ORG 2766 (CAS Number 50913-82-1), sabeluzole, adafenoxate, CAS Number 9061-61-4, ipidacrine, bemesetron, idazoxan, linopirdine, selfotel, suritozole, milameline, xanomeline, TJ 960, fasoracetam, eptastigmine, ensaculin, zanapezil, posatirelin, zacopride, RS 86 (CAS Number 3576-73-6), ORG 5667 (CAS Number 37552-33-3), RX 77368 (CAS Number 76820-40-1), BMS 181168 (CAS Number 123259-91-6), BY 1949 (CAS Number 90158-59-1), AWD 5239 (CAS Number 109002-93-9), YM 796 (171252-79-2), aloracetam, CI-933 (CAS Number 91829-95-7), ST 793 (CAS Number 99306-37-3), cebaracetam, zifrosilone, talsaclidine, alvameline, JTP 2942 (148152-77-6), OPC 14117 (CAS Number 103233-65-4), elziverine, AP 521 (i.e., N-(1,3-Benzodioxol-5-ylmethyl)-1,2,3,4-tetrahydro[1]benzothieno[2,3-c]pyridine-3(R)-carboxamide hydrochloride), S 8510 (CAS Number 151466-23-8), JTP 4819 (CAS Number 162203-65-8), icopezil, SC 110, FK 960 (CAS Number 133920-70-4), DMP 543 (CAS Number 160588-45-4), ganstigmine, CI 1017 (i.e., (R)-(−)-(Z)-1-Azabicyclo[2.2.1]heptan-3-one, 0-(3-(3′-methoxyphenyl)-2-propynyl)-oxime maleate), T 82 (i.e., 2-[2-(1-Benzylpiperidin-4-yl)ethyl]-2,3-dihydro-9-methoxy-1H-pyrrolo[3,4-b]quinolin-1-one hemifumarate), NGD 971, vaccine of Aspartyl-alanyl-glutamyl-phenylalanyl-arginyl-histidyl-aspartyl-seryl-glycyl-tyrosyl-glutamyl-valyl-histidyl-histidyl-glutaminyl-lysyl-leucyl-valyl-phenylalanyl-phenylalanyl-alanyl-glutamyl-aspartyl-valyl-glycyl-seryl-asparaginyl-lysyl-glycyl-alanyl-isoleucyl-isoleucyl-glycyl-leucyl-methionyl-valyl-glycyl-glycyl-valyl-valyl-isoleucyl-alanine, PBT 1 (CAS Number 130-26-7), TCH 346, FK 962 (i.e., N-(1-acetylpiperidin-4-yl)-4-fluorobenzamide), voxergolide, KW 6055 (CAS Number 63233-46-5), thiopilocarpine, ZK 93426 (CAS Number 89592-45-0), SDZ NVI 085 (CAS Number 104195-17-7), CI 1002 (CAS Number 149028-28-4), Z 321 (CAS Number 130849-58-0), mirisetron, CHF 2060 (i.e., N-Heptylcarbamic acid 2,4a,9-trimethyl-2,3,4,4a,9,9a-hexahydro-1,2-oxazino[6,5-b]indol-6-yl ester-L-tartrate), gedocarnil, terbequinil, HOE 065 (CAS Number 123060-44-6), SL 650102, GR 253035, ALE 26015, SB 271046 (i.e., 5-Chloro-N-(4-methoxy-3-piperazin-1-yl-phenyl)-3-methyl-2-benzothiophenesulfonamide), iAbeta5, SCH 211803 (i.e., Piperidine, 1-[1-(3-methyl-2-aminophenyl)carbonylpiperidin-4-yl]-4-[(3-chlorophenypsulphonylphenyl-4]methyl-), EVT 301, alpha-Linolenic acid/linoleic acid, Kamikihi-To, siagoside, FG 7142 (CAS Number 78538-74-6), RU 47067 (CAS Number 111711-92-3), RU 35963 (CAS Number 139886-03-6), FG 7080 (CAS Number 100332-18-1), E 2030 (CAS Number 142007-70-3), transforming growth factor beta-1, A 72055 (i.e., 2′,1-Dimethylspiro[piperidine-4,5′ oxazolidine]-3′-carboxaldehyde), NS 626, dimiracetam, GT 3001, GT 2501, GT 2342, GT 2016 (CAS Number 152241-24-2), ORG 20091 (CAS Number 141545-50-8), BCE 001 (CAS Number 95678-81-2), CGP 35348 (CAS Number 123690-79-9), WAY 100635 (CAS Number 146714-97-8), E 4804 (CAS Number 162559-34-4), LIGA 20 (CAS Number 126586-85-4), NG 121 (i.e., 2-[4,8-Dimethyl-3(E),7(E)-nonadienyl]-3,5-dihydroxy-2-methyl-3,4,7,9-tetrahydro-2H-furo[3,4-h]-1-benzopyran-7-one), MF 247 (i.e., N-[10-(Diethylamino)decyl]carbamic acid (3aS,8aR)-1,3a,8-trimethyl-1,2,3,3a,8,8a-hexahydropyrrolo[2,3-b]indol-5-yl ester), JTP 3399 (i.e., N-Benzyl-2(S)-[2(S)-(phenoxyacetyl)pyrrolidin-1-ylcarbonyl]pyrrolidine-1-carboxamide), KF 17329, thioperamide, F 3796 (i.e., 1-[2-(1-Benzylpiperidin-4-yl)ethyl]-3-[3,4-(methylene-dioxy)benzoyl]thiourea), GT 4001, GT 4002, FPL 14995 (CAS Number 123319-03-9), RU 34332 (CAS Number 137157-58-5), SR 96777A (CAS Number 115767-94-7), SIB T1980, NS 649 (CAS Number 146828-02-6), PD 142505 (CAS Number 149929-08-8), GYKI 52466 (CAS Number 102771-26-6), RO 246173 (CAS Number 159723-57-6), SCH 50911 (CAS Number 160415-07-6), Z 4105 (CAS Number 119737-52-9), RS 67333 (CAS Number 168986-60-5), NS 1546, ZM 241385 (CAS Number 139180-30-6), RO 249975 (i.e., [1S,3S(2′S),5R]-3-(1-Benzyl-5-oxopyrrolidin-2-ylmethyl)-5-(1H-imidazol-5-ylmethyl)cyclohexane-1-acetamide), AF 185 (i.e., 8-Methyl-3-(2-propynyl)-1,3,8-triazaspiro[4,5]decane-2,4-dione), CEP 427, CX 423, CX 438, CX 480, CDP-ethanolamine, GT 4003, GT 4011, GT 5011, MS 430 (CAS Number 122113-44-4), MBF 379 (i.e., [3,3-Bis(hydroxymethyl)-8-hydroxy-3,4-dihydro-2H-1,4-benzoxazin-5-yl][3′,5′-dihydroxy-4′-(2-oxo-2-phenylethoxy)phenyl]methanone), NGD 187 (CAS Number 163565-48-8), DUP 856, MR 3066, MF 8615 (i.e., 5-Amino-6-chloro-4-hydroxy-3,4-dihydro-1H-thiopyrano-[3,4-b]quinoline), himbacine, ABS 300, RJR 2403 (CAS Number 538-79-4), MF 268 (CAS Number 174721-00-7), RO 465934 (i.e., N,N-Dimethylcarbamic acid 3-(2-cyclohexylethyl)-2,3,3a,4,5,9b-hexahydro-1H-benz[e]indol-6-yl ester), NS 393, RGH 2716 (CAS Number 134069-68-4), WIN 678702 (12,12-Bis(3-furyl)-6,11-dihydro-6,11-ethanobenzo[b]quinolizinium chloride), RS 66252 (i.e., 1-Butyl-2-[(2′-(2H-tetrazol-5-yl)-biphenyl-4-yl)methyl]-1H-indole-3-carboxylic acid), AIT 034 (CAS Number 138117-48-3), NG 012 (CAS Number 131774-53-3), PD 142012 (CAS Number 5778-84-7), GT 4054, GT 4077, GT 4035, P 26 (CAS Number 152191-74-7), RGH 5279 (i.e., (−)-(13aR,13bS)-13a-Ethyl-2,3,5,6,13a,13b-hexahydro-1H-indolo[3,2,1-de]pyrido[3,2,1-ij][1,5]naphthyridine-12-carboxylic acid 2-acetoxyethyl ester), AIT 083, CeNeS, estradiol (i.e., 1,3,5(10)-Estratriene-3,17beta-diol), WAY 132983 ((3R,4R)-3-(3-hexylsulfanylpyrazin-2-yloxy)-1-azabicyclo[2.2.1]heptane hydrochloride), ABS 205, ABS 401, SX 3507 (i.e., 3-(3-Propyl-1,2,4-oxadiazol-5-yl)quinoxaline-2(1H)-one), ARR 17779 (i.e., (−)-Spiro[1-azabicyclo[2.2.2]octane-3,5-oxazolidine]-2-one), XE 991 (i.e., 10,10-bis(4-Pyridylmethyl)anthracen-10(9H)-one), phenethylnorcymserine, RO 657199, RJR 1781 (i.e., R(+)-2-(3-pyridyl)-1-azabicyclo[2.2.2.]octane), RJR 1782 (i.e., S(+2-(3-pyridyl)-1-azabicyclo[2.2.2.]octane), gilatide, tolserine, TC 2559 (i.e., (E)-N-Methyl-4-[3-(5-ethoxypyridin)yl]-3-buten-1-amine), ER 127528 (i.e., 1-(3-Fluorobenzyl)-4-[(2-fluoro-5,6-dimethoxy-1-indanone-2-yl)methyl]piperidine hydrochloride), thiatolserine, targacept, axonyx, cymserine, thiacymserine, monoclonal antibody 266, Apan-CH, DP 103, SPI 339 (i.e., 4-[3-(4-Oxo-4,5,6,7-tetrahydroindol-1-yl)propionylamino]benzoic acid ethyl ester), S 37245 (i.e., 4-(1,4-Benzodioxan-5-yl)-1-[3(S)-hydroxy-5-nitro-indan-2-yl]-piperazine), LLG 88, AZD 2858, trometamol, AN 240, NG 002 (i.e., 5-Hydroxy-5-(2-hydroxy-1-methylethyl)-4-methoxyfuran-2(5H)-one), UCB 29427 (i.e., 2-Cyclopropyl-4-(cyclopropylamino)-6-(morpholino)-1,3,5-triazine), TRH-SR, RO 401641 (CAS Number 122199-02-4), MPV 1743AIII (CAS Number 150586-64-4), IDRA 21 (CAS Number 22503-72-6), CEP 431, ACPD (CAS Number 67684-64-4), CT 3577 (i.e., 3,7-Dimethyl-1-[11-(3,4,5-trimethoxybenzylamino)-11-oxoundecyl]xanthine), CT 2583, and NXD 9062.

The cinnamide compounds, all of which are active ingredients, may be any known in the art. In one embodiment, the cinnamide compounds are the compounds described in US Publication No. 2006/0004013, and the corresponding PCT Publication No. WO 2005/115990. In one embodiment, the cinnamide compound is a compound of Formula (I) or a pharmaceutically acceptable salt thereof:

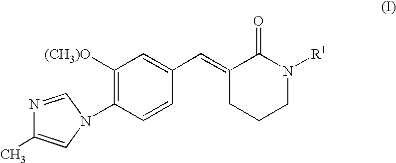

where R¹ is:

(1) —X₁—Ar₁
wherein X₁ is a C₁₋₆ alkylene group optionally substituted with a C₁₋₆ alkyl group;
and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

(2) an indenyl group optionally substituted with 1 to 3 halogen atoms;

(3) a tetrahydronaphthyl group optionally substituted with 1 to 3 halogen atoms; or

(4) a chromanyl group optionally substituted with 1 to 3 halogen atoms.

“Halogen atom” refers to a fluorine atom, a chlorine atom, a bromine atom, and an iodine atom. In one embodiment, the halogen atom is a fluorine atom, a chlorine atom, or a bromine atom.

“C₁₋₆ alkyl group” refers to a linear or branched alkyl group having 1 to 6 carbon atoms. Exemplary C₁₋₆ alkyl groups include methyl, ethyl, n-propyl, iso-propyl, n-butyl, iso-butyl, tert-butyl, n-pentyl, iso-pentyl, neopentyl, n-hexyl, 1-methylpropyl, 1,2-dimethylpropyl, 1-ethylpropyl, 1-methyl-2-ethylpropyl, 1-ethyl-2-methylpropyl, 1,1,2-trimethylpropyl, 1-methylbutyl, 2-methylbutyl, 1,1-dimethylbutyl, 2,2-dimethylbutyl, 2-ethylbutyl, 1,3-dimethylbutyl, 2-methylpentyl, 3-methylpentyl group, and the like.

In other embodiments, the compound of Formula (I) is (E)-1-(3,4-difluorobenzyl)-3-[3-methoxy-4-(4-methyl-1H-imidazol-1-yl)benzylidene]piperidin-2-one or a pharmaceutically acceptable salt thereof; (E)-1-indan-2-yl-3-[3-methoxy-4-(4-methyl-1H-imidazol-1-yl)benzylidene]piperidin-2-one or a pharmaceutically acceptable salt thereof; (E)-1-[(4R)-chroman-4-yl]-3-[3-methoxy-4-(4-methyl-1H-imidazol-1-yl)benzylidene]piperidin-2-one or a pharmaceutically acceptable salt thereof; (E)-1-[(4S)-chroman-4-yl]-3-[3-methoxy-4-(4-methyl-1H-imidazol-1-yl)benzylidene]piperidin-2-one or a pharmaceutically acceptable salt thereof; (E)-1-[(1S)-1-(4-fluorophenyl)ethyl]-3-[3-methoxy-4-(4-methyl-1H-imidazol-1-yl)benzylidene]piperidin-2-one or a pharmaceutically acceptable salt thereof; (E)-1-(4-tert-butylbenzyl)-3-[3-methoxy-4-(4-methyl-1H-imidazol-1-yl)benzylidene]piperidin-2-one or a pharmaceutically acceptable salt thereof; (E)-1-[(2S)-5-fluoroindan-2-yl]-3-[3-methoxy-4-(4-methyl-1H-imidazol-1-yl)benzylidene]piperidin-2-one or a pharmaceutically acceptable salt thereof; (E)-1-[(2R)-5-fluoroindan-2-yl]-3-[3-methoxy-4-(4-methyl-1H-imidazol-1-yl)benzylidene]piperidin-2-one or a pharmaceutically acceptable salt thereof; (E)-1-[(4R)-7-fluorochroman-4-yl]-3-[3-methoxy-4-(4-methyl-1H-imidazol-1-yl)benzylidene]piperidin-2-one or a pharmaceutically acceptable salt thereof; (E)-1-[(4S)-7-fluorochroman-4-yl]-3-[3-methoxy-4(4-methyl-1H-imidazol-1-yl)benzylidene]piperidin-2-one or a pharmaceutically acceptable salt thereof; (E)-3-[3-methoxy-4-(4-methyl-1H-imidazol-1-yl)benzylidene]-1-[(2R)-1,2,3,4-tetrahydronaphthalen-2-yl]piperidin-2-one or a pharmaceutically acceptable salt thereof; (E)-3-[3-methoxy-4-(4-methyl-1H-imidazol-1-yl)benzylidene]-1-[(2S)-1,2,3,4-tetrahydronaphthalen-2-yl]piperidin-2-one or a pharmaceutically acceptable salt thereof; (E)-1-[(1R)-1-(2,4-difluorophenyl)ethyl]-3-[3-methoxy-4-(4-methyl-1H-imidazol-1-yl)benzylidene]piperidin-2-one or a pharmaceutically acceptable salt thereof; (E)-1-[(1S)-1-(2,4-difluorophenyl)ethyl]-3-[3-methoxy-4-(4-methyl-1H-imidazol-1-yl)benzylidene]piperidin-2-one or a pharmaceutically acceptable salt thereof.

Methods for administering, dosing, and preparing cinnamide compounds are described in US Publication No. 2006/0004013, and PCT Publication No. WO 2005/115990.

The cholinesterase inhibitor, an active ingredient, used in the methods and pharmaceutical compositions of the invention can be any in the art. Exemplary cholinesterase inhibitors include donepezil, phenserine, tolserine, phenethylnorcymserine, ganstigmine, epastigmine, tacrine, physostigmine, pyridostigmine, neostigmine, rivastigmine, galantamine, citicoline, velnacrine, huperzine (e.g., huperzine A), metrifonate, heptastigmine, edrophonium, TAK-147 (i.e., 3-[1-(phenylmethyl)-4-piperidinyl]-1-(2,3,4,5-tetrahydro-1H-1-benzazepin-8-yl)-1-propanone fumarate or other salts thereof), T-82, upreazine, and the like.

In one embodiment, the cholinesterase inhibitor is a compound of Formula (II) or a pharmaceutically acceptable salt thereof:

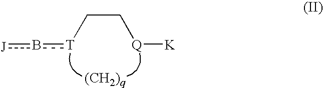

wherein J is
(a) a substituted or unsubstituted group selected from the group consisting of (1) phenyl, (2) pyridyl, (3) pyrazyl, (4) quinolyl, (5) cyclohexyl, (6) quinoxalyl, and (7) furyl;
(b) a monovalent or divalent group, in which the phenyl can have one or more substituents selected from (1) indanyl, (2) indanonyl, (3) indenyl, (4) indenonyl, (5) indanedionyl, (6) tetralonyl, (7) benzosuberonyl, (8) indanolyl, and (9) C₆H₅—CO—CH(CH₃)—;
(c) a monovalent group derived from a cyclic amide compound;
(d) a lower alkyl group; or
(e) a group of R²¹—CH═CH—, in which R²¹ is hydrogen or a lower alkoxycarbonyl group;

B is —(CHR²²)ᵣ—, —CO—(CHR²²)ᵣ—, —NR⁴—(CHR²²)—, —CO—NR⁵—(CHR²²)ᵣ—, —CH═CH—(CHR²²)ᵣ—, —OCOO—(CHR²²)ᵣ—, —OOC—NH—(CHR²²)ᵣ—, —NH—OO—(CHR²²)ᵣ—, —CH₂—CO—NH—(CHR²²)ᵣ—, —(CH₂)₂—NH—(CHR²²)ᵣ—, —CH(OH)—(CHR²²)ᵣ—, ═(CH—CH═CH)_{b}—, ═CH—(CH₂)_{c}—, ═(CH—CH)_{d}═, —CO—CH═CH—CH₂—, —CO—CH₂—CH(OH)—CH₂—, —CH(CH₃)—CO—NH—CH₂—, —CH═CH═CO—NH—(CH₂)₂—, —NH—, —O—, —S—, a dialkylaminoalkyl-carbonyl or a lower alkoxycarbonyl;

wherein R⁴ is hydrogen, lower alkyl, acyl, lower alkylsulfonyl, phenyl, substituted phenyl, benzyl, or substituted benzyl; R⁵ is hydrogen, lower alkyl or phenyl; r is zero or an integer of about 1 to about 10; R²² is hydrogen or methyl so that one alkylene group can have no methyl branch or one or more methyl branches; b is an integer of about 1 to about 3; c is zero or an integer of about 1 to about 9; d is zero or an integer of about 1 to about 5;

T is nitrogen or carbon;

Q is nitrogen, carbon or

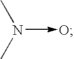

q is an integer of about 1 to about 3;

K is hydrogen, phenyl, substituted phenyl, arylalkyl in which the phenyl can have a substituent, cinnamyl, a lower alkyl, pyridylmethyl, cycloalkylalkyl, adamantanemethyl, furylmethyl, cycloalkyl, lower alkoxycarbonyl or an acyl; and

[symbol] is a single bond or a double bond.

In the compound of Formula (II), J is preferably (a) or (b), more preferably (b). In the definition of (b), a monovalent group (2), (3) and (5) and a divalent group (2) are preferred. The group (b) preferably includes, for example, the groups having the formulae shown below:

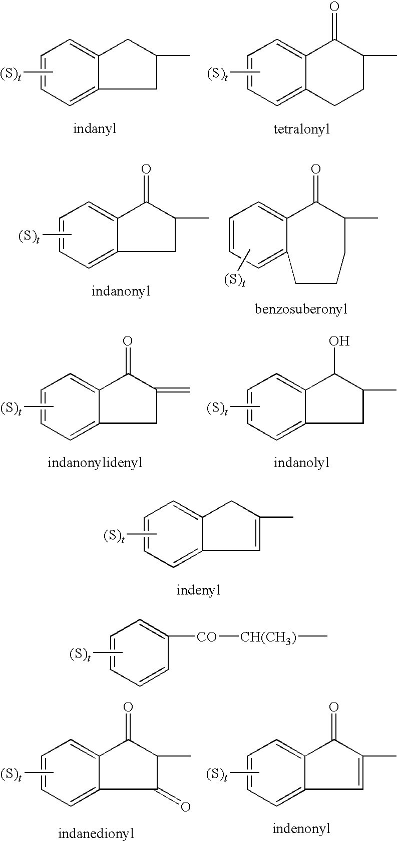

wherein t is an integer of about 1 to about 4; and each S is independently hydrogen or a substituent, such as a lower alkyl having 1 to 6 carbon atoms or a lower alkoxy having 1 to 6 carbon atoms. Among the substituents, methoxy is most preferred. The phenyl is most preferred to have 1 to 3 methoxy groups thereon. (S)ₜ can form methylene dioxy groups or ethylene dioxy groups on two adjacent carbon atoms of the phenyl group. Of the above groups, indanonyl, indanedionyl and indenyl, optionally having substituents on the phenyl, are the most preferred.

In the definition of B, —(CHR²²)ᵣ—, —CO—(CH—CH═CH)_{b}—, ═CH—(CH₂)_{c}— and ═(CH—CH)_{d}═ are preferable. The group of —(CHR²²)ᵣ— in which R²² is hydrogen and r is an integer of 1 to 3, and the group of ═CH—(CH₂)_{c}— are most preferable. The preferable groups of B can be connected with (b) of J, in particular (b)(2).

The ring containing T and Q in Formula (II) can be 5-, 6- or 7-membered. It is preferred that Q is nitrogen, T is carbon or nitrogen, and q is 2; or that Q is nitrogen, T is carbon, and q is 1 or 3; or that Q is carbon, T is nitrogen and q is 2.

It is preferable that K is a phenyl, arylalkyl, cinnamyl, phenylalkyl or a phenylalkyl having a substituent(s) on the phenyl.

In another embodiment, the cyclic amine compounds of Formula (II) are the piperidine compounds of Formula (III) or a pharmaceutically acceptable salt thereof:

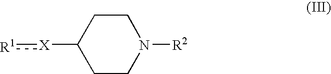

wherein R¹ is a (1) substituted or unsubstituted phenyl group; (2) a substituted or unsubstituted pyridyl group; (3) a substituted or unsubstituted pyrazyl group; (4) a substituted or unsubstituted quinolyl group; (5) a substituted or unsubstituted indanyl group; (6) a substituted or unsubstituted cyclohexyl group; (7) a substituted or unsubstituted quinoxalyl group; (8) a substituted or unsubstituted furyl group; (9) a monovalent or divalent group derived from an indanone having a substituted or unsubstituted phenyl ring; (10) a monovalent group derived from a cyclic amide compound; (11) a lower alkyl group; or (12) a group of the formula R³—CH═C—, where R³ is a hydrogen atom or a lower alkoxycarbonyl group;

X is —(CH₂)—, —C(O)—(CH₂)ₙ—, —N(R⁴)—(CH₂)ₙ—, —C(O)—N(R⁵)—(CH₂)ₙ—, —CH═CH—(CH₂)ₙ—, —O—C(O)—O—(CH₂)ₙ—, —O—C(O)—NH—(CH₂)ₙ—, —CH═CH—CH═CO—, —NH—C(O)—(CH₂)ₙ—, —CH₂—C(O)—NH—(CH₂)ₙ—, —(CH₂)₂—C(O)—NH—(CH₂)ₙ—, —CH(OH)—(CH₂)ₙ—, —C(O)—CH═CH—CH₂—, —C(O)—CH₂—CH(OH)—CH₂—, —CH(CH₃)—C(O)—NH—CH₂—, —CH═CH—C(O)—NH—(CH₂)₂—, a dialkylaminoalkylcarbonyl group, a lower alkoxycarbonyl group;

where n is an integer of 0 to 6; R⁴ is a hydrogen atom, a lower alkyl group, an acyl group, a lower alkylsulfonyl group, a substituted or unsubstituted phenyl group, or a substituted or unsubstituted benzyl group; and R⁵ is a hydrogen atom a lower alkyl group or a phenyl group;

R² is a substituted or unsubstituted phenyl group; a substituted or unsubstituted arylalkyl group; a cinnamyl group; a lower alkyl group; a pyridylmethyl group; a cycloalkylalkyl group; an adamantanemethyl group; or a furylmethyl group; and

[symbol] is a single bond or a double bond.

The term “lower alkyl group” as used herein means a straight or branched alkyl group having 1 to 6 carbon atoms. Exemplary “lower alkyl groups” include methyl, ethyl, propyl, isopropyl, butyl, isobutyl, sec-butyl, tert-butyl, pentyl (amyl), isopentyl, neopentyl, tert-pentyl, 1-methylbutyl, 2-methylbutyl, 1,2-dimethylpropyl, hexyl, isohexyl, 1-methylpentyl, 2-methyl-pentyl, 3-methylpentyl, 1,1-dimethylbutyl, 1,2-dimethylbutyl, 2,2-dimethylbutyl, 1,3-dimethyl-butyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and the like. The lower alkyl group is preferably methyl, ethyl, propyl or isopropyl; more preferably methyl.

Specific examples of the substituents for the substituted or unsubstituted phenyl, pyridyl, pyrazyl, quinolyl, indanyl, cyclohexyl, quinoxalyl and furyl groups in the definition of R¹ include lower alkyl groups having 1 to 6 carbon atoms, such as methyl, ethyl, n-propyl, isopropyl, n-butyl, isobutyl, and tert-butyl groups; lower alkoxy groups corresponding to the above-described lower alkyl groups, such as methoxy and ethoxy groups; a nitro group; halogen atoms, such as chlorine, fluorine and bromine; a carboxyl group; lower alkoxycarbonyl groups corresponding to the above-described lower alkoxy groups, such as methoxycarbonyl, ethoxycarbonyl, isopropoxycarbonyl, n-propoxycarbonyl, and n-butyloxycarbonyl groups; an amino group; a lower monoalkylamino group; a lower dialkylamino group; a carbamoyl group; acylamino groups derived from aliphatic saturated monocarboxylic acids having 1 to 6 carbon atoms, such as acetylamino, propionylamino, butyrylamino, isobutyrylamino, valerylamino, and pivaloylamino groups; cycloalkyloxycarbonyl groups, such as a cyclohexyloxycarbonyl group; lower alkylaminocarbonyl groups, such as methylaminocarbonyl and ethylaminocarbonyl groups; lower alkylcarbonyloxy groups corresponding to the above-defined lower alkyl groups, such as methylcarbonyloxy, ethylcarbonyloxy, and n-propylcarbonyloxy groups; halogenated lower alkyl groups, such as a trifluoromethyl group; a hydroxyl group; a formyl group; and lower alkoxy lower alkyl groups, such as ethoxymethyl, methoxymethyl and methoxyethyl groups. The “lower alkyl groups” and “lower alkoxyl groups” in the above description of the substituent include all the groups derived from the above-mentioned groups. The substituent can be one to three of them, which can be the same or different.

When the substituent is a phenyl group, the following group is within the scope of the substituted phenyl group:

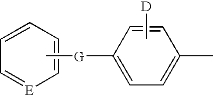

wherein G is —C(O)—, —O—C(O)—, —O—, —CH₂—NH—C(O)—, —CH₂—O—, —CH₂—SO₂—, —CH(OH)—, or —CH₂—S(→O)—; E is a carbon or nitrogen atom; and D is a substituent.

Preferred examples of the substituents (i.e., “D”) for the phenyl group include lower alkyl, lower alkoxy, nitro, halogenated lower alkyl, lower alkoxycarbonyl, formyl, hydroxyl, and lower alkoxy lower alkyl groups, halogen atoms, and benzoyl and benzylsulfonyl groups. The substituent can be two or more of them, which can be the same or different.

Preferred examples of the substituent for the pyridyl group include lower alkyl and amino groups and halogen atoms.

Preferred examples of the substituent for the pyrazyl group include lower alkoxycarbonyl, carboxyl, acylamino, carbamoyl, and cycloalkyloxycarbonyl groups.

With respect to R¹, the pyridyl group is preferably a 2-pyridyl, 3-pyridyl, or 4-pyridyl group; the pyrazyl group is preferably a 2-pyrazinyl group; the quinolyl group is preferably a 2-quinolyl or 3-quinolyl group; the quinoxalinyl group is preferably a 2-quinoxalinyl or 3-quinoxalinyl group; and the furyl group is preferably a 2-furyl group.

Specific examples of preferred monovalent or divalent groups derived from an indanone having an unsubstituted or substituted phenyl ring include those represented by formulas (A) and (B):

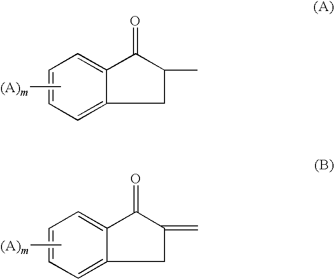

where m is an integer of from 1 to 4, and each A is independently a hydrogen atom, a lower alkyl group, a lower alkoxy group, a nitro group, a halogen atom, a carboxyl group, a lower alkoxycarbonyl group, an amino group, a lower monoalkylamino group, a lower dialkylamino group, a carbamoyl group, an acylamino group derived from aliphatic saturated monocarboxylic acids having 1 to 6 carbon atoms, a cycloalkyloxycarbonyl group, a lower alkylaminocarbonyl group, a lower alkylcarbonyloxy group, a halogenated lower alkyl group, a hydroxyl group, a formyl group, or a lower alkoxy lower alkyl group; preferably a hydrogen atom, a lower alkyl group or a lower alkoxy group; most preferably the indanone group is unsubstituted or substituted with 1 to 3 methoxy groups.

Examples of the monovalent group derived from a cyclic amide compound include quinazolone, tetrahydroisoquinolinone, tetrahydrobenzodiazepinone, and hexahydrobenzazocinone. However, the monovalent group can be any one having a cyclic amide group in the structural formula thereof, and is not limited to the above-described specific examples. The cyclic amide group can be one derived from a monocyclic or condensed heterocyclic ring. The condensed heterocyclic ring is preferably one formed by condensation with a phenyl ring. In this case, the phenyl ring can be substituted with a lower alkyl group having 1 to 6 carbon atoms, preferably a methyl group, or a lower alkoxy group having 1 to 6 carbon atoms, preferably a methoxy group.

Preferred examples of the monovalent group include the following:

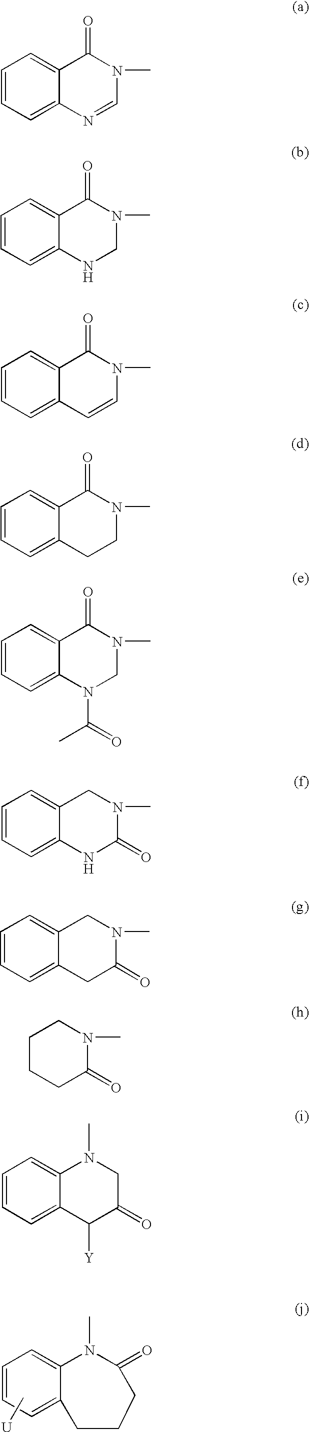

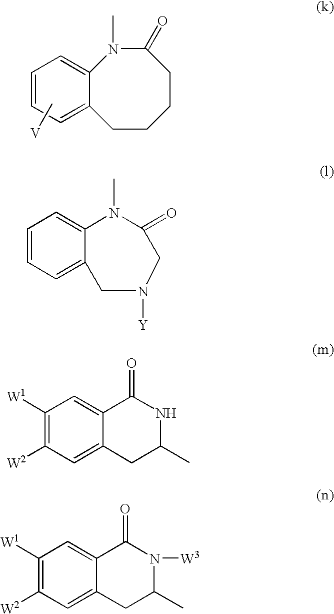

In the above formulae, Y is a hydrogen atom or a lower alkyl group; V and U are each a hydrogen atom or a lower alkoxy group (preferably dimethoxy); W¹ and W² are each a hydrogen atom, a lower alkyl group, or a lower alkoxy group; and W³ is a hydrogen atom or a lower alkyl group. The right hand ring in formulae (j) and (l) is a 7-membered ring, while the right hand ring in formula (k) is an 8-membered ring.

The most preferred examples of the above-defined R¹ include a monovalent group derived from an indanone having an unsubstituted or substituted phenyl group and a monovalent group derived from a cyclic amide compound.

The most preferred examples of the above-defined X include —(CH₂)ₙ—, an amide group, or groups represented by the above formulae where n is 2. Thus, it is most preferred that any portion of a group represented by the formula R¹ [symbol] X— have a carbonyl or amide group.

The substituents involved in the expressions “a substituted or unsubstituted phenyl group” and “a substituted or unsubstituted arylalkyl group” in the above definition of R² are the same substituents as those described for the above definitions of a phenyl group, a pyridyl group, a pyrazyl group, a quinolyl group, an indanyl group, a cyclohexyl group, a quinoxalyl group or a furyl group in the definition of R¹.

The term “arylalkyl group” is intended to mean an unsubstituted benzyl or phenethyl group or the like.

Specific examples of the pyridylmethyl group include 2-pyridylmethyl, 3-pyridylmethyl, and 4-pyridylmethyl groups.

Preferred examples of R² include benzyl and phenethyl groups. The symbol [symbol] means a double or single bond. The bond is a double bond only when R¹ is the divalent group (B) derived from an indanone having an unsubstituted or substituted phenyl ring, while it is a single bond in other cases.

In another embodiment, the compound of formula II is a compound of Formula (IV) or a pharmaceutically acceptable salt thereof:

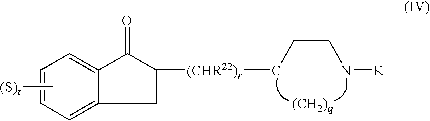

wherein r is an integer of about 1 to about 10; each R²² is independently hydrogen or methyl; K is a phenalkyl or a phenalkyl having a substituent on the phenyl ring; each S is independently a hydrogen, a lower alkyl group having 1 to 6 carbon atoms or a lower alkoxy group having 1 to 6 carbon atoms; t is an integer of 1 to 4; q is an integer of about 1 to about 3; with the proviso that (S)ₜ can be a methylenedioxy group or an ethylenedioxy group joined to two adjacent carbon atoms of the phenyl ring.

In other embodiments, the cholinesterase inhibitor is 1-benzyl-4-((5,6-dimethoxy-1-indanon)-2-yl)methylpiperidine; 1-benzyl-4-((5,6-dimethoxy-1-indanon)-2-ylindenyl)methylpiperidine; 1-benzyl-4-((5-methoxy-1-indanon)-2-yl)methylpiperidine; 1-benzyl-4-((5,6-diethoxy-1-indanon)-2-yl)methylpiperidine; 1-benzyl-4-((5,6-methylenedioxy-1-indanon)-2-yl)methylpiperidine; 1-(m-nitrobenzyl)-4-((5,6-dimethoxy-1-indanon)-2-yl)methylpiperidine; 1-cyclohexylmethyl-4-((5,6-dimethoxy-1-indanon)-2-yl)methylpiperidine; 1-(m-fluorobenzyl)-4-((5,6-dimethoxy-1-indanon)-2-yl)methylpiperidine; 1-benzyl-4-((5,6-dimethoxy-1-indanon)-2-yl)propylpiperidine; 1-benzyl-4-((5-isopropoxy-6-methoxy-1-indanon)-2-yl)methylpiperidine; 1-benzyl-44(5,6-dimethoxy-1-oxoindanon)-2-yl)propenylpiperidine; or a stereoisomer and/or a pharmaceutically acceptable salt thereof.

In still other embodiments, the cholinesterase inhibitor is donepezil (i.e., 1-benzyl-4-((5,6-dimethoxy-1-indanon)-2-yl)methylpiperidine) or a pharmaceutically acceptable salt thereof (e.g., hydrochloride). Donepezil hydrochloride is commercially available as ARICEPT® (Eisai Inc., Teaneck, N.J.).

In one embodiment, the AMPA receptor antagonist used in the methods and compositions described herein may be any known in the art. Exemplary AMPA receptor antagonists, all of which are active ingredients, include 1,2-dihydropyridine compounds, quinoxalinedione aminoalkylphosphonates, and the like.

In one embodiment, the AMPA receptor antagonist is a 1,2-dihydropyridine compound. The 1,2-dihydropyridine compound used in the methods and compositions described herein may be any known in the art. “1,2-dihydropyridine compound” includes 1,2-dihydropyridine compounds, pharmaceutically acceptable salts of 1,2-dihydropyridine compounds, stereoisomers of 1,2-dihydropyridine compounds, pharmaceutically acceptable salts of stereoisomers of 1,2-dihydropyridine compounds, hydrates of 1,2-dihydropyridine compounds, hydrates of pharmaceutically acceptable salts of 1,2-dihydropyridine compounds, stereoisomers of hydrates of 1,2-dihydropyridine compounds, and stereoisomer of hydrates of pharmaceutically acceptable salts of 1,2-dihydropyridine compounds.

The 1,2-dihydropyridine compound used in the methods and compositions described herein may be a compound of Formula (V):

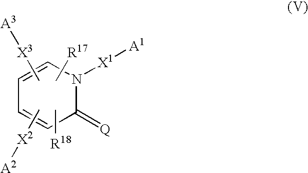

wherein Q is NH, O or S; X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

The invention provides the compound of Formula (V) wherein X¹, X² and X³ are each independently a single bond, an optionally substituted C₁₋₆ alkylene, an optionally substituted C₂₋₆ alkenylene, or an optionally substituted C₂₋₆ alkynylene. The substituents may be one or more of —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)—N(R¹²)—, —CH₂—S(O)ₚ—, —S(O)_{q}—CH₂—, —CH₂—O—, —O—CH₂—, —N(R¹³)—CO—N(R¹⁴)— and —N(R¹⁵)—CS—N(R¹⁶)—; R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring.

The substituents for the 1,2-dihydropyridine compounds of the invention may be one or more of hydroxy; halogen; nitrile; nitro; C₁₋₆ alkyl; C₂₋₆ alkenyl; C₂₋₆ alkynyl [wherein the alkyl, alkenyl, and alkynyl can independently and optionally be substituted with one or more groups selected from hydroxy, nitrile, halogen, C₁₋₆ alkylamino, di(C₁₋₆ alkyl)amino, C₂₋₆ alkenylamino, di(C₂₋₆ alkenylamino, C₂₋₆ alkynylamino, di(C₂₋₆ alkynyl)amino, N—C₁₋₆ alkyl-N—C₂₋₆ alkenylamino, N—C₁₋₆ alkyl-N—C₂₋₆ alkynylamino, N—C₂₋₆ alkenyl-N—C₂₋₆alkynylamino, aralkyloxy, TBDMS oxy, C₁₋₆ alkylsulfonylamino, C₁₋₆ alkylcarbonyloxy, C₂₋₆ alkenylcarbonyloxy, C₂₋₆ alkynylcarbonyloxy, N—C₁₋₆ alkylcarbamoyl, N—C₂₋₆ alkenylcarbamoyl, and N—C₁₋₆ alkynylcarbamoyl]; C₁₋₆ alkoxy; C₂₋₆ alkenyloxy; C₂₋₆ alkynyloxy [wherein the alkoxy, alkenyloxy, and alkynyloxy may independently and optionally be substituted with one or more groups selected from C₁₋₆ alkylamino, aralkyloxy, and hydroxy]; C₁₋₆ alkylthio; C₂₋₆ alkenylthio; C₂₋₆ alkynylthio [wherein the alkylthio, alkenylthio, and alkynylthio may independently and optionally be substituted with one or more groups selected from hydroxy, nitrile, halogen, C₁₋₆ alkylamino, aralkyloxy, TBDMS oxy, C₁₋₆ alkylsulfonylamino, C₁₋₆ alkylcarbonyloxy, and C₁₋₆ alkylcarbamoyl]; optionally substituted carbonyl [which may be substituted with C₁₋₆ alkoxy, amino, C₁₋₆ alkylamino, di(C₁₋₆ alkyl)amino, C₂₋₆ alkenylamino, di(C₂₋₆ alkenyl)amino, C₂₋₆ alkynylamino, di(C₂₋₆ alkynyl)amino, alkenylamino, N—C₁₋₆ alkyl-N—C₂₋₆ alkynylamino and N—C₂₋₆ alkenyl-N—C₂₋₆ alkynylamino]; an optionally substituted amino [which may be substituted with one or two groups selected from C₁₋₆ alkyl, C₂₋₆ alkenyl, C₂₋₆ alkynyl, C₁₋₆ alkylsulfonyl, C₂₋₆ alkenylsulfonyl, C₂₋₆ alkynylsulfonyl, C₁₋₆ alkylcarbonyl, C₂₋₆ alkenylcarbonyl and C₂₋₆ alkynylcarbonyl]; C₁₋₆ alkylsulfonyl; C₂₋₆ alkenylsulfonyl; C₂₋₆ alkynylsulfonyl; C₁₋₆ alkylsulfonyl; C₂₋₆ alkenylsulfinyl; C₂₋₆ alkynylsulfinyl; formyl; optionally substituted C₃₋₈ cycloalkyl; an optionally substituted C₃₋₈ cycloalkenyl [where the cycloalkyl group and/or the cycloalkenyl group may independently and optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; a 5- to 14-membered non-aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; C₆₋₁₄ aromatic hydrocarbocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; and a 5- to 14-membered aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl].

In another embodiment, the invention provides compounds of Formula (V) wherein A¹, A² and A³ are each independently an optionally substituted C₃₋₈ cycloalkyl, an optionally substituted C₃₋₈ cycloalkenyl or an optionally substituted 5- to 14-membered non-aromatic hetero ring. In another embodiment, the invention provides the compound of Formula (V) wherein A¹, A² and A³ are each independently an optionally substituted C₆₋₁₄ aromatic hydrocarbon ring or an optionally substituted 5- to 14-membered aromatic hetero ring. In another embodiment, the invention provides the compound of Formula (V) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl or morpholyl; any of which may optionally have substituents. In another embodiment, the invention provides the compound of Formula (V) wherein A¹, A² and A³ are each independently selected from:

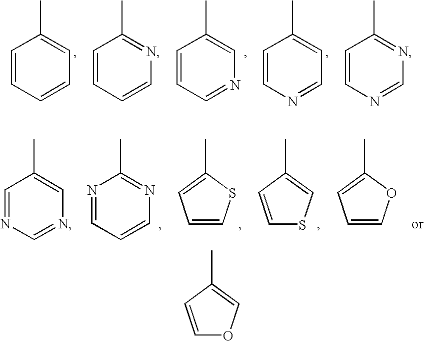

each of which may optionally be substituted. In another embodiment, the invention provides the compound of Formula (V) wherein A¹, A² and A³ are each independently substituted with hydroxyl, halogen, amino, or nitrile. In another embodiment, the invention provides the compound of Formula (V) wherein A¹, A² and A³ are each independently hydroxyl, halogen, amino, nitrile, or nitro. In another embodiment, the invention provides the compound of Formula (V) wherein Q is oxygen.

In another embodiment, the invention provides the compounds of Formula (V) wherein X¹, X² and X³ are each independently a single bond, —CH₂—, —CH(OH)—, —CH₂—CH₂—, —CH═CH—, —C≡C—, —O— or —CO—. In another embodiment, the invention provides the compounds of Formula (V) wherein X¹, X² and X³ are each a single bond. In another embodiment, the invention provides the compounds of Formula (V) wherein R¹⁷ and R¹⁸ are each independently hydrogen, fluorine, chlorine, bromine, iodine, methyl, ethyl, n-propyl, or iso-propyl. In another embodiment, the invention provides the compounds of Formula (V) wherein R¹⁷ and R¹⁸ are each hydrogen.

“Halogen atom” indicates fluorine, chlorine, bromine, iodine and the like, and the preferable halogen atoms include fluorine, chlorine and bromine.

“C₁₋₆ alkyl” refers to an alkyl group having 1 to 6 carbons, and examples include linear chain or branched chain alkyl groups such as methyl, ethyl, n-propyl, iso-propyl, n-butyl, iso-butyl, sec-butyl (1-methylpropyl), tert-butyl, iso-pentyl, n-pentyl, tert-pentyl (1,1-dimethylpropyl), 1,2-dimethylpropyl, 2,2-dimethylpropyl (neopentyl), 1-ethylpropyl, 2-methylbutyl, n-hexyl, iso-hexyl, 1,2-dimethylbutyl, 1-ethyl-1-methylpropyl, 1-ethyl-2-methylpropyl, 1,1,2-trimethylpropyl, 1,2,2-trimethylpropyl, 1-ethylbutyl, 1-methylbutyl, 1,1-dimethylbutyl, 2,2-dimethylbutyl, 1,3-dimethylbutyl, 2,3-dimethylbutyl, 3,3-dimethylbutyl, 2-ethylbutyl, 1-methylpentyl, 2-methylpentyl, 3-methylpentyl, and the like.

“C₂₋₆ alkenyl” refers to an alkenyl group having 2 to 6 carbons, and examples include vinyl, 1-ethylethenyl (1-buten-2-yl), allyl (2-propenyl), 1-propenyl, iso-propenyl, 2-methyl-1-propenyl, 1-methyl-1-propenyl (2-buten-2-yl), 2-methyl-2-propenyl, 1-methyl-2-propenyl, 1-butenyl (1-buten-1-yl), 2-butenyl (2-buten-1-yl), 3-butenyl, 1-pentenyl, 1-hexenyl, 1,3-hexadienyl, 1,6-hexadienyl, and the like.

“C₂₋₆ alkynyl” refers to an alkynyl group having 2 to 6 carbons, and examples include ethynyl, 1-propynyl, 2-propynyl, 1-butynyl, 2-butynyl, 3-butynyl, 1-ethyl-1-propynyl, 1-ethynyl-2-propynyl, 2-methyl-3-butenyl, 1-pentynyl, 1-hexynyl, 1,3-hexadiynyl, 1,6-hexadiynyl, and the like.

“C₁₋₆ alkoxy” refers to an alkoxy group having 1 to 6 carbons, and examples include methoxy, ethoxy, n-propoxy, iso-propoxy, n-butoxy, iso-butoxy, sec-butoxy, tert-butoxy, n-pentyloxy, iso-pentyloxy, tert-pentyloxy, 1,2-dimethylpropoxy, neopentyloxy, 1-ethylpropoxy, 1-methylbutoxy, 2-methylbutyoxy, n-hexyloxy, iso-hexyloxy, 1-ethyl-1-methylpropoxy, 1-ethyl-2-methylpropoxy, 1,1,2-trimethylpropoxy, 1,2,2-trimethylpropoxy, 1,1-dimethylbutoxy, 2,2-dimethylbutoxy, 2,3-dimethylbutoxy, 3,3-dimethylbutoxy, 2-ethylbutoxy, 1,3-dimethylbutoxy, 1-ethylbutoxy, 1-methylbutoxy, 1-methylpentyloxy, 2-methylpentyloxy, 3-methylpentyloxy, and the like.

“C₂₋₆ alkynyloxy” refers to an alkynyloxy group having 2 to 6 carbon atoms, and examples include ethynyloxy, 1-propynyloxy, 2-propynyloxy, 1-butynyloxy, 2-butynyloxy, 3-butynyloxy, 1-methyl-2-propynyloxy, 1-ethyl-2-propynyloxy, 1-ethynyl-2-propynyloxy, 1-pentynyloxy, 1-hexynyloxy, 1,3-hexadiynyloxy, 1,6-hexadiynyloxy, and the like.

“C₂₋₆ alkenyloxy” refers to an alkenyloxy group having 2 to 6 carbons, and examples include vinyloxy, 1-ethylethenyloxy (1-buten-2-yloxy), allyloxy (2-propenyloxy), 1-propenyloxy, iso-propenyloxy, 2-methyl-1-propenyloxy, 1-methyl-1-propenyloxy (2-buten-2-yloxy), 2-methyl-2-propenyloxy, 1-methyl-2-propenyloxy (1-buten-3-yloxy), 1-butenyloxy (1-buten-1-yloxy), 2-butenyloxy (2-buten-1-yloxy), 3-butenyloxy, 1-pentenyloxy, 1-hexenyloxy, 1,3-hexadienyloxy, 1,6-hexadienyloxy, and the like.

“C₃₋₈ cycloalkyl” refers to a cycloalkyl group composed of 3 to 8 carbon atoms, and examples include cyclopropyl, cyclobutyl, cyclopentyl, cyclohexyl, cycloheptyl, cyclooctyl, and the like.

“C₃₋₈ cycloalkenyl” refers to a cycloalkenyl group composed of 3 to 8 carbon atoms, and examples include cyclopropen-1-yl, 2-cyclopropen-1-yl, cyclobuten-1-yl, 2-cyclobuten-1-yl, 1,3-cyclobutadien-1-yl, cyclopenten-1-yl, 2-cyclopenten-1-yl, 3-cyclopenten-1-yl, 1,3-cyclopentadien-1-yl, 1,4-cyclopentadien-1-yl, 2,4-cyclopentadien-1-yl, cyclohexen-1-yl, 2-cyclohexen-1-yl, 3-cyclohexen-1-yl, 1,3-cyclohexadien-1-yl, 1,4-cyclohexadien-1-yl, 1,5-cyclohexadien-1-yl, 2,4-cyclohexadien-1-yl, 2,5-cyclohexadien-1-yl, cyclohepten-1-yl, 2-cyclohepten-1-yl, 3-cyclohepten-1-yl, 4-cyclohepten-1-yl, 1,3-cyclopentadien-1-yl, 1,4-cyclopentadien-1-yl, 1,5-cycloheptadien-1-yl, 1,6-cycloheptadien-1-yl, 2,4-cycloheptadien-1-yl, 2,5-cycloheptadien-1-yl, 2,6-cycloheptadien-1-yl, 3,5-cycloheptadien-1-yl, 1,3,5-cycloheptatrien-1-yl, 1,3,6-cycloheptatrien-1-yl, 1,4,6-cycloheptatrien-1-yl, 2,4,6-cycloheptatrien-1-yl, cycloocten-1-yl, 2-cycloocten-1-yl, 3-cycloocten-1-yl, 4-cycloocten-1-yl, 1,3-cyclooctadien-1-yl, 1,4-cyclooctadien-1-yl, 1,5-cyclooctadien-1-yl, 1,6-cyclooctadien-1-yl, 1,7-cyclooctadien-1-yl, 2,4-cyclooctadien-1-yl, 2,5-cyclooctadien-1-yl, 2,6-cyclooctadien-1-yl, 2,7-cyclooctadien-1-yl, 3,5-cyclooctadien-1-yl, 3,6-cyclooctadien-1-yl, 1,3,5-cyclooctatrien-1-yl, 1,3,6-cyclooctatrien-1-yl, 1,3,7-cyclooctatrien-1-yl, 1,4,6-cyclooctatrien-1-yl, 1,4,7-cyclooctatrien-1-yl, 1,5,7-cyclooctatrien-1-yl, 2,4,6-cyclooctatrien-1-yl, 2,4,7-cyclooctatrien-1-yl group, and the like.

“5- to 14-membered non-aromatic heterocyclic ring” refers to a mono-cyclic, di-cyclic, or tri-cyclic 5- to 14-membered non-aromatic heterocyclic ring which contains one or more hetero atoms selected from nitrogen, sulfur, and oxygen. Specific examples include pyrrolidinyl, pyrrolinyl, piperidyl, piperazinyl, pyrazolidinyl, imidazolidinyl, morpholinyl, tetrahydrofuryl, tetrahydropyranyl, dihydrofuryl, dihydropyranyl, imidazolinyl, oxazolinyl, and the like. Further, a group derived from a pyridone ring and a non-aromatic condensed ring (for example, a group derived from a phthalimide ring, a succinimide ring, and the like) are also included in the non-aromatic heterocyclic ring.

“C₆₋₁₄ aromatic hydrocarbocyclic ring and the aryl” refers to an aromatic hydrocarbocyclic ring which is composed of 6 to 14 carbon atoms, a mono-cyclic ring, and a condensed di-cyclic, tri-cyclic and the like. Specific examples include phenyl, indenyl, 1-naphthyl, 2-naphthyl, azulenyl, heptalenyl, biphenyl, indathenyl, acenaphthyl, fluorenyl, phenalenyl, phenanthrenyl, anthracenyl, cyclopentacyclooctenyl, benzocyclooctenyl and the like.

“5- to 14-membered aromatic heterocyclic ring and the heteroaryl ring” refers to mono-cyclic, di-cyclic, or tri-cyclic 5- to 14-membered aromatic heterocyclic ring which contain one or more hetero atoms selected from nitrogen, sulfur, and oxygen. Specific examples include (1) aromatic heterocyclic rings containing nitrogen such as pyrrolyl, pyridyl, pyridazinyl, pyrimidinyl, pyrazinyl, triazolyl, tetrazolyl, benzotriazolyl, pyrazolyl, imidazolyl, benzimidazolyl, indolyl, iso-indolyl, indolizinyl, prenyl, indazolyl, quinolyl, iso-quinolyl, quinoliziyl, phthalazyl, naphthylidinyl, quinoxalyl, quinazolinyl, cynnolinyl, pteridinyl, imidazotriazinyl, pyrazinopyridazinyl, acridinyl, phenanthridinyl, carbazolyl, carbazolinyl, perimidinyl, phenanthrolinyl, phenazinyl, imidazopyridyl, imidazopyrimidinyl, or pyrazolopyridyl; (2) aromatic heterocyclic rings containing sulfur such as thienyl or benzothienyl; (3) aromatic heterocyclic rings containing oxygen such as furyl, pyranyl, cyclopentapyranyl, benzofuryl, or iso-benzofuryl; and (4) aromatic heterocyclic rings containing 2 or more different hetero atoms such as thiazolyl, iso-thiazolyl, benzothiazolyl, benzothiadiazolyl, phenothiazinyl, isoxazolyl, furazanyl, phenoxazinyl, oxazolyl, isoxazoyl, benzoxazolyl, oxadiazolyl, pyrazoloxadiazolyl, imidazothiazolyl, thienofuranyl, furopyrrolyl or pyridoxadinyl.

The 1,2-dihydropyridine compound used in the methods and compositions described herein may be a compound of Formula (VI):

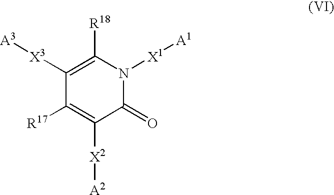

wherein X¹, X², X³, A¹, A², A³, R¹⁷ and R¹⁸ have the same meanings as defined in the above compound of Formula (V).

In another embodiment, the invention provides the compounds of Formula (VI) wherein A¹, A² and A³ are each independently an optionally substituted C₆₋₁₄ aromatic hydrocarbon ring or 5- to 14-membered aromatic hetero ring. In another embodiment, the invention provides the compounds of Formula (VI) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl, or morpholyl; wherein each may optionally be substituted. In another embodiment, the invention provides the compounds of Formula (VI) wherein A¹, A² and A³ are each independently selected from:

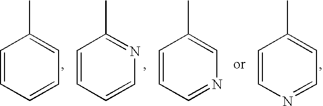

each of which may optionally be substituted. In another embodiment, the invention provides the compounds of Formula (VI) wherein the bonding site of the substituent at A¹, A² and A³ are in the α-position of the carbon atom bonding to the group X¹, X² and X³, respectively. In another embodiment, the invention provides the compounds of Formula (VI) wherein X¹, X² and X³ are single bonds. In another embodiment, the invention provides the compounds of Formula (VI) wherein R⁷ and R¹⁸ are hydrogen.

The 1,2-dihydropyridine compound used in the methods and compositions described herein may be Compound C:

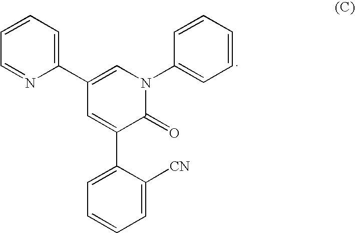

The IUPAC name for Compound C is 2-(2-oxo-1-phenyl-5-pyridin-2-yl-1,2-dihydropyridin-3-yl)benzonitrile. Compound C may also be referred to as 3-(2-cyanophenyl)-5-(2-pyridyl)-1-phenyl-1,2-dihydropyridin-2-one. Compound C is also known as perampanel.

The terms “Compound C,” “2-(2-oxo-1-phenyl-5-pyridin-2-yl-1,2-dihydropyridin-3-yl)benzonitrile,” “3-(2-cyanophenyl)-5-(2-pyridyl)-1-phenyl-1,2-dihydropyridin-2-one” and “perampanel” are intended to include pharmaceutically acceptable salts thereof, stereoisomers thereof, pharmaceutically acceptable salts of stereoisomers thereof, hydrates thereof, hydrates of pharmaceutically acceptable salts thereof, stereoisomers of hydrates thereof, and stereoisomer of hydrates of pharmaceutically acceptable salts thereof. In another embodiment, the terms “Compound C,” “2-(2-oxo-1-phenyl-5-pyridin-2-yl-1,2-dihydropyridin-3-yl)benzonitrile,” “3-(2-cyanophenyl)-5-(2-pyridyl)-1-phenyl-1,2-dihydropyridin-2-one” and “perampanel” are intended to include pharmaceutically acceptable salts thereof, hydrates thereof, and hydrates of pharmaceutically acceptable salts thereof.

In other embodiments, the 1,2-dihydropyridine compounds that are useful in the methods and compositions of the invention are 3-(2-cyanophenyl)-5-(2-methylsulfonylaminophenyl)-1-phenyl-1,2-dihydropyridin-2-one; 3-(2-chloro-3-pyridyl)-5-(2-pyridyl)-1-phenyl-1,2-dihydropyridin-2-one; 3-(2-cyanophenyl)-5-(2-pyridyl)-1-phenyl-1,2-dihydropyridin-2-one; 3-(2-cyanophenyl)-5-(2-pyridyl)-1-(3-nitrophenyl)-1,2-dihydropyridin-2-one; 3-(2-cyanophenyl)-5-(2-pyridyl)-1-(3-aminophenyl)-1,2-dihydropyridin-2-one; 3-(2-cyanophenyl)-5-(2-pyridyl)-1-(3-methylsulfonylaminophenyl)-1,2-dihydropyridin-2-one; 3-(2-cyanophenyl)-5-(2-pyridyl)-1-(3-methylaminophenyl)-1,2-dihydropyridin-2-one; 3-(2-cyanophenyl)-5-(2-pyridyl)-1-(3-dimethylaminophenyl)-1,2-dihydropyridin-2-one, 3-(2-cyanophenyl)-5-(2-pyridyl)-1-[3-(5-methoxymethyl-2-oxazolidinon-3-yl)-phenyl]-1,2-dihydropyridin-2-one; 3-(2-cyanophenyl)-5-(2-pyridyl)-1-(3-methoxycarbonylphenyl)-1,2-dihydropyridin-2-one; 3-(2-cyanophenyl)-5-(2-pyridyl)-1-(3-methylaminocarbonylphenyl)-1,2-dihydropyridin-2-one; 3-(2-cyano-3-pyridyl)-5-(2-pyridyl)-1-phenyl-1,2-dihydropyridin-2-one; 3-(2-chlorophenyl)-5-(2-pyridyl)-1-(4-hydroxyphenyl)-1,2-dihydropyridin-2-one; 3-(2-chlorophenyl)-5-(2-pyridyl)-1-(4-dimethylaminoethoxyphenyl)-1,2-dihydropyridin-2-one; 3-(2-cyanophenyl)-5-(2-pyridyl)-1-(3-formylphenyl)-1,2-dihydropyridin-2-one; 3-(2-cyanophenyl)-5-(2-pyridyl)-1-(3-hydroxymethylphenyl)-1,2-dihydropyridin-2-one; 3-(2-cyanophenyl)-5-(2-pyridyl)-1-(3-cyanomethylphenyl)-1,2-dihydropyridine-2-one; 3-(2-cyanophenyl)-5-(2-pyridyl)-1-(3-acetylaminomethylphenyl)-1,2-dihydropyridin-2-one; 3-(2-cyanophenyl)-5-(2-pyridyl)-1-(3-methylsulfonylaminomethylphenyl)-1,2-dihydropyridin-2-one; 3-(2-cyanophenyl)-5-(2-pyridyl)-1-(3-acetoxymethylphenyl)-1,2-dihydropyridin-2-one; 3-(2-cyanophenyl)-5-(2-pyridyl)-1-(4-methylthiophenyl)-1,2-dihydropyridin-2-one; 3-(2-cyanophenyl)-5-(2-pyridyl)-1-(4-methylsulfonylpheny-1)-1,2-dihydropyridin-2-one; 3-(2-cyanophenyl)-5-(2-formylthiophen-3-yl)-1-phenyl-1,2-dihydropyridin-2-one; 3-(2-cyanophenyl)-5-(2-diethylaminomethylthiophen-3-yl)-1-phenyl-1,2-dihydropyridin-2-one; 3-(2-cyanophenyl)-5-(2-hydroxymethylthiophen-3-yl)-1-phenyl-1,2-dihydropyridine-2-one; 3-(2-cyanophenyl)-5-(2-pyridyl)-1-benzyl-1,2-dihydropyridin-2-one; 3-(2-cyanophenyl)-5-(2-pyridyl)-1-(3-pyridyl)-1,2-dihydropyridin-2-one; 3-(2-cyanophenyl)-5-phenyl-(2-pyridyl)-1,2-dihydropyridin-2-one, 3-(2-cyanophenyl)-1,5-diphenyl-1,2-dihydropyridin-2-one; 3-(2-cyanophenyl)-5-(2-methoxyphenyl)-1-phenyl-1,2-dihydropyridin-2-one; 3-(2-cyanophenyl)-5-(3,4-dimethoxyphenyl)-1-phenyl-1,2-dihydropyridin-2-one; 3-(2-cyanophenyl)-5-(thiophen-3-yl)-1-phenyl-1,2-dihydropyridin-2-one; 3-(2-cyanophenyl)-5-(2-fluorophenyl)-1-phenyl-1,2-dihydropyridin-2-one; 3-(2-cyanophenyl)-5-(thiophen-2-yl)-1-phenyl-1,2-dihydropyridin-2-one, 3-(2-cyanophenyl)-5-(3-furfuryl)-1-phenyl-1,2-dihydropyridin-2-one; 3-(2-cyanophenyl)-5-(2-furfuryl)-1-phenyl-1,2-dihydropyridin-2-one; 3-(2-chlorophenyl)-5-(2-pyridyl)-1-phenyl-1,2-dihydropyridin-2-one; 3-(2-methoxycarbonylphenyl)-5-(2-pyridyl)-1-phenyl-1,2-dihydropyridin-2-one; 3-phenyl-5-(2-pyridyl)-1-phenyl-1,2-dihydropyridin-2-one; 3-(2-fluorophenyl)-5-(2-pyridyl)-1-phenyl-1,2-dihydropyridin-2-one; 3-(2-chlorophenyl)-5-(2-pyridyl)-1-(3-methoxyphenyl)-1,2-dihydropyridin-2-one; 3-(2-fluoro-3-pyridyl)-5-(2-pyridyl)-1-phenyl-1,2-dihydropyridin-2-one; 3-(4-methoxy-3-pyridyl)-5-(2-pyridyl)-1-phenyl-1,2-dihydropyridin-2-one; 3-(2-fluoro-3-pyridyl)-5-(2-pyridyl)-1-(3-methoxyphenyl)-1,2-dihydropyridin-2-one; 3-(2-fluoro-3-pyridyl)-5-(2-pyridyl)-1-(3-fluorophenyl)-1,2-dihydropyridin-2-one; 3-(2-cyanophenyl)-5-(2-pyridyl)-1-(4-fluorophenyl)-1,2-dihydropyridin-2-one; 3-(2-cyanophenyl)-5-(2-pyridyl)-1-(3-fluorophenyl)-1,2-dihydropyridin-2-one; 3-(2-cyanophenyl)-5-(2-pyridyl)-1-(4-methoxyphenyl)-1,2-dihydropyridin-2-one; 3-(2-cyanophenyl)-5-(2-pyridyl)-1-(3-methoxy-phenyl)-1,2-dihydropyridin-2-one; 3-phenyl-5-(2-pyridyl)-1-(3-fluorophenyl-)-1,2-dihydropyridin-2-one; 3-(2-chlorophenyl)-5-(2-pyridyl)-1-(4-fluorophenyl)-1,2-dihydropyridin-2-one; 3-(2-cyanophenyl)-5-(2-pyridyl)-1-(4-formylphenyl)-1,2-dihydropyridin-2-one; 3-(2-cyanophenyl)-5-(2-pyridyl)-1-(2-formylphenyl)-1,2-dihydropyridin-2-one; 3-(2-cyanophenyl)-5-(2-pyridyl)-1-(3-chlorophenyl)-1,2-dihydropyridin-2-one; 3-(2-cyanophenyl)-5-(2-pyridyl)-1-(3-tolyl)-1,2-dihydropyridin-2-one; 3-(2-cyanophenyl)-5-(2-pyridyl)-1-(3-trifluoromethylphenyl)-1,2-dihydropyridin-2-one; 3-(2-cyanophenyl)-5-(2-pyridyl)-1-(thiophen-3-yl)-1,2-dihydropyridin-2-one; 3-(2-cyanophenyl)-5-(2-pyridyl)-1-(3-furfuryl)-1,2-dihydropyridin-2-one; 3-(2-cyanophenyl)-5-(2-pyridyl)-1-(4-tolyl)-1,2-dihydropyridin-2-one; 3-(2-cyanophenyl)-5-(2-pyridyl)-1-(4-trifluoromethylphenyl)-1,2-dihydropyridin-2-one; 3-(2-cyanophenyl)-5-(2-pyridyl)-1-(2-methoxypyridin-5-yl)-1,2-dihydropyridin-2-one; 3-(2-cyanophenyl)-5-(2-pyridyl)-1-(pyrimidin-5-yl)-1,2-dihydrop-yridin-2-one; 3-(2-cyanophenyl)-5-(2-pyridyl)-1-(3-benzyloxymethylpyridin-5-yl)-1,2-dihydropyridin-2-one; 3-(2-cyanophenyl)-5-(2-pyridyl)-1-(2-ethylthiopyridin-5-yl)-1,2-dihydropyridin-2-one; 3-(2-cyanophenyl)-5-(2-pyridyl-)-1-(4-pyridyl)-1,2-dihydropyridin-2-one; 3-(2-cyanophenyl)-5-(2-pyridyl)-1-(3-methoxypyridin-5-yl)-1,2-dihydropyridin-2-one; 3-(2-cyanophenyl)-5-(2-pyridyl)-1-(2-chloropyridin-5-yl)-1,2-dihydropyridin-2-one; 3-(2-cyanophenyl)-5-(2-pyridyl)-1-(2-fluoropyridin-5-yl)-1,2-dihydropyridin-2-one; 3-(2-cyanophenyl)-5-(2-pyridyl)-1-(2-methoxyphenyl)-1,2-dihydropyridin-2-one; 3-phenyl-5-(2-pyridyl)-1-(3-pyridyl)-1,2-dihydropyridin-2-one; 3-(2-chlorophenyl)-5-(2-pyridyl)-1-(3-pyridyl)-1,2-dihydropyridin-2-one; 3-(thiophen-3-yl)-5-(2-pyridyl)-1-(3-pyridyl)-1,2-dihydropyridin-2-one; 3-(2,6-dimethylphenyl)-5-(2-pyridyl)-1-(3-pyridyl)-1,2-dihydropyridin-2-one; 3-(2-cyanothiophen-3-yl)-5-(2-pyridyl)-1-(3-pyridyl)-1,2-dihydropyridin-2-one; 3-(2-fluoro-3-pyridyl)-5-(2-pyridyl)-1-(3-pyridyl)-1,2-dihydropyridin-2-one; 3-(2-chlorophenyl)-5-(2-pyridyl)-1-(3-hydroxyphenyl)-1-,2-dihydropyridin-2-one; 3-(2-chlorophenyl)-5-(2-pyridyl)-1-(3-dimethylaminoethoxyphenyl)-1,2-dihydropyridin-2-one; 342-chlorophenyl)-5-(2-pyridyl)-1-(3-dimethylaminopropoxyphenyl)-1,2-dihydropyridin-2-one; 3-(2-cyanophenyl)-5-(2-pyridyl)-1-(2-hydroxymethylphenyl)-1,2-dihydropyridin-2-one; 3-(2-cyanophenyl)-5-(2-pyridyl)-1-(4-cyanomethylphenyl)-1,2-dihydropyridin-2-one; 3-(2-cyanophenyl)-5-(2-pyridyl)-1-(2-cyanomethylphenyl)-1,2-dihydropyridin-2-one; 3-(2-cyanophenyl)-5-(6-diethylaminomethyl-2-pyridyl)-1-phenyl-1,2-dihydropyridin-2-one; 3-(2-cyanophenyl)-1-phenyl-5-(2-pyrimidinyl)-1,2-dihydropyridin-2-one; 3-(2-hydroxypyridin-6-yl)-1-phenyl-5-(2-pyridyl)-1,2-dihydropyridin-2-one; 1-(2-aminobenzothiazol-6-yl)-3-(2-cyanophenyl)-5-(2-pyridyl)-1,2-dihydropyridin-2-one; 3-(2-cyanophenyl)-5-(2-pyridyl)-1-(1-benzyl-1,2,3,6-tetrahydropyridin-5-yl)-1,2-dihydropyridin-2-one; 3-[2-(5-methyl-1,2,4-oxadiazol-3-yl)phenyl]-1-phenyl-5-(2-pyridyl)-1,2-dihydropyridin-2-one; 3-(2-cyanophenyl)-5-(6-methylpyridin-2-yl)-1-phenyl-1,2-dihydropyridin-2-one; 3-(2-cyanophenyl)-5-(5-methylpyridin-2-yl)-1-phenyl-1,2-dihydropyridin-2-one; 3-(2-cyanophenyl)-5-(3-hydroxypyridin-2-yl)-1-phenyl-1,2-dihydropyridin-2-one; 3-(2-cyanophenyl)-1-phenyl-5-(2-thiazolyl)-1,2-dihydropyridin-2-one; 3-(2-cyanophenyl)-5-(2-methoxypyridin-6-yl)-1-phenyl-1,2-dihydropyridin-2-one; 1-(4-aminophenyl)-3-(2-cyanophenyl)-5-(2-pyridyl)-1,2-dihydropyridin-2-one; 1-(3-aminophenyl)-3-(2-cyanophenyl)-5-(2-pyrimidinyl)-1,2-dihydropyridin-2-one; 3-(2-cyanophenyl)-5-(2-pyridyl)-1-(2-aminotoluen-4-yl)-1-, 2-dihydropyridin-2-one; 3-(2-cyanophenyl)-1-[3-(dimethylaminoethoxy)phenyl]-5-(2-pyridyl)-1,2-dihydropyridin-2-one; 3-(2-cyanophenyl)-1-[3-(piperidinoethoxy)phenyl]-5-(2-pyridyl)-1,2-dihydropyridin-2-one; 3-(2-cyanophenyl)-1-[3-(pyrrolidinoethoxy)phenyl]-5-(2-pyridyl)-1,2-dihyd-ropyridin-2-one; 3-(2-cyanophenyl)-1-[3-(diisopropylaminoethoxy)phenyl]-5-(2-pyridyl)-1,2-dihydropyridin-2-one; 3-(2-cyanophenyl)-1-[3-(4-piperidinobutyl-1-oxy)phenyl]-5-(2-pyridyl)-1,2-dihydropyridin-2-one; 3-(2-cyanophenyl)-1-(4-nitrophenyl)-5-(2-pyridyl)-1,2-dihydropyridin-2-one; 1-phenyl-5-(2-pyridyl)-3-(2-thiazolyl)-1,2-dihydropyridin-2-one; 3-(2-cyanophenyl)-1-(3-pyridyl)-5-(2-pyrimidinyl)-1,2-dihydropyridin-2-one; 3-(2-fluoropyridin-3-yl)-1-phenyl-5-(2-pyrimidinyl)-1,2-dihydropyridin-2-one; 3-(2-cyanopyridin-3-yl)-1-phenyl-5-(2-pyrimidinyl)-1,2-dihydropyridin-2-one; 3-(2-cyanophenyl)-1-(3-nitrophenyl)-5-(2-pyrimidinyl)-1,2-dihydropyridin-2-one; 3-(2-nitrophenyl)-1-phenyl-5-(2-pyridyl)-1,2-dihydropyridin-2-one; 3-(2-formylthiophen-3-yl)-5-(2-pyridyl)-1-phenyl-1,2-dihydropyrid-in-2-one; 3-(2-cyanophenyl)-5-(2-pyridyl)-1-(2-naphthyl)-1,2-dihydropyridin-2-one; 3-(2-cyanophenyl)-5-(2-pyridyl)-1-(1-naphthyl)-1,2-dihydropyridin-2-one; 5-(2-aminopyridin-6-yl)-3-(2-cyanophenyl)-1-phenyl-1,2-dihydropyridin-2-one; 5-(2-bromopyridin-6-yl)-3-(2-cyanophenyl)-1-phenyl-1,2-dihydropyridin-2-one; 3-(2-cyanophenyl)-5-(2-morphorinopyridin-6-yl)-1-phenyl-1,2-dihydropyridin-2-one; 3-(2-cyanophenyl)-1-(3-hydroxyphenyl)-5-(2-pyridyl)-1,2-dihydropyridin-2-one; 3-(2-cyanophenyl)-1-[3-(4-piperidyloxy)]phenyl-5-(2-pyridyl)-1,2-dihydropyridin-2-one; 1-[3-(N-acetylpiperidin-4-yl-oxy)phenyl]-3-(2-cyanophenyl)-5-(2-pyridyl)-1,2-dihydropyridin-2-one; 3-(2-cyanophenyl)-1-{3-[1-(methanesulfonyl)piperidin-4-yl-oxy]phenyl}-5-(2-pyridyl)-1,2-dihydropyridin-2-one; 1-[3-(N-methylpiperidin-4-yl-oxy)pheny-l]-3-(2-cyanophenyl)-5-(2-pyridyl)-1,2-dihydropyridin-2-one; 3-(6-chloro-1H-benzimidazol-2-yl)-5-(2-pyridyl)-1-phenyl-1,2-dihydropyridin-2-one; 3-(2-cyanophenyl)-5-(2-pyridyl)-1-(2-nitrotoluen-4-yl)-1,2-dihydropyridin-2-one; 3-(2-cyanothiophen-3-yl)-5-(2-pyridyl)-1-phenyl-1,2-dihydropyridin-2-one; 3-[2-(5-oxazolyl)phenyl]-1-phenyl-5-(2-pyridyl)-1,2-dihydropyridin-2-one; 3-[2-(5-oxazolyl)thiophen-3-yl]-1-phenyl-5-(2-pyridyl)-1,2-dihydropyridin-2-one; and 3-(2-ethoxycarbonylvinylthiophen-3-yl)-5-(2-pyridyl)-1-phenyl-1,2-dihydropyridin-2-one.

The 1,2-dihydropyridine compounds and methods for making the 1,2-dihydropyridine compounds are described in U.S. Pat. No. 6,949,571, US Publication No. 2004/0023973, and PCT Publication Nos. WO 03/047577, WO 04/009553, WO 06/004100, WO 06/004107, WO 07/072,868, and WO 07/072,869, the disclosures of each of which are incorporated by reference herein in their entirety.

Methods for administering, dosing, and making other AMPA receptor antagonists such as quinoxalinedione aminoalkylphosphonates are described, for example, in WO 2005/094797 and WO 98/17672. In one embodiment, the AMPA receptor antagonist is a compound of Formula (VII):

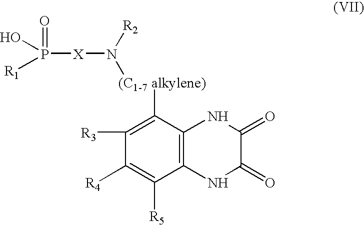

wherein R₁ is hydroxy, an aliphatic group, an aryl aliphatic group, or an aromatic group; R₂ is hydrogen, an aliphatic group, or an aryl aliphatic group; R₃, R₄ and R₅ are each independently hydrogen, halogen, cyano, nitro, trifluoromethyl, or a C₁₋₇ alkyl group; and X is an aliphatic group, a cycloaliphatic group, an aryl aliphatic group, a heteroaryl aliphatic group, or an aromatic group.

In other embodiments, the AMPA receptor antagonist may be becampanel, EGIS 8332 (7-acetyl-5-(4-aminophenyl)-8,9-dihydro-8-methyl-7H-1,3-dioxolo[4,5-h][2,3]benzodiazepine-8-carbonitrile); GYKI 47261 (4-(7-chloro-2-methyl-4H-3,10,10a-triaza-benzo[f]azulen-9-yl)phenylamine)); irampanel (N,N-dimethyl-2-[2-(3-phenyl-1,2,4-oxadiazol-5-yl)phenoxy]ethanamine); KRP 199 ((7-[4-[[[[4-carboxyphenyl)-amino]carbonyl]oxy]methyl]-1H-imidazol-1-yl]-3,4-dihydro-3-oxo-6-(trifluoromethyl)-2-quinoxalinecarboxylic acid); NS 1209 (2-[[[5-[4-[(dimethylamino)-sulfonyl]phenyl]-1,2,6,7,8,9-hexahydro-8-methyl-2-oxo-3H-pyrrolo[3,2-h]isoquinolin-3-ylidene]amino]oxy]-4-hydroxybutanoic acid monosodium salt; topiramate (TOPAMAX®); talampanel (LY-300164, (R)-7-acetyl-5-(4-aminophenyl)-8,9-dihydro-8 methyl-7H-1,3-dioxolo[4,5-h][2,3]benzo-diazepine; YM9OK (6-imidazol-1-yl-7-nitro-1,4-dihydro-quinoxaline-2,3-dione); S-34730 (7-chloro-6-sulfamoyl-2-(1H)-quinolinone-3-phosphonic acid); Zonampanel (YM-872; (7-imidazol-1-yl-6-nitro-2,3-dioxo-3,4-dihydro-2H-quinoxalin-1-yl)-acetic acid); GYKI 52466 (4-(8-methyl-9H-1,3-dioxa-6,7-diaza-cyclohepta[f]inden-5-yl)-phenylamine); ZK 200775 (MPQX, (7-morpholin-4-yl-2,3-dioxo-6-trifluoromethyl-3,4-dihydro-2H-quinoxalin-1-ylmethyl)-phosphonic acid); CP-465022 (3-(2-chlorophenyl)-2-[2-(6-diethylaminomethyl-pyridin-2-yl)-vinyl]-6-fluoro-3H-quinazolin-4-one); SYM-2189 (4-(4-amino-phenyl)-6-methoxy-1-methyl-1H-phthalazine-2-carboxylic acid propylamide); SYM-2206 (8-(4-amino-phenyl)-5-methyl-5H-[1,3]dioxolo[4,5-g]phthalazine-6-carboxylic acid propylamide); RPR-117824 ((4-oxo-2-phosphono-5,10-dihydro-4H-imidazo[1,2-a]indeno[1,2-e]pyrazin-9-yl)-acetic acid); or LY-293558 (6-[2-(1H-tetrazol-5-yl)-ethyl]-decahydro-isoquinoline-3-carboxylic acid).

The NMDA receptor antagonist, which is an active ingredient, may be any known in the art. In one embodiment, the NMDA receptor antagonist is represented by Formula (VIII), a pharmaceutically acceptable salt thereof, a stereoisomer thereof, or a pharmaceutically acceptable salt of a stereoisomer thereof:

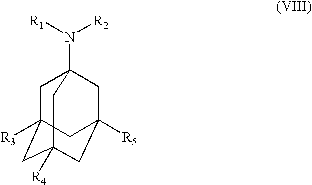

wherein R₁ and R₂ are each independently hydrogen or a straight or branched chain C₁₋₆ alkyl group, or R₁ and R₂ in conjunction with the nitrogen atom form a C₅₋₆ heterocyclic group; R₃ and R₄ are each independently hydrogen, a straight or branched chain C₁₋₆ alkyl group, a C₅₋₆ cycloalkyl group, or phenyl; and R₅ is hydrogen or a straight or branched chain C₁₋₆ alkyl group. In one embodiment, R₁, R₂, R₃, R₄ and R₅ are not simultaneously hydrogen atoms. In one embodiment, the pharmaceutically acceptable salt is a pharmaceutically acceptable acid addition salt. Exemplary branched or straight chain C₁₋₆ alkyl groups include methyl, ethyl, iso-propyl, n-propyl, n-butyl, iso-butyl, t-butyl, n-pentyl, n-hexyl, and isomers thereof.

In one embodiment, the compound of Formula (VIII) is Compound (D), a pharmaceutically acceptable salt thereof, a stereoisomer thereof, or a pharmaceutically acceptable salt of a stereoisomer thereof:

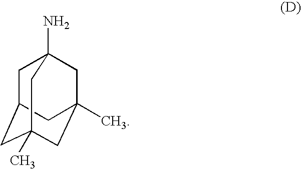

In another embodiment, the compound of Formula (VIII) is Compound (D1) or a pharmaceutically acceptable salt thereof:

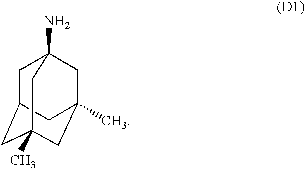

Compound (D1) is also known as memantine and can be in the form of any pharmaceutically acceptable salt known in the art. In one embodiment, memantine is in the form of a hydrochloride salt. Compounds (D) and (D1) are also known as 1-amino-3,5-dimethyl-adamantane.

In other embodiments, the adamantane compound is 1-amino adamantine; 1-amino-3-phenyl adamantine; 1-amino-methyl-adamantane; 1-amino-3,5-dimethyl adamantine; 1-amino-3-ethyl adamantine; 1-amino-3-isopropyl adamantine; 1-amino-3-n-butyl adamantine; 1-amino-3,5-diethyl adamantine; 1-amino-3,5-diisopropyl adamantine; 1-amino-3,5-di-n-butyl adamantine; 1-amino-3-methyl-5-ethyl adamantine; 1-N-methylamino-3,5-dimethyl adamantine; 1-N-ethylamino-3,5-dimethyl adamantine; 1-N-isopropyl-amino-3,5-dimethyl adamantine; 1-N,N-dimethyl-amino-3,5-dimethyl adamantine; 1-N-methyl-N-isopropyl-amino-3-methyl-5-ethyl adamantine; 1-amino-3-butyl-5-phenyl adamantine; 1-amino-3-pentyl adamantine; 1-amino-3,5-dipentyl adamantine; 1-amino-3-pentyl-5-hexyl adamantine; 1-amino-3-pentyl-5-cyclohexyl adamantine; 1-amino-3-pentyl-5-phenyl adamantine; 1-amino-3-hexyl adamantine; 1-amino-3,5-dihexyl adamantine; 1-amino-3-hexyl-5-cyclohexyl adamantine; 1-amino-3-hexyl-5-phenyl adamantine; 1-amino-3-cyclohexyl adamantine; 1-amino-3,5-dicyclohexyl adamantine; 1-amino-3-cyclohexyl-5-phenyl adamantine; 1-amino-3,5-diphenyl adamantine; 1-amino-3,5,7-trimethyl adamantine; 1-amino-3,5-dimethyl-7-ethyl adamantine; 1-amino-3,5-diethyl-7-methyl adamantine; 1-N-pyrrolidino and 1-N-piperidine derivatives; 1-amino-3-methyl-5-propyl adamantine; 1-amino-3-methyl-5-butyl adamantine; 1-amino-3-methyl-5-pentyl adamantine; 1-amino-3-methyl-5-hexyl adamantine; 1-amino-3-methyl-5-cyclohexyl adamantine; 1-amino-3-methyl-5-phenyl adamantine; 1-amino-3-ethyl-5-propyl adamantine; 1-amino-3-ethyl-5-butyl adamantine; 1-amino-3-ethyl-5-pentyl adamantine; 1-amino-3-ethyl-5-hexyl adamantine; 1-amino-3-ethyl-5-cyclohexyl adamantine; 1-amino-3-ethyl-5-phenyl adamantine; 1-amino-3-propyl-5-butyl adamantine; 1-amino-3-propyl-5-pentyl adamantine; 1-amino-3-propyl-5-hexyl adamantine; 1-amino-3-propyl-5-cyclohexyl adamantine; 1-amino-3-propyl-5-phenyl adamantine; 1-amino-3-butyl-5-pentyl adamantine; 1-amino-3-butyl-5-hexyl adamantine; 1-amino-3-butyl-5-cyclohexyl adamantine; their N-methyl, N,N-dimethyl, N-ethyl, N-propyl derivatives. Each of these compounds can be in the form of a pharmaceutically acceptable salt thereof, a stereoisomer thereof, or a pharmaceutically acceptable salt of a stereoisomer thereof.

Methods for preparing Compounds (VIII), (D) and (D1) are described in U.S. Pat. No. 5,061,703, the disclosure of which is incorporated by reference herein in its entirety. Memantine is commercially available as NAMENDA® from Forest Laboratories.

In another embodiment, the NMDA receptor antagonist is a compound of Formula (IX), a pharmaceutically acceptable salt thereof, a stereoisomer thereof, or a pharmaceutically acceptable salt of a stereoisomer thereof:

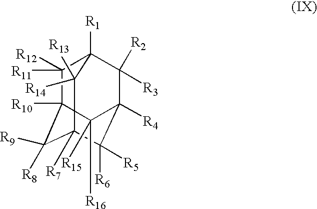

wherein R₁ is an amino group or

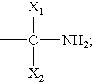

where X₁ and X₂ are each independently hydrogen or a C₁₋₅ aliphatic group; R₂ through R₁₆ are each independently hydrogen or a chain C₁₋₅ aliphatic group. R₄ and R₁₀ may alternatively and independently be halogen or an acyl group.

In one embodiment, the compound of Formula (IX) is amantadine, a pharmaceutically acceptable salt thereof, a stereoisomer thereof, or a pharmaceutically acceptable salt of a stereoisomer thereof; wherein R₂-R₁₆ are all hydrogen atoms. In another embodiment, the compound of Formula (IX) is memantine; a pharmaceutically acceptable salt thereof, a stereoisomer thereof, or a pharmaceutically acceptable salt of a stereoisomer thereof; memantine is shown above as Compounds (D) and (D1). In another embodiment, the compound of Formula (IX) is rimantadine, a pharmaceutically acceptable salt thereof, a stereoisomer thereof, or a pharmaceutically acceptable salt of a stereoisomer thereof; wherein R₂-R₁₇ are all hydrogen atoms and R₁ is

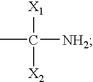

wherein X₁ is hydrogen and X₂ is methyl.

Methods for preparing Compound (IX) are described in U.S. Pat. No. 5,614,560, the disclosure of which is incorporated by reference herein in its entirety.

In another embodiment, the NMDA receptor antagonist is neramexane (i.e., 1-amino-1,3,3,5,5-pentamethylcyclohexane) or a pharmaceutically acceptable salt thereof as described in U.S. Pat. No. 6,034,134 and WO 2004/037234, the disclosures of which are incorporated by reference herein in their entirety.

In other embodiments, exemplary NMDA receptor antagonists that can be used in the compositions, combinations, and methods described herein, include ketamine or a pharmaceutically acceptable salt thereof, eliprodil or a pharmaceutically acceptable salt thereof, ifenprodil or a pharmaceutically acceptable salt thereof, dizocilpine or a pharmaceutically acceptable salt thereof, remacemide or a pharmaceutically acceptable salt thereof, iamotrigine or a pharmaceutically acceptable salt thereof, riluzole or a pharmaceutically acceptable salt thereof, aptiganel or a pharmaceutically acceptable salt thereof, phencyclidine or a pharmaceutically acceptable salt thereof, flupirtine or a pharmaceutically acceptable salt thereof, celfotel or a pharmaceutically acceptable salt thereof, felbamate or a pharmaceutically acceptable salt thereof, spermine or a pharmaceutically acceptable salt thereof, spermidine or a pharmaceutically acceptable salt thereof, levemopamil or a pharmaceutically acceptable salt thereof, dextromethorphan ((+)-3-hydroxy-N-methylmorphinan) or a pharmaceutically acceptable salt thereof, dextrorphan ((+)-3-hydroxy-N-methylmorphinan) or a pharmaceutically acceptable salt thereof, and the like. These NMDA receptor antagonists are known in the art and described, for example, in PCT Publication Nos. WO 2004/071431 and WO 2006/121560, the disclosures of which are incorporated by reference herein in their entirety.

Other NMDA receptor antagonists known in the art can be used in the compositions, combinations and methods described herein, including the NMDA receptor antagonists described in U.S. Pat. Nos. 4,346,112; 5,061,703; 5,334,618; 5,382,601; 6,444,702; 6,620,845; and 6,662,845, the disclosures of which are incorporated by reference herein in their entirety.

The dosage form of the formulation included in the combination, kit and/or pharmaceutical composition of the invention is not particularly limited. The combination, kit and/or pharmaceutical composition of the invention is useful as a combination, kit and/or a pharmaceutical composition for treating dementia and/or mild cognitive impairments; for the prophylaxis of dementia and/or mild cognitive impairments; and for delaying the onset of dementia and/or mild cognitive impairments.

The combination, kit and/or pharmaceutical composition of the invention may be used as a drug for treating dementia and/or mild cognitive impairments; for the prophylaxis of dementia and/or mild cognitive impairments; and for delaying the onset of dementia and/or mild cognitive impairments.

The combination, kit and/or pharmaceutical composition of the invention may be administered to a patient.

The combination, kit and/or pharmaceutical composition of the invention may be used through oral or parental administration. When the combination, kit and/or pharmaceutical composition of the invention is used, the given dose of the compound of the invention differs depending on the degree of the symptom, age, sex, weight and sensitivity difference of the patient, administration mode, administration period, administration interval, nature, prescription and the type of the pharmaceutical formulation, and the type of the active element.

The pharmaceutical composition of the invention may be made into various forms, for example, into solid oral formulations, injectable solution or the like.

The active ingredients described herein can be administered orally, parenterally, or rectally in dosage unit formulations containing conventional nontoxic pharmaceutically acceptable excipients as desired. The term parenteral includes subcutaneous, intravenous, intramuscular, intrathecal, intrasternal injection, or infusion techniques.

For oral administration, the daily dose of the cinnamide compounds of the invention may be from 30 μg/day to 10 g/day; from 100 pg/day to 5 g/day; or from 100 μg/day to 1 g/day. For parenteral administration, the daily dose of the cinnamide compounds may be from 30 μg/day to 3 g/day; from 100 μg/day to 1 g/day; or from 100 μg/day to 500 mg/day. The cinnamide compounds may be administered as a single dose or in multiple doses.

The daily dose of the cholinesterase inhibitors is usually from 0.01 mg/day to 300 mg/day; from 0.1 mg/day to 50 mg/day; or from 1 mg/day to 25 mg/day. Donepezil hydrochloride, commercially available as ARICEPT® (Eisai Inc., Teaneck, N.J.), can be administered as tablets containing any one of 3 mg donepezil hydrochloride, 5 mg donepezil hydrochloride or 10 mg donepezil hydrochloride. The tablets can be administered one to about four times a day.

The daily dose of the AMPA receptor antagonists, such as the 1,2-dihydropyridine compounds of the invention (e.g., 3-(2-cyanophenyl)-5-(2-pyridyl)-1-phenyl-1,2-dihydropyridin-2-one) is usually 30 μg/day to 10 g/day; from 100 μg/day to 5 g/day or, from 100 μg/day to 100 mg/day in the case of oral administration. For administration by injection, the daily dose is usually 30 μg/day to 1 g/day; from 100 μg/day to 500 mg/day or, from 100 μg/day to 30 mg/day. The AMPA receptor antagonists are administered once daily or in several portions a day.

The daily dose of the NMDA receptor antagonists, such as the adamantane compounds of the invention (e.g., memantine) is usually 0.01 mg/day to 100 mg/day; from 0.01 mg/day to 1000 mg/day; from 0.1 mg/day to 500 mg/day; from 1 mg/day to 100 mg/day; from 1 mg/day to 50 mg/day; or from 10 mg/day to 30 mg/day. The NMDA receptor antagonists are administered once daily or in several portions a day.

The other active ingredients described herein are commercially available or can be prepared by processes known in the art and can be administered in dosages known in the art.

In one embodiment, the mode of administration is by injection, such as subcutaneous injection, intramuscular injection, intravenous injection, or intra-arterial injection. Injectable preparations, for example, sterile injectable aqueous or oleaginous suspensions can be formulated according to the art using suitable dispersing or wetting agents, suspending agents (e.g., methylcellulose, Polysorbate 80, hydroxyethylcellulose, acacia, powdered tragacanth, sodium carboxymethylcellulose, polyoxyethylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like), stabilizers (e.g., sodium sulfite and sodium metasulfite; examples of the preservative include methyl parahydroxybenzoate, ethyl parahydroxybenzoate, sorbic acid, phenol, cresol, chlorocresol, and the like), tonicity agents and preservatives. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids, such as oleic acid, can be used in the preparation of injectables. The preparations can be lyophilized by methods known in the art.

In order to prepare a solid oral formulation, an excipient, and if necessary, a binder, disintegrant, lubricant, colorant, a flavoring agent and the like are added to the principal agent, and then made into a tablet, a coated tablet, granule, fine granule, dispersant, a capsule or the like according to a conventional method.

For example, lactose, cornstarch, sucrose, glucose, sorbit, crystalline cellulose, silicon dioxide or the like may be used as the excipient; for example, polyvinyl alcohol, ethyl cellulose, methyl cellulose, gum arabic, hydroxypropyl cellulose, hydroxypropylmethyl cellulose or the like may be used as the binder; for example, magnesium stearate, talc, silica or the like may be used as the lubricant; those that are allowed to be added to drugs may be used as the colorant; and for example, cocoa powder, menthol, aromatic acid, peppermint oil, camphor, cinnamon powder or the like may be used as the flavoring agent. Of course, if necessary, these tablets and granule may be coated appropriately with sugar coating, gelatin coating or else.

Solid dosage forms for oral administration can include chewing gum, capsules, tablets, sublingual tablets, powders, granules, and gels. In such solid dosage forms, the active ingredient(s) can be admixed with one or more inert diluents such as lactose or starch. As is normal practice, such dosage forms can also comprise other substances including lubricating agents such as magnesium stearate. In the case of capsules, tablets, and pills, the dosage forms can also comprise buffering agents. The tablets can be prepared with enteric or film coatings.

To make tablets, the active ingredient(s) can be admixed with pharmaceutically acceptable carriers known in the art such as vehicles (e.g., lactose, white sugar, mannitol, glucose, starches, calcium carbonate, crystalline cellulose, silicic acid, and the like), binders (e.g., water, ethanol, myranol, glucose solution, starch solution, gelatin solution, polyvinylpyrrolidone, and the like), disintegrators (e.g., dry starch, sodium, alginate, sodium hydrogen carbonate, calcium carbonate, polyoxyethylene sorbitan fatty acid esters, sodium laurylsulfate, stearic monoglyceride, starches, lactose, and the like), absorption promoters (e.g., quaternary ammonium base, sodium laurylsulfate, and the like), wetting agents (e.g. glycerin, starches, and the like), lubricants (e.g., stearates, polyethylene glycol, and the like), and flavoring agents (e.g., sweeteners). The tablets can be in the form of a conventional tablet, a molded tablet, a wafer and the like. Sublingual administration refers to the administration in the mouth (e.g., under the tongue, between the cheek and gum, between the tongue and roof of the mouth). The highly vascular mucosal lining in the mouth is a convenient location for the active ingredients to be administered into the body.

In other embodiments, the solid dosage form can be packaged as granules or a powder in a pharmaceutically acceptable carrier, where the granules or powder are removed from the packaging and sprinkled on food or mixed with a liquid, such as water or juice, or where the granules are inserted into capsules. In this embodiment, the active ingredients described herein can be mixed with flavoring or sweetening agents. The packaging material can be plastic, coated paper, or any material that prevents water or moisture from reaching the granules and/or powder.

Liquid dosage fauns for oral administration can include pharmaceutically acceptable gels, emulsions, solutions, sublingual solutions, suspensions, and syrups containing inert diluents commonly used in the art, such as water. Such compositions can also comprise adjuvants, such as wetting agents, emulsifying and suspending agents, and sweetening, flavoring, and perfuming agents. To make sublingual solutions, the active ingredients can be admixed with various carriers, excipients, pH adjusters, and the like (e.g., water, sugar, lactic acid, acetic acid, fructose, glucose, saccharin, polyethylene glycol, propylene glycol, alcohol, bentonite, tragacanth, gelatin, alginates, aspartame, sorbitol, methylparaben, propylparaben, sodium benzoate, artificial flavoring and coloring agents).

Hereinafter, the present invention will be illustrated by way of specific examples, although the invention should not be limited thereto.

### Example

A clinical trial in combination with a cinnamide compound of the present invention and a cholinesterase inhibitor on patients with Alzheimer's disease.

Male and female patients aging from 60 to 85 suffering from mild to moderate Alzheimer's disease who fulfill the NINCDS-ADRDA criteria will be recruited, and 50 to 100 patients will be registered as a cinnamide compound treatment group for the main test. Physicians judge the patients who will be applied for the main test based on the NINCDS-ADRDA criteria before administration. The physicians also will diagnose patients' clinical signs for dementia and mild cognitive impairments, such as disorientation, impaired memory, impaired judgment, impaired intellect, anxiety, depression, agitation and deterioration in activity in daily life and will judge based on all information available at the point of analysis such as report from family members or care attendants, and period of care. Alzheimer's disease subjects will be attended by care attendants who will make sure of compliance with dosing regimen, hospital visit and procedure for this test. Patients suffering from other disease with a medically severe symptom, patients with any contraindication or patients who regularly will use cinnamide compound will be eliminated. Patients receiving treatment with a cholinesterase inhibitor (e.g., donepezil hydrochloride) for Alzheimer's disease will not be eliminated if they will be administered with a stable dose in a range of about 3 to 20 mg/day for at least several weeks (preferably, 4 weeks).

To Alzheimer's disease patients who will fulfill the NINCDS-ADRDA criteria, cinnamide compound will be administered at an amount formulated in a carrier appropriate for the selected dosing regimen. The patients will start with cinnamide compound treatment protocol. An amount of cinnamide compound in a range of about 10 to 500 mg/day, for example, 100 mg/day will be prescribed in 1 to 3 doses. During the test period, administration will be repeated daily. Starting from day 0, a test for assessment scale will be conducted regularly, for example, once a month.

The cinnamide compound treatment group will be evaluated for one or several assessment items including, for example, a test for cognitive and memory function, A beta present in cerebrospinal cord fluid or plasma, A beta deposition in the brain, brain amyloid plaque, or a volume of a brain or hippocampus. In the cinnamide compound treatment group, progression of the disease will be inhibited or delayed or a symptom of the disease will be alleviated as compared to the untreated control group. Specifically, results will be obtained where the degree or period of patients' disorientation, impaired memory, impaired judgment and impaired intellect will be reduced, patients' anxiety, depression, agitation, frustration or fecklessness will be reduced, or patients start to feel stable, will become socially active, or will become to have improved language ability or comprehension.

Each of the patents, patent applications, and publications cited herein are incorporated by reference herein in their entirety.

It will be apparent to one skilled in the art that various modifications can be made to the invention without departing from the spirit or scope of the appended claims.

1. A pharmaceutical composition comprising:
(A) a compound of Formula (I) or a pharmaceutically acceptable salt thereof: 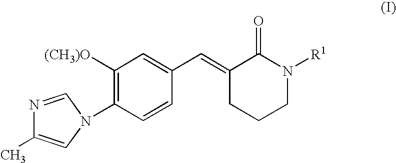
wherein R¹ is:
(1) —X₁—Ar₁, wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;
(2) an indenyl group optionally substituted with 1 to 3 halogen atoms;
(3) a tetrahydronaphthyl group optionally substituted with 1 to 3 halogen atoms; or
(4) a chromanyl group optionally substituted with 1 to 3 halogen atoms;
(B) a cholinesterase inhibitor; an AMPA receptor antagonist; an NMDA receptor antagonist; or a mixture or combination of two or more thereof; and
(C) one or more pharmaceutically acceptable carriers.

2. The pharmaceutical composition of claim 1, wherein R¹ is —X₁—Ar₁; wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

3. The pharmaceutical composition of claim 1, wherein R¹ is an indenyl group optionally substituted with 1 to 3 halogen atoms; a tetrahydronaphthyl group optionally substituted with 1 to 3 halogen atoms; or a chromanyl group optionally substituted with 1 to 3 halogen atoms.

4. The pharmaceutical composition of claim 1, wherein (A) is at least one compound selected from: (E)-1-(3,4-difluorobenzyl)-3-[3-methoxy-4-(4-methyl-1H-imidazol-1-yl)benzylidene]piperidin-2-one or a pharmaceutically acceptable salt thereat
(E)-1-indan-2-yl-3-[3-methoxy-4-(4-methyl-1H-imidazol-1-yl)benzylidene]piperidin-2-one or a pharmaceutically acceptable salt thereof;
(E)-1-[(4R)-chroman-4-yl]-3-[3-methoxy-4-(4-methyl-1H-imidazol-1-yl)benzylidene]piperidin-2-one or a pharmaceutically acceptable salt thereat (E)-1-[(4S)-chroman-4-yl]-3-[3-methoxy-4-(4-methyl-1H-imidazol-1-yl)benzylidene]piperidin-2-one or a pharmaceutically acceptable salt thereof;
(E)-1-[(1S)-1-(4-fluorophenyl)ethyl]-3-[3-methoxy-4-(4-methyl-1H-imidazol-1-yl)benzylidene]piperidin-2-one or a pharmaceutically acceptable salt thereat
(E)-1-(4-tert-butylbenzyl)-3-[3-methoxy-4-(4-methyl-1H-imidazol-1-yl)benzylidene]piperidin-2-one or a pharmaceutically acceptable salt thereof;
(E)-1-[(2S)-5-fluoroindan-2-yl]-3-[3-methoxy-4-(4-methyl-1H-imidazol-1-yl)benzylidene]piperidin-2-one or a pharmaceutically acceptable salt thereof; (E)-1-[(2R)-5-fluoroindan-2-yl]-3-[3-methoxy-4-(4-methyl-1H-imidazol-1-yl)benzylidene]piperidin-2-one or a pharmaceutically acceptable salt thereof;
(E)-1-[(4R)-7-fluorochroman-4-yl]-3-[3-methoxy-4-(4-methyl-1H-imidazol-1-yl)benzylidene]piperidin-2-one or a pharmaceutically acceptable salt thereof; or (E)-1-[(48)-7-fluorochroman-4-yl]-3-[3-methoxy-4-(4-methyl-1H-imidazol-1-yl)benzylidene]piperidin-2-one or a pharmaceutically acceptable salt thereof;
(E)-3-[3-methoxy-4-(4-methyl-1H-imidazol-1-yl)benzylidene]-1-[(2R)-1,2,3,4-tetrahydronaphthalen-2-yl]piperidin-2-one or a pharmaceutically acceptable salt thereof; (E)-3-[3-methoxy-4-(4-methyl-1H-imidazol-1-yl)benzylidene]-1-[(2S)-1,2,3,4-tetrahydronaphthalen-2-yl]piperidin-2-one or a pharmaceutically acceptable salt thereof; and
(E)-1-[(1R)-1-(2,4-difluorophenyl)ethyl]-3-[3-methoxy-4-(4-methyl-1H-imidazol-1-yl)benzylidene]piperidin-2-one or a pharmaceutically acceptable salt thereof; or (E)-1-[(1S)-1-(2,4-difluorophenyl)ethyl]-3-[3-methoxy-4-(4-methyl-1H-imidazol-1-yl)benzylidene]piperidin-2-one or a pharmaceutically acceptable salt thereof.

5. The pharmaceutical composition of claim 1, wherein (B) is one or more compounds selected from the group consisting of donepezil or a pharmaceutically acceptable salt thereof, huperzine A or a pharmaceutically acceptable salt thereof, tacrine or a pharmaceutically acceptable salt thereof, rivastigmine or a pharmaceutically acceptable salt thereof, galantamine or a pharmaceutically acceptable salt thereof, pramiracetam or a pharmaceutically acceptable salt thereof, aniracetam or a pharmaceutically acceptable salt thereof, nefiracetam or a pharmaceutically acceptable salt thereof; EGb 761 or a pharmaceutically acceptable salt thereof; rosiglitazone or a pharmaceutically acceptable salt thereof, rasagiline or a pharmaceutically acceptable salt thereof; levacecarnine or a pharmaceutically acceptable salt thereof, celecoxib or a pharmaceutically acceptable salt thereof, 3-(2-cyanophenyl)-5-(2-pyridyl)-1-phenyl-1,2-dihydropyridin-2-one, a pharmaceutically acceptable salt thereof or a hydrate thereof; talampanel or a pharmaceutically acceptable salt thereof; becampanel or a pharmaceutically acceptable salt thereof; memantine or a pharmaceutically acceptable salt thereof, neramexane or a pharmaceutically acceptable salt thereof; xaliproden or a pharmaceutically acceptable salt thereof; tarenflurbil or a pharmaceutically acceptable salt thereof; tramiprosate or a pharmaceutically acceptable salt thereof; and leuprorelin-D or a pharmaceutically acceptable salt thereof.

6. The pharmaceutical composition of claim 1, wherein (B) is one or more compounds selected from the group consisting of donepezil or a pharmaceutically acceptable salt thereof; 3-(2-cyanophenyl)-5-(2-pyridyl)-1-phenyl-1,2-dihydropyridin-2-one, a pharmaceutically acceptable salt thereof or a hydrate thereof and memantine or a pharmaceutically acceptable salt thereof.

7. The pharmaceutical composition of claim 1, wherein (B) is donepezil or a pharmaceutically acceptable salt thereof.

8. The pharmaceutical composition of claim 1, wherein the composition is used for: treating dementia or one or more mild cognitive impairments; the prophylaxis of dementia or one or more mild cognitive impairments; or delaying the onset of dementia or one or more mild cognitive impairments.

9. A combination comprising:
(A) a compound of Formula (I) or a pharmaceutically acceptable salt thereof: 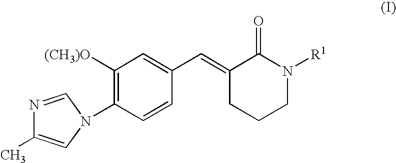
wherein R¹ is:
(1) —X₁—Ar₁, wherein Xₜ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;
(2) an indenyl group optionally substituted with 1 to 3 halogen atoms;
(3) a tetrahydronaphthyl group optionally substituted with 1 to 3 halogen atoms; or
(4) a chromanyl group optionally substituted with 1 to 3 halogen atoms; and
(B) a cholinesterase inhibitor; an AMPA receptor antagonist; an NMDA receptor antagonist; or a mixture or combination of two or more thereof.

10. The combination of claim 9, wherein R¹ is —X₁—Ar₁; wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

11. The combination of claim 9, wherein R¹ is an indenyl group optionally substituted with 1 to 3 halogen atoms; a tetrahydronaphthyl group optionally substituted with 1 to 3 halogen atoms; or a chromanyl group optionally substituted with 1 to 3 halogen atoms.

12. The combination of claim 9, wherein (A) is at least one compound selected from: (E)-1-(3,4-difluorobenzyl)-3-[3-methoxy-4-(4-methyl-1H-imidazol-1-yl)benzylidene]piperidin-2-one or a pharmaceutically acceptable salt thereof;
(E)-1-indan-2-yl-3-[3-methoxy-4-(4-methyl-1H-imidazol-1-yl)benzylidene]piperidin-2-one or a pharmaceutically acceptable salt thereof;
(E)-1-[(4R)-chroman-4-yl]-3-[3-methoxy-4-(4-methyl-1H-imidazol-1-yl)benzylidene]piperidin-2-one or a pharmaceutically acceptable salt thereof; (E)-1-[(4S)-chroman-4-yl]-3-[3-methoxy-4-(4-methyl-1H-imidazol-1-yl)benzylidene]piperidin-2-one or a pharmaceutically acceptable salt thereof;
(E)-1-[(1S)-1-(4-fluorophenyl)ethyl]-3-[3-methoxy-4-(4-methyl-1H-imidazol-1-yl)benzylidene]piperidin-2-one or a pharmaceutically acceptable salt thereof;
(E)-1-(4-tert-butylbenzyl)-3-[3-methoxy-4-(4-methyl-1H-imidazol-1-yl)benzylidene]piperidin-2-one or a pharmaceutically acceptable salt thereof;
(E)-1-[(2S)-5-fluoroindan-2-yl]-3-[3-methoxy-4-(4-methyl-1H-imidazol-1-yl)benzylidene]piperidin-2-one or a pharmaceutically acceptable salt thereof; (E)-1-[(2R)-5-fluoroindan-2-yl]-3-[3-methoxy-4-(4-methyl-1H-imidazol-1-yl)benzylidene]piperidin-2-one or a pharmaceutically acceptable salt thereof;
(E)-1-[(4R)-7-fluorochroman-4-yl]-3-[3-methoxy-4-(4-methyl-1H-imidazol-1-yl)benzylidene]piperidin-2-one or a pharmaceutically acceptable salt thereof; or (E)-1-[(4S)-7-fluorochroman-4-yl]-3-[3-methoxy-4-(4-methyl-1H-imidazol-1-yl)benzylidene]piperidin-2-one or a pharmaceutically acceptable salt thereof;
(E)-3-[3-methoxy-4-(4-methyl-1H-imidazol-1-yl)benzylidene]-1-[(2R)-1,2,3,4-tetrahydronaphthalen-2-yl]piperidin-2-one or a pharmaceutically acceptable salt thereof; (E)-3-[3-methoxy-4-(4-methyl-1H-imidazol-1-yl)benzylidene]-1-[(2S)-1,2,3,4-tetrahydronaphthalen-2-yl]piperidin-2-one or a pharmaceutically acceptable salt thereof; and
(E)-1-[(1R)-1-(2,4-difluorophenyl)ethyl]-3-[3-methoxy-4-(4-methyl-1H-imidazol-1-yl)benzylidene]piperidin-2-one or a pharmaceutically acceptable salt thereof; or (E)-1-[(1S)-1-(2,4-difluorophenyl)ethyl]-3-[3-methoxy-4-(4-methyl-1H-imidazol-1-yl)benzylidene]piperidin-2-one or a pharmaceutically acceptable salt thereof

13. The combination of claim 9, wherein (B) is one or more compounds selected from the group consisting of donepezil or a pharmaceutically acceptable salt thereof, huperzine A or a pharmaceutically acceptable salt thereof, tacrine or a pharmaceutically acceptable salt thereof, rivastigmine or a pharmaceutically acceptable salt thereof, galantamine or a pharmaceutically acceptable salt thereof, pramiracetam or a pharmaceutically acceptable salt thereof, aniracetam or a pharmaceutically acceptable salt thereof, nefiracetam or a pharmaceutically acceptable salt thereof, EGb 761 or a pharmaceutically acceptable salt thereof, rosiglitazone or a pharmaceutically acceptable salt thereof, rasagiline or a pharmaceutically acceptable salt thereof, levacecarnine or a pharmaceutically acceptable salt thereof, celecoxib or a pharmaceutically acceptable salt thereof, 3-(2-cyanophenyl)-5(2-pyridyl)-1-phenyl-1,2-dihydropyridin-2-one, a pharmaceutically acceptable salt thereof or a hydrate thereof, talampanel or a pharmaceutically acceptable salt thereof, becampanel or a pharmaceutically acceptable salt thereof; memantine or a pharmaceutically acceptable salt thereof, neramexane or a pharmaceutically acceptable salt thereof, xaliproden or a pharmaceutically acceptable salt thereof, tarenflurbil or a pharmaceutically acceptable salt thereof, tramiprosate or a pharmaceutically acceptable salt thereof, and leuprorelin-D or a pharmaceutically acceptable salt thereof.

14. The combination of claim 9, wherein (B) is one or more compounds selected from the group consisting of donepezil or a pharmaceutically acceptable salt thereof, 3-(2-cyanophenyl)-5-(2-pyridyl)-1-phenyl-1,2-dihydropyridin-2-one, a pharmaceutically acceptable salt thereof or a hydrate thereof and memantine or a pharmaceutically acceptable salt thereof.

15. The combination of claim 9, wherein (B) is donepezil or a pharmaceutically acceptable salt thereof.

16. The combination of claim 9, wherein (A) and (B) are administered separately to a patient or are administered to a patient in the form of a pharmaceutical composition.

17. The combination of claim 9, wherein the combination is used for treating dementia or one or more mild cognitive impairments; for the prophylaxis of dementia or one or more mild cognitive impairments; or delaying the onset of dementia or one or more mild cognitive impairments.

18. A method for producing a pharmaceutical composition comprising compounds (A) and (B) in the treatment of dementia or one or more mild cognitive impairments; for the prophylaxis of dementia or one or more mild cognitive impairments; or delaying the onset of dementia or one or more mild cognitive impairments, wherein (A) and (B) are:
(A) a compound of Formula (I) or a pharmaceutically acceptable salt thereof: 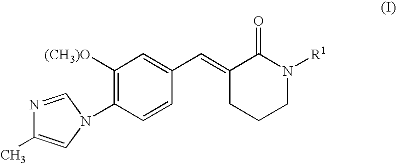
wherein R¹ is:
(1) —X₁—Ar₁, wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;
(2) an indenyl group optionally substituted with 1 to 3 halogen atoms;
(3) a tetrahydronaphthyl group optionally substituted with 1 to 3 halogen atoms; or
(4) a chromanyl group optionally substituted with 1 to 3 halogen atoms; and
(B) a cholinesterase inhibitor; an AMPA receptor antagonist; an NMDA receptor antagonist; or a mixture or combination of two or more thereof.

19. The method of claim 18, wherein R¹ is —X₁—Ar₁; wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;

20. The method of claim 18, wherein R¹ is an indenyl group optionally substituted with 1 to 3 halogen atoms; a tetrahydronaphthyl group optionally substituted with 1 to 3 halogen atoms; or a chromanyl group optionally substituted with 1 to 3 halogen atoms.

21. The method of claim 18, wherein (A) is at least one compound selected from: (E)-1-(3,4-difluorobenzyl)-3-[3-methoxy-4-(4-methyl-1H-imidazol-1-yl)benzylidene]piperidin-2-one or a pharmaceutically acceptable salt thereof;
(E)-1-indan-2-yl-3-[3-methoxy-4-(4-methyl-1H-imidazol-1-yl)benzylidene]piperidin-2-one or a pharmaceutically acceptable salt thereof;
(E)-1-[(4R)-chroman-4-yl]-3-[3-methoxy-4-(4-methyl-1H-imidazol-1-yl)benzylidene]piperidin-2-one or a pharmaceutically acceptable salt thereof; (E)-1-[(4S)-chroman-4-yl]-3-[3-methoxy-4-(4-methyl-1H-imidazol-1-yl)benzylidene]piperidin-2-one or a pharmaceutically acceptable salt thereof;
(E)-1-[(1S)-1-(4-fluorophenyl)ethyl]-3-[3-methoxy-4-(4-methyl-1H-imidazol-1-yl)benzylidene]piperidin-2-one or a pharmaceutically acceptable salt thereof;
(E)-1-(4-tert-butylbenzyl)-3-[3-methoxy-4-(4-methyl-1H-imidazol-1-yl)benzylidene]piperidin-2-one or a pharmaceutically acceptable salt thereof;
(E)-1-[(2S)-5-fluoroindan-2-yl]-3-[3-methoxy-4-(4-methyl-1H-imidazol-1-yl)benzylidene]piperidin-2-one or a pharmaceutically acceptable salt thereof; (E)-1-[(2R)-5-fluoroindan-2-yl]-3-[3-methoxy-4-(4-methyl-1H-imidazol-1-yl)benzylidene]piperidin-2-one or a pharmaceutically acceptable salt thereof;
(E)-1-[(4R)-7-fluoro chroman-4-yl]-3-[3-methoxy-4-(4-methyl-1H-imidazol-1-yl)benzylidene]piperidin-2-one or a pharmaceutically acceptable salt thereof; or (E)-1-[(4S)-7-fluorochroman-4-yl]-3-[3-methoxy-4-(4-methyl-1H-imidazol-1-yl)benzylidene]piperidin-2-one or a pharmaceutically acceptable salt thereof;
(E)-3-[3-methoxy-4-(4-methyl-1H-imidazol-1-yl)benzylidene]-1-[(2R)-1,2,3,4-tetrahydronaphthalen-2-yl]piperidin-2-one or a pharmaceutically acceptable salt thereof; (E)-3-[3-methoxy-4-(4-methyl-1H-imidazol-1-yl)benzylidene]-1-[(2S)-1,2,3,4-tetrahydronaphthalen-2-yl]piperidin-2-one or a pharmaceutically acceptable salt thereof; and
(E)-1-[(1R)-1-(2,4-difluorophenyl)ethyl]-3-[3-methoxy-4-(4-methyl-1H-imidazol-1-yl)benzylidene]piperidin-2-one or a pharmaceutically acceptable salt thereof; or (E)-1-[(1S)-1-(2,4-difluorophenyl)ethyl]-3-[3-methoxy-4-(4-methyl-1H-imidazol-1-yl)benzylidene]piperidin-2-one or a pharmaceutically acceptable salt thereof.

22. The method of claim 18, wherein (B) is one or more compounds selected from the group consisting of donepezil or a pharmaceutically acceptable salt thereof, huperzine A or a pharmaceutically acceptable salt thereof, tacrine or a pharmaceutically acceptable salt thereof, rivastigmine or a pharmaceutically acceptable salt thereof; galantamine or a pharmaceutically acceptable salt thereof; pramiracetam or a pharmaceutically acceptable salt thereof, aniracetam or a pharmaceutically acceptable salt thereof; nefiracetam or a pharmaceutically acceptable salt thereof; EGb 761 or a pharmaceutically acceptable salt thereof; rosiglitazone or a pharmaceutically acceptable salt thereof, rasagiline or a pharmaceutically acceptable salt thereof; levacecarnine or a pharmaceutically acceptable salt thereof; celecoxib or a pharmaceutically acceptable salt thereof, 3-(2-cyanophenyl)-5-(2-pyridyl)-1-phenyl-1,2-dihydropyridin-2-one, a pharmaceutically acceptable salt thereof or a hydrate thereof, talampanel or a pharmaceutically acceptable salt thereof, becampanel or a pharmaceutically acceptable salt thereof; memantine or a pharmaceutically acceptable salt thereof, neramexane or a pharmaceutically acceptable salt thereof, xaliproden or a pharmaceutically acceptable salt thereof, tarenflurbil or a pharmaceutically acceptable salt thereof; tramiprosate or a pharmaceutically acceptable salt thereof, and leuprorelin-D or a pharmaceutically acceptable salt thereof.

23. The method of claim 18, wherein (B) is one or more compounds selected from the group consisting of donepezil or a pharmaceutically acceptable salt thereof; 3-(2-cyanophenyl)-5-(2-pyridyl)-1-phenyl-1,2-dihydropyridin-2-one, a pharmaceutically acceptable salt thereof or a hydrate thereof and memantine or a pharmaceutically acceptable salt thereof.

24. The method of claim 18, wherein (B) is donepezil or a pharmaceutically acceptable salt thereof.

25. The method of claim 18, wherein (A) and (B) are to be administered separately to a patient or are administered to a patient in the form of a pharmaceutical composition.

26. A method for the treatment of dementia or one or more mild cognitive impairments; for the prophylaxis of dementia or one or more mild cognitive impairments; or delaying the onset of dementia or one or more mild cognitive impairments, by administering compounds (A) and (B), wherein (A) and (B) are:
(A) a compound of Formula (I) or a pharmaceutically acceptable salt thereof: 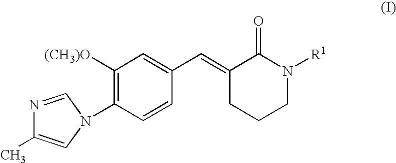
wherein R¹ is:
(1) —X₁—Ar₁, wherein X₁ is a C₁₋₆ alkylene group optionally substituted with a C₁₋₆ alkyl group; and Ar₁ is a phenyl group optionally substituted with 1 to 3 substituents selected from the group consisting of (i) a halogen atom and (ii) a C₁₋₆ alkyl group which may optionally be substituted with one to five C₁₋₆ alkyl groups;
(2) an indenyl group optionally substituted with 1 to 3 halogen atoms;
(3) a tetrahydronaphthyl group optionally substituted with 1 to 3 halogen atoms; or
(4) a chromanyl group optionally substituted with 1 to 3 halogen atoms; and
(B) a cholinesterase inhibitor; an AMPA receptor antagonist; an NMDA receptor antagonist; or a mixture or combination of two or more thereof.

27. A kit comprising the pharmaceutical composition of claim 1.

28. A method for treating dementia or one or more mild cognitive impairments; for the prophylaxis of dementia or one or more mild cognitive impairments; or
delaying the onset of dementia or one or more mild cognitive impairments comprising administering to a patient in need thereof a therapeutically effective amount of the pharmaceutical composition of claim 1.

29. A kit comprising the combination of claim 9.

30. A method for treating dementia or one or more mild cognitive impairments; for the prophylaxis of dementia or one or more mild cognitive impairments; or delaying the onset of dementia or one or more mild cognitive impairments comprising administering to a patient in need thereof a therapeutically effective amount of the combination of claim 9.
